(19) United States
(12) Patent Application Publication
(10) Pub. No.: US 2026/0234299 A1
(43) Pub. Date: Aug. 13, 2026

(54) PROCESSES FOR PRODUCING REACTION PRODUCTS INCLUDING QUATERNARY AMMONIUM SALTS

(71) Applicant: The Lubrizol Corporation, Wickliffe, OH (US)

(72) Inventors: Paul R. STEVENSON, Belper (GB); KAMALAKUMARI K. SALEM, Mentor, OH (US); DIXIT PARMAR, Long Eaton (GB); NICOLAS PROUST, Willowick, OH (US); PHILIP W. PIKE, Concord Township, OH (US); THOMAS D. MCALLISTER, Derbyshire (GB)

(73) Assignee: The Lubrizol Corporation, Wickliffe, OH (US)

(21) Appl. No.: 19/152,960

(22) PCT Filed: Feb. 2, 2024
(86) PCT No.: PCT/US2024/014123

§ 371 Date: Aug. 1, 2025

Related U.S. Application Data
(60) Provisional application No. 63/443,081, filed on Feb. 3, 2023.

(51) Int. Cl. C08F 8/44; C08F 8/32; C10L 1/232; C10L 10/18; C10M 149/02; C10N 40/25

(52) U.S. Cl. CPC C08F 8/44; C08F 8/32; C10L 1/232; C10L 10/18; C10M 149/02; C08F 2810/40; C10L 2230/086; C10M 2217/06; C10N 2040/25

(57) ABSTRACT

Disclosed is a process for producing a reaction product, the process including: reacting a hydrocarbyl polyisobutylene succinic anhydride with a nitrogen-containing compound to form a quaternizable compound; and reacting the quaternizable compound with a quaternizing agent to form the reaction product; wherein the reaction product includes a di-hydrocarbyl 5 succinic quaternary ammonium salt. Also disclosed are compositions including such reaction products and methods utilizing such compositions.

The disclosed technology relates to processes for producing reaction products which comprise quaternary ammonium salts. Also disclosed are compositions including such reaction products, methods utilizing such compositions, uses of such compositions in fuel compositions and/or lubricating compositions, and methods using and/or uses of such fuel compositions and/or lubricating compositions.

Deposit formation in diesel fuel injector nozzles is generally problematic, resulting in incomplete diesel combustion, and therefore power loss and/or misfiring. Traditionally, polyisobutylene succinimide detergents have been used to inhibit injector fouling, but these materials have shown poor efficacy in modern engines. A class of compounds based on quaternized polyisobutylene succinimides has been shown to provide improved detergency performance in both traditional and modern diesel engines.

Quaternary ammonium salts prepared from hydrocarbyl substituted acylating agents have been found to result in quaternary ammonium salts that, when blended into fuel, provide demulsification performance. It has now been found that utilizing a hydrocarbyl polyisobutylene succinic anhydride (as defined and described herein) to produce reaction products comprising quaternary ammonium salts, may provide alternative (and perhaps superior) quaternary ammonium salts for use as deposit control additives in fuels.

The subject matter disclosed herein provides a process for producing a reaction product, the process comprising: reacting a hydrocarbyl polyisobutylene succinic anhydride with a nitrogen-containing compound to form a quaternizable compound; and reacting the quaternizable compound with a quaternizing agent to form the reaction product; wherein the reaction product comprises a di-hydrocarbyl succinic quaternary ammonium salt.

The following embodiments of the present subject matter are contemplated:
1. A process for producing a reaction product, the process comprising: reacting a hydrocarbyl polyisobutylene succinic anhydride with a nitrogen-containing compound to form a quaternizable compound; and reacting the quaternizable compound with a quaternizing agent to form the reaction product; wherein the reaction product comprises a di-hydrocarbyl succinic quaternary ammonium salt.
2. The process of embodiment 1, wherein the nitrogen-containing compound has: (a) a nitrogen atom capable of reacting with the hydrocarbyl polyisobutylene succinic anhydride to form an imide and/or an amide; and (b) at least one quaternizable amino group.
3. The process of either embodiment 1 or embodiment 2, wherein the quaternizing agent is suitable for converting the quaternizable amino group to a quaternary nitrogen group.
4. The process of any one of embodiments 1 to 3, wherein the quaternizing agent comprises at least one of a dialkyl sulfate, an alkyl halide, an alkyl alkanoate, an aryl alkanoate, a hydrocarbyl substituted carbonate, or a hydrocarbyl epoxide.
5. The process of any one of embodiments 1 to 4, wherein the quaternizing agent comprises a hydrocarbyl epoxide in combination with an acid.
6. The process of any one of embodiments 1 to 5, wherein the di-hydrocarbyl quaternary ammonium salt comprises at least one compound of the following general formulas I or II:

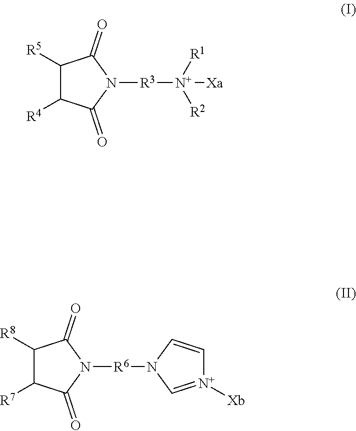

wherein, independently for each molecule of the compound: each R¹ is a hydrocarbyl group of from 1 to 10 carbon atoms; each R² is a hydrocarbyl group of from 1 to 10 carbon atoms; each R³ is a hydrocarbylene group of from 1 to 20 carbon atoms; each R⁴ is a hydrocarbyl group of from 20 to 180 carbon atoms; each R⁵ is a hydrocarbyl group of from 1 to 10 carbon atoms; each R⁶ is a hydrocarbylene group of from 1 to 20 carbon atoms; each R⁷ is a hydrocarbyl group of from 20 to 180 carbon atoms; each R⁸ is a hydrocarbyl group of from 1 to 10 carbon atoms; each Xa is a group derived from the quaternizing agent; and each Xb is a group derived from the quaternizing agent.
7. The process of any one of embodiments 1 to 6, wherein the hydrocarbyl polyisobutylene succinic anhydride comprises at least one compound of the following general formula III:

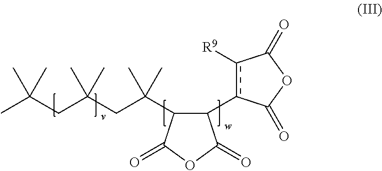

wherein, independently for each molecule: R⁹ is a hydrocarbyl group of from 1 to 10 carbon atoms or is not present; wherein when R⁹ is not present, the dashed double bond represents a double bond, and when R⁹ is present, the dashed double bond represents a single bond; v is an integer from 1 to 52; and w is 0, 1 or 2.
8. The process of embodiment 7, wherein R⁹ is present and the dashed double bond represents a single bond.
9. The process of embodiment 8, wherein R⁹ is a methyl group, an ethyl group, or an isobutylene group.
10. The process of either embodiment 8 or embodiment 9, wherein R⁹ is an isobutylene group.
11. A composition comprising the reaction product of the process of any one of embodiments 1 to 10.
12. A fuel composition comprising a fuel and the composition of embodiment 11.
13. A lubricating composition comprising an oil of lubricating viscosity and the composition of embodiment 11.
14. A method of improving water shedding performance of a fuel composition, the method comprising incorporating the composition of embodiment 11 into the fuel composition.
15. A method of reducing and/or preventing injector deposits in an engine, the method comprising supplying the fuel composition of embodiment 12 to a fuel injector of the engine and operating the engine.
16. A method of lubricating the crankcase of an engine, the method comprising supplying the lubricating composition of embodiment 13 to the engine and operating the engine.
17. The use of the fuel composition of embodiment 12 to reduce and/or prevent internal deposits in an engine operated with the fuel composition.
18. The use of the lubricating composition of embodiment 13 to lubricate the crankcase of an engine.
19. A composition comprising a di-hydrocarbyl quaternary ammonium salt according to at least one of the following general formulas I or II:

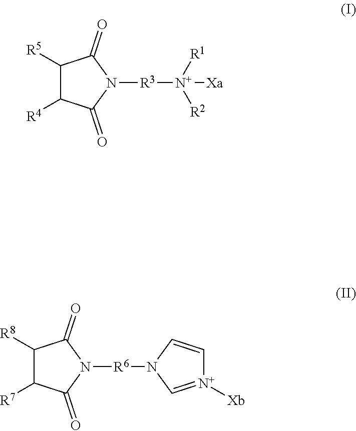

wherein, independently for each molecule of the compound: each R¹ is a hydrocarbyl group of from 1 to 10 carbon atoms; each R² is a hydrocarbyl group of from 1 to 10 carbon atoms; each R³ is a hydrocarbylene group of from 1 to 20 carbon atoms; each R⁴ is a hydrocarbyl group of from 20 to 180 carbon atoms; each R⁵ is a hydrocarbylene group of from 1 to 10 carbon atoms; each R⁶ is a hydrocarbylene group of from 1 to 20 carbon atoms; each R⁷ is a hydrocarbyl group of from 20 to 180 carbon atoms; each R⁸ is a hydrocarbylene group of from 1 to 10 carbon atoms; each Xa is a group derived from the quaternizing agent; and each Xb is a group derived from the quaternizing agent.
20. A fuel composition comprising a fuel and the composition of embodiment 19.
21. A lubricating composition comprising an oil of lubricating viscosity and the composition of embodiment 19.
22. A method of improving water shedding performance of a fuel composition, the method comprising incorporating the composition of embodiment 19 into the fuel composition.
23. A method of reducing and/or preventing injector deposits in an engine, the method comprising supplying the fuel composition of embodiment 20 to a fuel injector of the engine and operating the engine.
24. A method of lubricating the crankcase of an engine, the method comprising supplying the lubricating composition of embodiment 21 to the engine and operating the engine.
25. The use of the fuel composition of embodiment 20 to reduce and/or prevent internal deposits in an engine operated with the fuel composition.
26. The use of the lubricating composition of embodiment 21 to lubricate the crankcase of an engine.

Various features and embodiments of the present subject matter will be described below by way of non-limiting illustration.

The amount of each chemical component described herein is presented exclusive of any solvent or diluent oil, which may be customarily present in the commercial material, that is, on an active chemical basis, unless otherwise indicated. Unless otherwise indicated, each chemical or composition referred to herein should be interpreted as being a commercial grade material which may contain the isomers, by-products, derivatives, and other such materials which are normally understood to be present in the commercial grade.

As used herein, the term “hydrocarbyl” refers to a group having a carbon atom directly attached to the remainder of the molecule, where the group includes at least carbon and hydrogen atoms. If the hydrocarbyl group comprises more than one carbon atom, then those carbons need not necessarily be linked to each other. For example, at least two of the carbons may be linked via a suitable element or group. In various embodiments, the term “hydrocarbyl” refers to a group having a carbon atom directly attached to the remainder of the molecule, where the group consists of carbon, hydrogen, optionally one or more heteroatoms provided the heteroatoms do not alter the predominantly hydrocarbon nature of the substituent. The heteroatom may link to at least two of the carbons in the hydrocarbyl group, and optionally no more than two non-hydrocarbon substituents. Suitable heteroatoms will be apparent to those skilled in the art and include, for instance, sulfur, nitrogen, oxygen, phosphorus and silicon. Where the hydrocarbyl contains heteroatoms, optionally, no more than two heteroatoms will be present for every ten carbon atoms in the hydrocarbyl group. Suitable non-hydrocarbon substituents will also be apparent to those skilled in the art and include, for instance, halo, hydroxy, alkoxy, mercapto, alkylmercapto, nitro, nitroso, and sulfoxy.

Examples of hydrocarbyls within the context of the present technology therefore include: (i) hydrocarbon groups selected from aliphatic (e.g. alkyl or alkenyl), alicyclic (e.g. cycloalkyl, cycloalkenyl, cycloalkadienyl), and aromatic groups; (ii) substituted hydrocarbon groups, selected from hydrocarbon groups defined in (i) substituted with no more than two non-hydrocarbon substituents and/or one or more hydrocarbon substituents, the non-hydrocarbon substituents being selected from the group consisting of halo, hydroxy, alkoxy, mercapto, alkylmercapto, nitro, nitroso, and sulfoxy; and/or (iii) hetero-containing hydrocarbon groups, selected from hydrocarbon groups defined in (i) containing one or more heteroatom in the ring or chain, provided that the group has no more than two heteroatoms present for every ten carbon atoms in the group, the heteroatoms being selected from sulfur, nitrogen, oxygen, phosphorus and silicon. The hetero-containing hydrocarbon groups may be substituted with no more than two non-hydrocarbon substituents and/or one or more hydrocarbon substituents. In certain embodiments, the term “hydrocarbyl” refers to a group having a carbon atom directly attached to the remainder of the molecule, where the group consists of carbon and hydrogen atoms.

It is known that some of the materials described herein may interact in the final formulation, so that the components of the final formulation may be different from those that are initially added. For instance, metal ions (of, e.g., a detergent) may migrate to other acidic or anionic sites of other molecules. The products formed thereby, including the products formed upon employing the composition of the present subject matter in its intended use, may not be susceptible of easy description. Nevertheless, all such modifications and reaction products are included within the scope of the present subject matter; the present subject matter encompasses the composition prepared by admixing the components described herein.

As used herein, the indefinite article “a”/“an” is intended to mean one or more than one. As used herein, the phrase “at least one” means one or more than one of the following terms. Thus, “a”/“an” and “at least one” may be used interchangeably. For example “at least one of A, B or C” means that just one of A, B or C may be included, and any mixture of two or more of A, B and C may be included, in alternative embodiments.

As used herein, the term “substantially free of” means that a component does not include any intentional addition of the material which the component is “substantially free of”. For example, the component may include a material which the component is “substantially free of” at no more than impurity levels, which may be the result of incomplete chemical reactions and/or unintended/undesired (but perhaps unavoidable) reaction products.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

### Reaction Product Comprising a Di-Hydrocarbyl Succinic Quaternary Ammonium Salt

Provided is a process for producing a reaction product, the process comprising: reacting a hydrocarbyl polyisobutylene succinic anhydride with a nitrogen-containing compound to form a quaternizable compound; and reacting the quaternizable compound with a quaternizing agent to form the reaction product; wherein the reaction product comprises a di-hydrocarbyl succinic quaternary ammonium salt. Production of reaction products including quaternary ammonium salts generally results in a mixture of compounds including a quaternary ammonium salt, and this mixture may be difficult to define apart from the process steps employed to produce the reaction products. As used herein, “di-hydrocarbyl succinic quaternary ammonium salt” refers to a molecule in which the hydrocarbyl groups are present on the succinic portion of the molecule.

In certain embodiments, the quaternary ammonium salt comprises an imide quaternary ammonium salt and/or an amide quaternary ammonium salt. In certain embodiments, the quaternary ammonium salt comprises at least 50 (such as 55, 60, 65, 70, 75, 80, 85, 90, 95, 96, 97, 98, or 99) percent by weight of an imide quaternary ammonium salt. In certain embodiments, the quaternary ammonium salt consists essentially of an imide quaternary ammonium salt. In certain embodiments, the quaternary ammonium salt consists of an imide quaternary ammonium salt. In certain embodiments, the quaternary ammonium salt is substantially free of an amide quaternary ammonium salt. In certain embodiments, the reaction product is substantially free of an amide quaternary ammonium salt.

In certain embodiments, the di-hydrocarbyl quaternary ammonium salt comprises at least one compound of the following general formulas I or II:

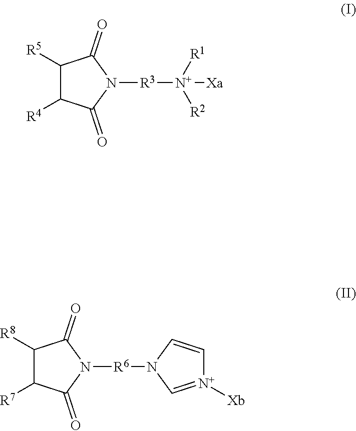

wherein, independently for each molecule of the compound: each R¹ is a hydrocarbyl group of from 1 to 10 carbon atoms; each R² is a hydrocarbyl group of from 1 to 10 carbon atoms; each R³ is a hydrocarbylene group of from 1 to 20 carbon atoms; each R⁴ is a hydrocarbyl group of from 20 to 180 carbon atoms; each R⁵ is a hydrocarbyl group of from 1 to 10 carbon atoms; each R⁶ is a hydrocarbylene group of from 1 to 20 carbon atoms; each R⁷ is a hydrocarbyl group of from 20 to 180 carbon atoms; each R⁸ is a hydrocarbyl group of from 1 to 10 carbon atoms; each Xa is a group derived from the quaternizing agent; and each Xb is a group derived from the quaternizing agent. In this context, the term “hydrocarbylene” refers to a group which is attached at two points to other carbon-containing groups.

Embodiments of the present technology described herein may provide the use of the reaction product comprising a di-hydrocarbyl succinic quaternary ammonium salt for at least one of antiwear performance, friction modification (such as for enhancing fuel economy), detergent performance (such as deposit control and/or varnish control), or dispersancy (such as soot control, sludge control, and/or corrosion control).

### Hydrocarbyl Polyisobutylene Succinic Anhydride

Acylated polyisobutylenes, such as polyisobutylene succinic anhydrides/acids (“PIBSA”), are used as chemical intermediates in producing a vast number of chemical compositions, such as the reaction products comprising quaternary ammonium salts described herein. As used herein, the terms “polyisobutylene succinic anhydride”, “polyisobutylene succinic acid”, “polyisobutylene succinic anhydride/acid”, and/or “PIBSA” mean any molecule (or composition comprising such molecules) which includes a polyisobutylene portion bonded (directly or indirectly) to a succinic anhydride/acid unit.

The hydrocarbyl polyisobutylene succinic anhydride is a PIBSA with a hydrocarbyl group pendant from the succinic portion of the PIBSA molecule, and may be made according to any known process. One process which may be used to make the hydrocarbyl polyisobutylene succinic anhydride is radical functionalization, as described in more detail below. In an alternative illustrative process, polyisobutylene may be reacted with an anhydride under nitrogen at a temperature of at least 210° C. and pressure of at least 1 bar for at least 6 hours to create a hydrocarbyl polyisobutylene succinic anhydride.

The hydrocarbyl polyisobutylene succinic anhydride may comprise at least one of a radically-functionalized PIBSA product (as described in more detail below) or the product of a reaction of polyisobutylene via an “ene” reaction with (i) citraconic acid or anhydride, (ii) 3-ethylfurane-2,5-dione acid, or (iii) [z]-2-ethyl-4-oxopent-2-enoic acid.

The hydrocarbyl polyisobutylene succinic anhydride may comprise at least one compound of the following general formula III:

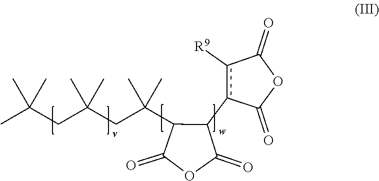

wherein, independently for each molecule: R⁹ is a hydrocarbyl group of from 1 to 10 carbon atoms or is not present; wherein when R⁹ is not present, the dashed double bond represents a double bond, and when R⁹ is present, the dashed double bond represents a single bond; v is an integer from 1 to 52; and w is 0, 1 or 2. In certain embodiments, R⁹ is present and the dashed double bond represents a single bond. In certain embodiments, R⁹ is a methyl group, an ethyl group, or an isobutylene group. In certain embodiments, R⁹ is an isobutylene group.

In certain embodiments, v is an integer from 1 to 53, from 1 to 52, from 1 to 51, from 1 to 50, from 1 to 49, from 1 to 48, from 1 to 46, from 1 to 44, from 1 to 42, from 1 to 40, from 1 to 38, from 1 to 36, from 1 to 34, from 1 to 32, from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 7, from 1 to 6, from 1 to 5, from 1 to 4, from 1 to 3, from 1 to 2, from 2 to 54, from 2 to 53, from 2 to 52, from 2 to 51, from 2 to 50, from 2 to 49, from 2 to 48, from 2 to 46, from 2 to 44, from 2 to 42, from 2 to 40, from 2 to 38, from 2 to 36, from 2 to 34, from 2 to 32, from 2 to 30, from 2 to 28, from 2 to 26, from 2 to 24, from 2 to 22, from 2 to 20, from 2 to 18, from 2 to 16, from 2 to 14, from 2 to 12, from 2 to 10, from 2 to 8, from 2 to 7, from 2 to 6, from 2 to 5, from 2 to 4, from 2 to 3, from 3 to 54, from 3 to 53 from 3 to 52, from 3 to 51, from 3 to 50, from 3 to 49, from 3 to 48, from 3 to 46, from 3 to 44, from 3 to 42, from 3 to 40, from 3 to 38, from 3 to 36, from 3 to 34, from 3 to 32, from 3 to 30, from 3 to 28, from 3 to 26, from 3 to 24, from 3 to 22, from 3 to 20, from 3 to 18, from 3 to 16, from 3 to 14, from 3 to 12, from 3 to 10, from 3 to 8, from 3 to 7, from 3 to 6, from 3 to 5, from 3 to 4, from 4 to 54, from 4 to 53, from 4 to 52, from 4 to 51, from 4 to 50, from 4 to 49, from 4 to 48, from 4 to 46, from 4 to 44, from 4 to 42, from 4 to 40, from 4 to 38, from 4 to 36, from 4 to 34, from 4 to 32, from 4 to 30, from 4 to 28, from 4 to 26, from 4 to 24, from 4 to 22, from 4 to 20, from 4 to 18, from 4 to 16, from 4 to 14, from 4 to 12, from 4 to 10, from 4 to 8, from 4 to 7, from 4 to 6, from 4 to 5, from 5 to 54, from 5 to 53, from 5 to 52, from 5 to 51, from 5 to 50, from 5 to 49, from 5 to 48, from 5 to 46, from 5 to 44, from 5 to 42, from 5 to 40, from 5 to 38, from 5 to 36, from 5 to 34, from 5 to 32, from 5 to 30, from 5 to 28, from 5 to 26, from 5 to 24, from 5 to 22, from 5 to 20, from 5 to 18, from 5 to 16, from 5 to 14, from 5 to 12, from 5 to 10, from 5 to 8, from 5 to 7, from 5 to 6, from 6 to 54, from 6 to 53, from 6 to 52, from 6 to 51, from 6 to 50, from 6 to 49, from 6 to 48, from 6 to 46, from 6 to 44, from 6 to 42, from 6 to 40, from 6 to 38, from 6 to 36, from 6 to 34, from 6 to 32, from 6 to 30, from 6 to 28, from 6 to 26, from 6 to 24, from 6 to 22, from 6 to 20, from 6 to 18, from 6 to 16, from 6 to 14, from 6 to 12, from 6 to 10, from 6 to 8, from 6 to 7, from 7 to 54, from 7 to 53, from 7 to 52, from 7 to 51, from 7 to 50, from 7 to 49, from 7 to 48, from 7 to 46, from 7 to 44, from 7 to 42, from 7 to 40, from 7 to 38, from 7 to 36, from 7 to 34, from 7 to 32, from 7 to 30, from 7 to 28, from 7 to 26, from 7 to 24, from 7 to 22, from 7 to 20, from 7 to 18, from 7 to 16, from 7 to 14, from 7 to 12, from 7 to 10, from 7 to 8, from 8 to 54, from 8 to 53, from 8 to 52, from 8 to 51, from 8 to 50, from 8 to 49, from 8 to 48, from 8 to 46, from 8 to 44, from 8 to 42, from 8 to 40, from 8 to 38, from 8 to 36, from 8 to 34, from 8 to 32, from 8 to 30, from 8 to 28, from 8 to 26, from 8 to 24, from 8 to 22, from 8 to 20, from 8 to 18, from 8 to 16, from 8 to 14, from 8 to 12, from 8 to 10, from 10 to 54, from 10 to 53, from 10 to 52, from 10 to 51, from 10 to 50, from 10 to 49, from 10 to 48, from 10 to 46, from 10 to 44, from 10 to 42, from 10 to 40, from 10 to 38, from 10 to 36, from 10 to 34, from 10 to 32, from 10 to 30, from 10 to 28, from 10 to 26, from 10 to 24, from 10 to 22, from 10 to 20, from 10 to 18, from 10 to 16, from 10 to 14, from 10 to 12, from 12 to 54, from 12 to 53, from 12 to 52, from 12 to 51, from 12 to 50, from 12 to 49, from 12 to 48, from 12 to 46, from 12 to 44, from 12 to 42, from 12 to 40, from 12 to 38, from 12 to 36, from 12 to 34, from 12 to 32, from 12 to 30, from 12 to 28, from 12 to 26, from 12 to 24, from 12 to 22, from 12 to 20, from 12 to 18, from 12 to 16, from 12 to 14, from 14 to 54, from 14 to 53, from 14 to 52, from 14 to 51, from 14 to 50, from 14 to 49, from 14 to 48, from 14 to 46, from 14 to 44, from 14 to 42, from 14 to 40, from 14 to 38, from 14 to 36, from 14 to 34, from 14 to 32, from 14 to 30, from 14 to 28, from 14 to 26, from 14 to 24, from 14 to 22, from 14 to 20, from 14 to 18, from 14 to 16, from 16 to 54, from 16 to 53, from 16 to 52, from 16 to 51, from 16 to 50, from 16 to 49, from 16 to 48, from 16 to 46, from 16 to 44, from 16 to 42, from 16 to 40, from 16 to 38, from 16 to 36, from 16 to 34, from 16 to 32, from 16 to 30, from 16 to 28, from 16 to 26, from 16 to 24, from 16 to 22, from 16 to 20, from 16 to 18, from 18 to 54, from 18 to 53, from 18 to 52, from 18 to 51, from 18 to 50, from 18 to 49, from 18 to 48, from 18 to 46, from 18 to 44, from 18 to 42, from 18 to 40, from 18 to 38, from 18 to 36, from 18 to 34, from 18 to 32, from 18 to 30, from 18 to 28, from 18 to 26, from 18 to 24, from 18 to 22, from 18 to 20, from 20 to 54, from 20 to 53, from 20 to 52, from 20 to 51, from 20 to 50, from 20 to 49, from 20 to 48, from 20 to 46, from 20 to 44, from 20 to 42, from 20 to 40, from 20 to 38, from 20 to 36, from 20 to 34, from 20 to 32, from 20 to 30, from 20 to 28, from 20 to 26, from 20 to 24, from 20 to 22, from 22 to 54, from 22 to 53, from 22 to 52, from 22 to 51, from 22 to 50, from 22 to 49, from 22 to 48, from 22 to 46, from 22 to 44, from 22 to 42, from 22 to 40, from 22 to 38, from 22 to 36, from 22 to 34, from 22 to 32, from 22 to 30, from 22 to 28, from 22 to 26, from 22 to 24, from 24 to 54, from 24 to 53, from 24 to 52, from 24 to 51, from 24 to 50, from 24 to 49, from 24 to 48, from 24 to 46, from 24 to 44, from 24 to 42, from 24 to 40, from 24 to 38, from 24 to 36, from 24 to 34, from 24 to 32, from 24 to 30, from 24 to 28, from 24 to 26, from 26 to 54, from 26 to 53, from 26 to 52, from 26 to 51, from 26 to 50, from 26 to 49, from 26 to 48, from 26 to 46, from 26 to 44, from 26 to 42, from 26 to 40, from 26 to 38, from 26 to 36, from 26 to 34, from 26 to 32, from 26 to 30, from 26 to 28, from 28 to 54, from 28 to 53, from 28 to 52, from 28 to 51, from 28 to 50, from 28 to 49, from 28 to 48, from 28 to 46, from 28 to 44, from 28 to 42, from 28 to 40, from 28 to 38, from 28 to 36, from 28 to 34, from 28 to 32, from 28 to 30, from 30 to 54, from 30 to 53, from 30 to 52, from 30 to 51, from 30 to 50, from 30 to 49, from 30 to 48, from 30 to 46, from 30 to 44, from 30 to 42, from 30 to 40, from 30 to 38, from 30 to 36, from 30 to 34, from 30 to 32, from 32 to 54, from 32 to 53, from 32 to 52, from 32 to 51, from 32 to 50, from 32 to 49, from 32 to 48, from 32 to 46, from 32 to 44, from 32 to 42, from 32 to 40, from 32 to 38, from 32 to 36, from 32 to 34, from 34 to 54, from 34 to 53, from 34 to 52, from 34 to 51, from 34 to 50, from 34 to 49, from 34 to 48, from 34 to 46, from 34 to 44, from 34 to 42, from 34 to 40, from 34 to 38, from 34 to 36, from 36 to 54, from 36 to 53, from 36 to 52, from 36 to 51, from 36 to 50, from 36 to 49, from 36 to 48, from 36 to 46, from 36 to 44, from 36 to 42, from 36 to 40, from 36 to 38, from 38 to 54, from 38 to 53, from 38 to 52, from 38 to 51, from 38 to 50, from 38 to 49, from 38 to 48, from 38 to 46, from 38 to 44, from 38 to 42, from 38 to 40, from 40 to 54, from 40 to 53, from 40 to 52, from 40 to 51, from 40 to 50, from 40 to 49, from 40 to 48, from 40 to 46, from 40 to 44, from 40 to 42, from 42 to 54, from 42 to 53, from 42 to 52, from 42 to 51, from 42 to 50, from 42 to 49, from 42 to 48, from 42 to 46, from 42 to 44, from 44 to 54, from 44 to 53, from 44 to 52, from 44 to 51, from 44 to 50, from 44 to 49, from 44 to 48, from 44 to 46, from 46 to 54, from 46 to 53, from 46 to 52, from 46 to 51, from 46 to 50, from 46 to 49, from 46 to 48, from 48 to 54, from 48 to 53, from 48 to 52, from 48 to 51, from 48 to 50, from 48 to 49, from 49 to 54, from 49 to 53, from 49 to 52, from 49 to 51, from 49 to 50, from 50 to 54, from 50 to 53, from 50 to 52, from 50 to 51, from 51 to 54, from 51 to 53, from 51 to 52, from 51 to 51, from 52 to 54, from 52 to 53, or from 53 to 54. In certain embodiments, v is 1, 2, 3, 4, 5, 6, 7, 8, 10, 12, 14, 16, 18, 20, 22, 24, 26, 28, 30, 32, 34, 36, 38, 40, 42, 44, 46, 48, 49, 50, 51, 52, 53, or 54.

In certain embodiments, the radically-functionalized PIBSA product may be a product of a process comprising: reacting polyisobutylene with a radical initiator and an ethylenically unsaturated acylating agent at a reaction temperature of from 150° C. to 225° C. to produce the product; wherein the polyisobutylene is brought to the reaction temperature prior to addition of the radical initiator; and wherein the ethylenically unsaturated acylating agent is added at any time prior to or during addition of the radical initiator. As such, the term “radically-functionalized PIBSA product”, as used herein, is intended to refer to the product of this reaction, optionally including the embodiments described below. The radically-functionalized PIBSA product therefore may include other products of such reactions in addition to the intended and/or desired PIBSA product, as would be understood by a person of ordinary skill in the art.

In certain embodiments, the ethylenically unsaturated acylating agent may be combined with the polyisobutylene at the beginning of the process. In certain embodiments, the ethylenically unsaturated acylating agent may be combined with the radical initiator for addition to the polyisobutylene after the polyisobutylene is brought to the reaction temperature. In certain embodiments, the process for producing the radically-functionalized PIBSA product may comprise reacting polyisobutylene (such as high-vinylidene polyisobutylene) with a radical initiator and an ethylenically unsaturated acylating agent at a reaction temperature of from 150° C. to 225° C. to produce the radically-functionalized PIBSA product, wherein the polyisobutylene is brought to the reaction temperature prior to addition of the radical initiator and the ethylenically unsaturated acylating agent.

As used herein, the term “high-vinylidene polyisobutylene” means a compound including at least 55 (such as at 60, at least 65, at least 70, at least 75, or at least 80) weight percent alpha-methyl-vinylidene polyisobutylene. In certain embodiments, the high-vinylidene polyisobutylene may comprise from 55 to 80 (such as from 55 to 75, from 55 to 70, from 55 to 65, from 55 to 60, from 60 to 80, from 60 to 75, from 60 to 70, from 60 to 65, from 65 to 80, from 65 to 75, from 65 to 70, from 70 to 80, from 70 to 75, or from 75 to 80) weight percent alpha-methyl-vinylidene polyisobutylene. High vinylidene polyisobutylenes are well known, are known to be highly reactive relative to other polyisobutylenes, and are useful as chemical intermediates in numerous chemical synthesis processes.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a temperature of from 150° C. to 225° C. to produce the radically-functionalized PIBSA product.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a reaction temperature of from 150° C. to 225° C. to produce the radically-functionalized PIBSA product, wherein each of the two or more additions comprises at least 5 weight percent of the total amount of the radical initiator and/or at least 5 weight percent of the total amount of the ethylenically unsaturated acylating agent to be reacted with the polyisobutylene.

In certain embodiments, the process for producing the radically-functionalized PIBSA product may comprise reacting polyisobutylene (such as high-vinylidene polyisobutylene) with a radical initiator and an ethylenically unsaturated acylating agent in two or more additions at a reaction temperature of from 150° C. to 225° C. to produce the radically-functionalized PIBSA product, wherein each of the two or more additions are separated in time by at least twice the half-life of the radical initiator.

In certain embodiments, the process for producing the radically-functionalized PIBSA product may comprise: reacting polyisobutylene (such as high-vinylidene polyisobutylene) with a radical initiator and an ethylenically unsaturated acylating agent in two or more additions at a reaction temperature of from 150° C. to 225° C. to produce the radically-functionalized PIBSA product; wherein each of the two or more additions comprises at least 5 weight percent of the total amount of the radical initiator and/or at least 5 weight percent of the total amount of the ethylenically unsaturated acylating agent to be reacted with the polyisobutylene; and wherein each of the two or more additions are separated in time by at least twice the half-life of the radical initiator.

In certain embodiments, the process for producing the radically-functionalized PIBSA product may comprise: reacting polyisobutylene (such as high-vinylidene polyisobutylene) with a radical initiator and an ethylenically unsaturated acylating agent at a temperature of from 150° C. to 225° C. to produce the radically-functionalized PIBSA product, wherein the polyisobutylene is brought to the reaction temperature in a continuous reactor, prior to commencing continuous addition of the radical initiator to the polyisobutylene in the continuous reactor; and wherein the ethylenically unsaturated acylating agent is added to the continuous reactor: (i) as a mixture with the polyisobutylene; (ii) as a mixture with the radical initiator; and/or (iii) continuously and separately from either the polyisobutylene or the radical initiator.

As compared with known processes for producing PIBSA, the processes for producing the radically-functionalized PIBSA product described above generally provide lower color products (e.g., free of dark specks/resins without filtration), ability to access a degree of functionality greater than 2, decreased energy consumption, reduced equipment maintenance, and/or decreased reaction times, as will become apparent in the detailed description and examples below. These and/or other features of the radically-functionalized PIBSA product are believed to contribute to the increased benefits of the reaction product comprising quaternary ammonium salts described herein.

In certain embodiments, the reaction temperature utilized in the processes described herein to produce the radically-functionalized PIBSA product may be from 155° C. to 225° C., from 160° C. to 225° C., from 165° C. to 225° C., from 166° C. to 225° C., from 167° C. to 225° C., from 168° C. to 225° C., from 169° C. to 225° C., from 170° C. to 225° C., from 171° C. to 225° C., from 172° C. to 225° C., from 173° C. to 225° C., from 174° C. to 225° C., from 175° C. to 225° C., from 150° C. to 220° C., from 155° C. to 220° C., from 160° C. to 220° C., from 165° C. to 220° C., from 166° C. to 220° C., from 167° C. to 220° C., from 168° C. to 220° C., from 169° C. to 220° C., from 170° C. to 220° C., from 171° C. to 220° C., from 172° C. to 220° C., from 173° C. to 220° C., from 174° C. to 220° C., from 175° C. to 220° C., from 150° C. to 215° C., from 155° C. to 215° C., from 160° C. to 215° C., from 165° C. to 215° C., from 166° C. to 215° C., from 167° C. to 215° C., from 168° C. to 215° C., from 169° C. to 215° C., from 170° C. to 215° C., from 171° C. to 215° C., from 172° C. to 215° C., from 173° C. to 215° C., from 174° C. to 215° C., from 175° C. to 215° C., from 150° C. to 210° C., from 155° C. to 210° C., from 160° C. to 210° C., from 165° C. to 210° C., from 166° C. to 210° C., from 167° C. to 210° C., from 168° C. to 210° C., from 169° C. to 210° C., from 170° C. to 210° C., from 171° C. to 210° C., from 172° C. to 210° C., from 173° C. to 210° C., from 174° C. to 210° C., from 175° C. to 210° C., from 150° C. to 205° C., from 155° C. to 205° C., from 160° C. to 205° C., from 165° C. to 205° C., from 166° C. to 205° C., from 167° C. to 205° C., from 168° C. to 205° C., from 169° C. to 205° C., from 170° C. to 205° C., from 171° C. to 205° C., from 172° C. to 205° C., from 173° C. to 205° C., from 174° C. to 205° C., from 175° C. to 205° C., from 150° C. to 200° C., from 155° C. to 200° C., from 160° C. to 200° C., from 165° C. to 200° C., from 166° C. to 200° C., from 167° C. to 200° C., from 168° C. to 200° C., from 169° C. to 200° C., from 170° C. to 200° C., from 171° C. to 200° C., from 172° C. to 200° C., from 173° C. to 200° C., from 174° C. to 200° C., from 175° C. to 200° C., from 150° C. to 195° C., from 155° C. to 195° C., from 160° C. to 195° C., from 165° C. to 195° C., from 166° C. to 195° C., from 167° C. to 195° C., from 168° C. to 195° C., from 169° C. to 195° C., from 170° C. to 195° C., from 171° C. to 195° C., from 172° C. to 195° C., from 173° C. to 195° C., from 174° C. to 195° C., from 175° C. to 195° C., from 150° C. to 190° C., from 155° C. to 190° C., from 160° C. to 190° C., from 165° C. to 190° C., from 166° C. to 190° C., from 167° C. to 190° C., from 168° C. to 190° C., from 169° C. to 190° C., from 170° C. to 190° C., from 171° C. to 190° C., from 172° C. to 190° C., from 173° C. to 190° C., from 174° C. to 190° C., from 175° C. to 190° C., from 150° C. to 185° C., from 155° C. to 185° C., from 160° C. to 185° C., from 165° C. to 185° C., from 166° C. to 185° C., from 167° C. to 185° C., from 168° C. to 185° C., from 169° C. to 185° C., from 170° C. to 185° C., from 171° C. to 185° C., from 172° C. to 185° C., from 173° C. to 185° C., from 174° C. to 185° C., from 175° C. to 185° C., from 150° C. to 180° C., from 155° C. to 180° C., from 160° C. to 180° C., from 165° C. to 180° C., from 166° C. to 180° C., from 167° C. to 180° C., from 168° C. to 180° C., from 169° C. to 180° C., from 170° C. to 180° C., from 171° C. to 180° C., from 172° C. to 180° C., from 173° C. to 180° C., from 174° C. to 180° C., from 175° C. to 180° C., from 150° C. to 175° C., from 155° C. to 175° C., from 160° C. to 175° C., from 165° C. to 175° C., from 166° C. to 175° C., from 167° C. to 175° C., from 168° C. to 175° C., from 169° C. to 175° C., from 170° C. to 175° C., from 171° C. to 175° C., from 172° C. to 175° C., from 173° C. to 175° C., from 174° C. to 175° C., from 150° C. to 174° C., from 155° C. to 174° C., from 160° C. to 174° C., from 165° C. to 174° C., from 166° C. to 174° C., from 167° C. to 174° C., from 168° C. to 174° C., from 169° C. to 174° C., from 170° C. to 174° C., from 171° C. to 174° C., from 172° C. to 174° C., from 173° C. to 174° C., from 150° C. to 173° C., from 155° C. to 173° C., from 160° C. to 173° C., from 165° C. to 173° C., from 166° C. to 173° C., from 167° C. to 173° C., from 168° C. to 173° C., from 169° C. to 173° C., from 170° C. to 173° C., from 171° C. to 173° C., from 172° C. to 173° C., from 150° C. to 172° C., from 155° C. to 172° C., from 160° C. to 172° C., from 165° C. to 172° C., from 166° C. to 172° C., from 167° C. to 172° C., from 168° C. to 172° C., from 169° C. to 172° C., from 170° C. to 172° C., from 171° C. to 172° C., from 150° C. to 171° C., from 155° C. to 171° C., from 160° C. to 171° C., from 165° C. to 171° C., from 166° C. to 171° C., from 167° C. to 171° C., from 168° C. to 171° C., from 169° C. to 171° C., from 170° C. to 171° C., from 150° C. to 170° C., from 155° C. to 170° C., from 160° C. to 170° C., from 165° C. to 170° C., from 166° C. to 170° C., from 167° C. to 170° C., from 168° C. to 170° C., or from 169° C. to 170° C.

In certain embodiments, the total amount of radical initiator added during the processes to produce the radically-functionalized PIBSA product described herein may be from 0.01 to 1 (such as from 0.01 to 0.9, from 0.01 to 0.8, from 0.01 to 0.7, from 0.01 to 0.6, from 0.01 to 0.5, from 0.01 to 0.45, from 0.01 to 0.4, from 0.01 to 0.35, from 0.01 to 0.3, from 0.01 to 0.25, from 0.01 to 0.2, from 0.01 to 0.19, from 0.01 to 0.18, from 0.01 to 0.17, from 0.01 to 0.16, from 0.01 to 0.15, from 0.01 to 0.14, from 0.01 to 0.13, from 0.01 to 0.12, from 0.01 to 0.11, from 0.01 to 0.1, from 0.02 to 0.9, from 0.02 to 0.8, from 0.02 to 0.7, from 0.02 to 0.6, from 0.02 to 0.5, from 0.02 to 0.45, from 0.02 to 0.4, from 0.02 to 0.35, from 0.02 to 0.3, from 0.02 to 0.25, from 0.02 to 0.2, from 0.02 to 0.19, from 0.02 to 0.18, from 0.02 to 0.17, from 0.02 to 0.16, from 0.02 to 0.15, from 0.02 to 0.14, from 0.02 to 0.13, from 0.02 to 0.12, from 0.02 to 0.11, from 0.02 to 0.1, from 0.03 to 0.9, from 0.03 to 0.8, from 0.03 to 0.7, from 0.03 to 0.6, from 0.03 to 0.5, from 0.03 to 0.45, from 0.03 to 0.4, from 0.03 to 0.35, from 0.03 to 0.3, from 0.03 to 0.25, from 0.03 to 0.2, from 0.03 to 0.19, from 0.03 to 0.18, from 0.03 to 0.17, from 0.03 to 0.16, from 0.03 to 0.15, from 0.03 to 0.14, from 0.03 to 0.13, from 0.03 to 0.12, from 0.03 to 0.11, from 0.03 to 0.1, from 0.04 to 0.9, from 0.04 to 0.8, from 0.04 to 0.7, from 0.04 to 0.6, from 0.04 to 0.5, from 0.04 to 0.45, from 0.04 to 0.4, from 0.04 to 0.35, from 0.04 to 0.3, from 0.04 to 0.25, from 0.04 to 0.2, from 0.04 to 0.19, from 0.04 to 0.18, from 0.04 to 0.17, from 0.04 to 0.16, from 0.04 to 0.15, from 0.04 to 0.14, from 0.04 to 0.13, from 0.04 to 0.12, from 0.04 to 0.11, from 0.04 to 0.1, from 0.05 to 0.9, from 0.05 to 0.8, from 0.05 to 0.7, from 0.05 to 0.6, from 0.05 to 0.5, from 0.05 to 0.45, from 0.05 to 0.4, from 0.05 to 0.35, from 0.05 to 0.3, from 0.05 to 0.25, from 0.05 to 0.2, from 0.05 to 0.19, from 0.05 to 0.18, from 0.05 to 0.17, from 0.05 to 0.16, from 0.05 to 0.15, from 0.05 to 0.14, from 0.05 to 0.13, from 0.05 to 0.12, from 0.05 to 0.11, from 0.05 to 0.1, from 0.06 to 0.9, from 0.06 to 0.8, from 0.06 to 0.7, from 0.06 to 0.6, from 0.06 to 0.5, from 0.06 to 0.45, from 0.06 to 0.4, from 0.06 to 0.35, from 0.06 to 0.3, from 0.06 to 0.25, from 0.06 to 0.2, from 0.06 to 0.19, from 0.06 to 0.18, from 0.06 to 0.17, from 0.06 to 0.16, from 0.06 to 0.15, from 0.06 to 0.14, from 0.06 to 0.13, from 0.06 to 0.12, from 0.06 to 0.11, from 0.06 to 0.1, from 0.07 to 0.9, from 0.07 to 0.8, from 0.07 to 0.7, from 0.07 to 0.6, from 0.07 to 0.5, from 0.07 to 0.45, from 0.07 to 0.4, from 0.07 to 0.35, from 0.07 to 0.3, from 0.07 to 0.25, from 0.07 to 0.2, from 0.07 to 0.19, from 0.07 to 0.18, from 0.07 to 0.17, from 0.07 to 0.16, from 0.07 to 0.15, from 0.07 to 0.14, from 0.07 to 0.13, from 0.07 to 0.12, from 0.07 to 0.11, from 0.07 to 0.1, from 0.08 to 0.9, from 0.08 to 0.8, from 0.08 to 0.7, from 0.08 to 0.6, from 0.08 to 0.5, from 0.08 to 0.45, from 0.08 to 0.4, from 0.08 to 0.35, from 0.08 to 0.3, from 0.08 to 0.25, from 0.08 to 0.2, from 0.08 to 0.19, from 0.08 to 0.18, from 0.08 to 0.17, from 0.08 to 0.16, from 0.08 to 0.15, from 0.08 to 0.14, from 0.08 to 0.13, from 0.08 to 0.12, from 0.08 to 0.11, from 0.08 to 0.1, from 0.09 to 0.9, from 0.09 to 0.8, from 0.09 to 0.7, from 0.09 to 0.6, from 0.09 to 0.5, from 0.09 to 0.45, from 0.09 to 0.4, from 0.09 to 0.35, from 0.09 to 0.3, from 0.09 to 0.25, from 0.09 to 0.2, from 0.09 to 0.19, from 0.09 to 0.18, from 0.09 to 0.17, from 0.09 to 0.16, from 0.09 to 0.15, from 0.09 to 0.14, from 0.09 to 0.13, from 0.09 to 0.12, from 0.09 to 0.11, from 0.09 to 0.1, from 0.1 to 0.9, from 0.1 to 0.8, from 0.1 to 0.7, from 0.1 to 0.6, from 0.1 to 0.5, from 0.1 to 0.45, from 0.1 to 0.4, from 0.1 to 0.35, from 0.1 to 0.3, from 0.1 to 0.25, from 0.1 to 0.2, from 0.1 to 0.19, from 0.1 to 0.18, from 0.1 to 0.17, from 0.1 to 0.16, from 0.1 to 0.15, from 0.1 to 0.14, from 0.1 to 0.13, from 0.1 to 0.12, or from 0.1 to 0.11) equivalents, based on the amount of polyisobutylene being reacted.

In certain embodiments, the total amount of ethylenically unsaturated acylating agent added during the processes to produce the radically-functionalized PIBSA product described herein may be from 0.5 to 3 (such as from 0.5 to 2.9, from 0.5 to 2.8, from 0.5 to 2.7, from 0.5 to 2.6, from 0.5 to 2.5, from 0.5 to 2.4, from 0.5 to 2.3, from 0.5 to 2.4, from 0.5 to 2.3, from 0.5 to 2.2, from 0.5 to 2.1, from 0.5 to 2, from 0.5 to 1.9, from 0.5 to 1.8, from 0.5 to 1.7, from 0.5 to 1.6, from 0.5 to 1.5, from 0.5 to 1.4, from 0.5 to 1.3, from 0.5 to 1.2, from 0.5 to 1.1, from 0.5 to 1, from 0.6 to 3, from 0.6 to 2.9, from 0.6 to 2.8, from 0.6 to 2.7, from 0.6 to 2.6, from 0.6 to 2.5, from 0.6 to 2.4, from 0.6 to 2.3, from 0.6 to 2.4, from 0.6 to 2.3, from 0.6 to 2.2, from 0.6 to 2.1, from 0.6 to 2, from 0.6 to 1.9, from 0.6 to 1.8, from 0.6 to 1.7, from 0.6 to 1.6, from 0.6 to 1.5, from 0.6 to 1.4, from 0.6 to 1.3, from 0.6 to 1.2, from 0.6 to 1.1, from 0.6 to 1, from 0.7 to 3, from 0.7 to 2.9, from 0.7 to 2.8, from 0.7 to 2.7, from 0.7 to 2.6, from 0.7 to 2.5, from 0.7 to 2.4, from 0.7 to 2.3, from 0.7 to 2.4, from 0.7 to 2.3, from 0.7 to 2.2, from 0.7 to 2.1, from 0.7 to 2, from 0.7 to 1.9, from 0.7 to 1.8, from 0.7 to 1.7, from 0.7 to 1.6, from 0.7 to 1.5, from 0.7 to 1.4, from 0.7 to 1.3, from 0.7 to 1.2, from 0.7 to 1.1, from 0.7 to 1, from 0.8 to 3, from 0.8 to 2.9, from 0.8 to 2.8, from 0.8 to 2.7, from 0.8 to 2.6, from 0.8 to 2.5, from 0.8 to 2.4, from 0.8 to 2.3, from 0.8 to 2.4, from 0.8 to 2.3, from 0.8 to 2.2, from 0.8 to 2.1, from 0.8 to 2, from 0.8 to 1.9, from 0.8 to 1.8, from 0.8 to 1.7, from 0.8 to 1.6, from 0.8 to 1.5, from 0.8 to 1.4, from 0.8 to 1.3, from 0.8 to 1.2, from 0.8 to 1.1, from 0.8 to 1, from 0.9 to 3, from 0.9 to 2.9, from 0.9 to 2.8, from 0.9 to 2.7, from 0.9 to 2.6, from 0.9 to 2.5, from 0.9 to 2.4, from 0.9 to 2.3, from 0.9 to 2.2, from 0.9 to 2.1, from 0.9 to 2, from 0.9 to 1.9, from 0.9 to 1.8, from 0.9 to 1.7, from 0.9 to 1.6, from 0.9 to 1.5, from 0.9 to 1.4, from 0.9 to 1.3, from 0.9 to 1.2, from 0.9 to 1.1, from 0.9 to 1, from 1 to 3, from 1 to 2.9, from 1 to 2.8, from 1 to 2.7, from 1 to 2.6, from 1 to 2.5, from 1 to 2.4, from 1 to 2.3, from 1 to 2.2, from 1 to 2.1, from 1 to 2, from 1 to 1.9, from 1 to 1.8, from 1 to 1.7, from 1 to 1.6, from 1 to 1.5, from 1 to 1.4, from 1 to 1.3, from 1 to 1.2, from 1 to 1.1, from 1.1 to 3, from 1.1 to 2.9, from 1.1 to 2.8, from 1.1 to 2.7, from 1.1 to 2.6, from 1.1 to 2.5, from 1.1 to 2.4, from 1.1 to 2.3, from 1.1 to 2.2, from 1.1 to 2.1, from 1.1 to 2, from 1.1 to 1.9, from 1.1 to 1.8, from 1.1 to 1.7, from 1.1 to 1.6, from 1.1 to 1.5, from 1.1 to 1.4, from 1.1 to 1.3, from 1.1 to 1.2, from 1.2 to 3, from 1.2 to 2.9, from 1.2 to 2.8, from 1.2 to 2.7, from 1.2 to 2.6, from 1.2 to 2.5, from 1.2 to 2.4, from 1.2 to 2.3, from 1.2 to 2.2, from 1.2 to 2.1, from 1.2 to 2, from 1.2 to 1.9, from 1.2 to 1.8, from 1.2 to 1.7, from 1.2 to 1.6, from 1.2 to 1.5, from 1.2 to 1.4, from 1.2 to 1.3, from 1.3 to 3, from 1.3 to 2.9, from 1.3 to 2.8, from 1.3 to 2.7, from 1.3 to 2.6, from 1.3 to 2.5, from 1.3 to 2.4, from 1.3 to 2.3, from 1.3 to 2.2, from 1.3 to 2.1, from 1.3 to 2, from 1.3 to 1.9, from 1.3 to 1.8, from 1.3 to 1.7, from 1.3 to 1.6, from 1.3 to 1.5, from 1.3 to 1.4, from 1.4 to 3, from 1.4 to 2.9, from 1.4 to 2.8, from 1.4 to 2.7, from 1.4 to 2.6, from 1.4 to 2.5, from 1.4 to 2.4, from 1.4 to 2.3, from 1.4 to 2.2, from 1.4 to 2.1, from 1.4 to 2, from 1.4 to 1.9, from 1.4 to 1.8, from 1.4 to 1.7, from 1.4 to 1.6, from 1.4 to 1.5, from 1.5 to 3, from 1.5 to 2.9, from 1.5 to 2.8, from 1.5 to 2.7, from 1.5 to 2.6, from 1.5 to 2.5, from 1.5 to 2.4, from 1.5 to 2.3, from 1.5 to 2.2, from 1.5 to 2.1, from 1.5 to 2, from 1.5 to 1.9, from 1.5 to 1.8, from 1.5 to 1.7, from 1.5 to 1.6, from 1.6 to 3, from 1.6 to 2.9, from 1.6 to 2.8, from 1.6 to 2.7, from 1.6 to 2.6, from 1.6 to 2.5, from 1.6 to 2.4, from 1.6 to 2.3, from 1.6 to 2.2, from 1.6 to 2.1, from 1.6 to 2, from 1.6 to 1.9, from 1.6 to 1.8, from 1.6 to 1.7, from 1.7 to 3, from 1.7 to 2.9, from 1.7 to 2.8, from 1.7 to 2.7, from 1.7 to 2.6, from 1.7 to 2.5, from 1.7 to 2.4, from 1.7 to 2.3, from 1.7 to 2.2, from 1.7 to 2.1, from 1.7 to 2, from 1.7 to 1.9, from 1.7 to 1.8, from 1.8 to 3, from 1.8 to 2.9, from 1.8 to 2.8, from 1.8 to 2.7, from 1.8 to 2.6, from 1.8 to 2.5, from 1.8 to 2.4, from 1.8 to 2.3, from 1.8 to 2.2, from 1.8 to 2.1, from 1.8 to 2, from 1.8 to 1.9, from 1.9 to 3, from 1.9 to 2.9, from 1.9 to 2.8, from 1.9 to 2.7, from 1.9 to 2.6, from 1.9 to 2.5, from 1.9 to 2.4, from 1.9 to 2.3, from 1.9 to 2.2, from 1.9 to 2.1, from 1.9 to 2, from 2 to 3, from 2 to 2.9, from 2 to 2.8, from 2 to 2.7, from 2 to 2.6, from 2 to 2.5, from 2 to 2.4, from 2 to 2.3, from 2 to 2.2, from 2 to 2.1, from 2.1 to 3, from 2.1 to 2.9, from 2.1 to 2.8, from 2.1 to 2.7, from 2.1 to 2.6, from 2.1 to 2.5, from 2.1 to 2.4, from 2.1 to 2.3, from 2.1 to 2.2, from 2.2 to 3, from 2.2 to 2.9, from 2.2 to 2.8, from 2.2 to 2.7, from 2.2 to 2.6, from 2.2 to 2.5, from 2.2 to 2.4, from 2.2 to 2.3, from 2.3 to 3, from 2.3 to 2.9, from 2.3 to 2.8, from 2.3 to 2.7, from 2.3 to 2.6, from 2.3 to 2.5, from 2.3 to 2.4, from 2.4 to 3, from 2.4 to 2.9, from 2.4 to 2.8, from 2.4 to 2.7, from 2.4 to 2.6, from 2.4 to 2.5, from 2.5 to 3, from 2.5 to 2.9, from 2.5 to 2.8, from 2.5 to 2.7, or from 2.5 to 2.6) equivalents, based on the amount of polyisobutylene being reacted.

In certain embodiments, the radically-functionalized PIBSA product comprises less than 15 (such as less than 14, 13, 12, 11, 10, 9, 8, 7, 6, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) weight percent of a material comprising two or more polyisobutylene units linked by one or more acylating agent units. In certain embodiments, the radically-functionalized PIBSA product is substantially free of a material comprising two or more polyisobutylene units linked by one or more acylating agent units. In certain embodiments, the radically-functionalized PIBSA product comprises from 0 (such as greater than 0) to 15 (such as 14, 13, 12, 11, 10, 9, 8, 7, 6, 5, 4.5, 4, 3.5, 3, 2.5, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) weight percent of a material comprising two or more polyisobutylene units linked by one or more acylating agent units. In certain embodiments, the radically-functionalized PIBSA product comprises from 0 to 15 (such as from 0 to 14, from 0 to 13, from 0 to 12, from 0 to 11, from 0 to 10, from 0 to 9, from 0 to 8, from 0 to 7, from 0 to 6, from 0 to 5, from 0 to 4.5, from 0 to 4, from 0 to 3.5, from 0 to 3, from 0 to 2.5, from 0 to 2, from 0 to 1.9, from 0 to 1.8, from 0 to 1.7, from 0 to 1.6, from 0 to 1.5, from 0 to 1.4, from 0 to 1.3, from 0 to 1.2, from 0 to 1.1, from 0 to 1, from 0 to 0.9, from 0 to 0.8, from 0 to 0.7, from 0 to 0.6, from 0 to 0.5, from 0 to 0.4, from 0 to 0.3, from 0 to 0.2, from 0 to 0.1, from greater than 0 to 15, from greater than 0 to 14, from greater than 0 to 13, from greater than 0 to 12, from greater than 0 to 11, from greater than 0 to 10, from greater than 0 to 9, from greater than 0 to 8, from greater than 0 to 7, from greater than 0 to 6, from greater than 0 to 5, from greater than 0 to 4.5, from greater than 0 to 4, from greater than 0 to 3.5, from greater than 0 to 3, from greater than 0 to 2.5, from greater than 0 to 2, from greater than 0 to 1.9, from greater than 0 to 1.8, from greater than 0 to 1.7, from greater than 0 to 1.6, from greater than 0 to 1.5, from greater than 0 to 1.4, from greater than 0 to 1.3, from greater than 0 to 1.2, from greater than 0 to 1.1, from greater than 0 to 1, from greater than 0 to 0.9, from greater than 0 to 0.8, from greater than 0 to 0.7, from greater than 0 to 0.6, from greater than 0 to 0.5, from greater than 0 to 0.4, from greater than 0 to 0.3, from greater than 0 to 0.2, from greater than 0 to 0.1, from 0.1 to 15, from 0.1 to 14, from 0.1 to 13, from 0.1 to 12, from 0.1 to 11, from 0.1 to 10, from 0.1 to 9, from 0.1 to 8, from 0.1 to 7, from 0.1 to 6, from 0.1 to 5, from 0.1 to 4.5, from 0.1 to 4, from 0.1 to 3.5, from 0.1 to 3, from 0.1 to 2.5, from 0.1 to 2, from 0.1 to 1.9, from 0.1 to 1.8, from 0.1 to 1.7, from 0.1 to 1.6, from 0.1 to 1.5, from 0.1 to 1.4, from 0.1 to 1.3, from 0.1 to 1.2, from 0.1 to 1.1, from 0.1 to 1, from 0.1 to 0.9, from 0.1 to 0.8, from 0.1 to 0.7, from 0.1 to 0.6, from 0.1 to 0.5, from 0.1 to 0.4, from 0.1 to 0.3, from 0.1 to 0.2, from 0.2 to 15, from 0.2 to 14, from 0.2 to 13, from 0.2 to 12, from 0.2 to 11, from 0.2 to 10, from 0.2 to 9, from 0.2 to 8, from 0.2 to 7, from 0.2 to 6, from 0.2 to 5, from 0.2 to 4.5, from 0.2 to 4, from 0.2 to 3.5, from 0.2 to 3, from 0.2 to 2.5, from 0.2 to 2, from 0.2 to 1.9, from 0.2 to 1.8, from 0.2 to 1.7, from 0.2 to 1.6, from 0.2 to 1.5, from 0.2 to 1.4, from 0.2 to 1.3, from 0.2 to 1.2, from 0.2 to 1.1, from 0.2 to 1, from 0.2 to 0.9, from 0.2 to 0.8, from 0.2 to 0.7, from 0.2 to 0.6, from 0.2 to 0.5, from 0.2 to 0.4, from 0.2 to 0.3, from 0.3 to 15, from 0.3 to 14, from 0.3 to 13, from 0.3 to 12, from 0.3 to 11, from 0.3 to 10, from 0.3 to 9, from 0.3 to 8, from 0.3 to 7, from 0.3 to 6, from 0.3 to 5, from 0.3 to 4.5, from 0.3 to 4, from 0.3 to 3.5, from 0.3 to 3, from 0.3 to 2.5, from 0.3 to 2, from 0.3 to 1.9, from 0.3 to 1.8, from 0.3 to 1.7, from 0.3 to 1.6, from 0.3 to 1.5, from 0.3 to 1.4, from 0.3 to 1.3, from 0.3 to 1.2, from 0.3 to 1.1, from 0.3 to 1, from 0.3 to 0.9, from 0.3 to 0.8, from 0.3 to 0.7, from 0.3 to 0.6, from 0.3 to 0.5, from 0.3 to 0.4, from 0.4 to 15, from 0.4 to 14, from 0.4 to 13, from 0.4 to 12, from 0.4 to 11, from 0.4 to 10, from 0.4 to 9, from 0.4 to 8, from 0.4 to 7, from 0.4 to 6, from 0.4 to 5, from 0.4 to 4.5, from 0.4 to 4, from 0.4 to 3.5, from 0.4 to 3, from 0.4 to 2.5, from 0.4 to 2, from 0.4 to 1.9, from 0.4 to 1.8, from 0.4 to 1.7, from 0.4 to 1.6, from 0.4 to 1.5, from 0.4 to 1.4, from 0.4 to 1.3, from 0.4 to 1.2, from 0.4 to 1.1, from 0.4 to 1, from 0.4 to 0.9, from 0.4 to 0.8, from 0.4 to 0.7, from 0.4 to 0.6, from 0.4 to 0.5, from 0.5 to 15, from 0.5 to 14, from 0.5 to 13, from 0.5 to 12, from 0.5 to 11, from 0.5 to 10, from 0.5 to 9, from 0.5 to 8, from 0.5 to 7, from 0.5 to 6, from 0.5 to 5, from 0.5 to 4.5, from 0.5 to 4, from 0.5 to 3.5, from 0.5 to 3, from 0.5 to 2.5, from 0.5 to 2, from 0.5 to 1.9, from 0.5 to 1.8, from 0.5 to 1.7, from 0.5 to 1.6, from 0.5 to 1.5, from 0.5 to 1.4, from 0.5 to 1.3, from 0.5 to 1.2, from 0.5 to 1.1, from 0.5 to 1, from 0.5 to 0.9, from 0.5 to 0.8, from 0.5 to 0.7, from 0.5 to 0.6, from 0.6 to 15, from 0.6 to 14, from 0.6 to 13, from 0.6 to 12, from 0.6 to 11, from 0.6 to 10, from 0.6 to 9, from 0.6 to 8, from 0.6 to 7, from 0.6 to 6, from 0.6 to 5, from 0.6 to 4.5, from 0.6 to 4, from 0.6 to 3.5, from 0.6 to 3, from 0.6 to 2.5, from 0.6 to 2, from 0.6 to 1.9, from 0.6 to 1.8, from 0.6 to 1.7, from 0.6 to 1.6, from 0.6 to 1.5, from 0.6 to 1.4, from 0.6 to 1.3, from 0.6 to 1.2, from 0.6 to 1.1, from 0.6 to 1, from 0.6 to 0.9, from 0.6 to 0.8, from 0.6 to 0.7, from 0.7 to 15, from 0.7 to 14, from 0.7 to 13, from 0.7 to 12, from 0.7 to 11, from 0.7 to 10, from 0.7 to 9, from 0.7 to 8, from 0.7 to 7, from 0.7 to 6, from 0.7 to 5, from 0.7 to 4.5, from 0.7 to 4, from 0.7 to 3.5, from 0.7 to 3, from 0.7 to 2.5, from 0.7 to 2, from 0.7 to 1.9, from 0.7 to 1.8, from 0.7 to 1.7, from 0.7 to 1.6, from 0.7 to 1.5, from 0.7 to 1.4, from 0.7 to 1.3, from 0.7 to 1.2, from 0.7 to 1.1, from 0.7 to 1, from 0.7 to 0.9, from 0.7 to 0.8, from 0.8 to 15, from 0.8 to 14, from 0.8 to 13, from 0.8 to 12, from 0.8 to 11, from 0.8 to 10, from 0.8 to 9, from 0.8 to 8, from 0.8 to 7, from 0.8 to 6, from 0.8 to 5, from 0.8 to 4.5, from 0.8 to 4, from 0.8 to 3.5, from 0.8 to 3, from 0.8 to 2.5, from 0.8 to 2, from 0.8 to 1.9, from 0.8 to 1.8, from 0.8 to 1.7, from 0.8 to 1.6, from 0.8 to 1.5, from 0.8 to 1.4, from 0.8 to 1.3, from 0.8 to 1.2, from 0.8 to 1.1, from 0.8 to 1, from 0.8 to 0.9, from 0.9 to 15, from 0.9 to 14, from 0.9 to 13, from 0.9 to 12, from 0.9 to 11, from 0.9 to 10, from 0.9 to 9, from 0.9 to 8, from 0.9 to 7, from 0.9 to 6, from 0.9 to 5, from 0.9 to 4.5, from 0.9 to 4, from 0.9 to 3.5, from 0.9 to 3, from 0.9 to 2.5, from 0.9 to 2, from 0.9 to 1.9, from 0.9 to 1.8, from 0.9 to 1.7, from 0.9 to 1.6, from 0.9 to 1.5, from 0.9 to 1.4, from 0.9 to 1.3, from 0.9 to 1.2, from 0.9 to 1.1, from 0.9 to 1, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4.5, from 1 to 4, from 1 to 3.5, from 1 to 3, from 1 to 2.5, from 1 to 2, from 1 to 1.9, from 1 to 1.8, from 1 to 1.7, from 1 to 1.6, from 1 to 1.5, from 1 to 1.4, from 1 to 1.3, from 1 to 1.2, from 1 to 1.1, from 1.1 to 15, from 1.1 to 14, from 1.1 to 13, from 1.1 to 12, from 1.1 to 11, from 1.1 to 10, from 1.1 to 9, from 1.1 to 8, from 1.1 to 7, from 1.1 to 6, from 1.1 to 5, from 1.1 to 4.5, from 1.1 to 4, from 1.1 to 3.5, from 1.1 to 3, from 1.1 to 2.5, from 1.1 to 2, from 1.1 to 1.9, from 1.1 to 1.8, from 1.1 to 1.7, from 1.1 to 1.6, from 1.1 to 1.5, from 1.1 to 1.4, from 1.1 to 1.3, from 1.1 to 1.2, from 1.2 to 15, from 1.2 to 14, from 1.2 to 13, from 1.2 to 12, from 1.2 to 11, from 1.2 to 10, from 1.2 to 9, from 1.2 to 8, from 1.2 to 7, from 1.2 to 6, from 1.2 to 5, from 1.2 to 4.5, from 1.2 to 4, from 1.2 to 3.5, from 1.2 to 3, from 1.2 to 2.5, from 1.2 to 2, from 1.2 to 1.9, from 1.2 to 1.8, from 1.2 to 1.7, from 1.2 to 1.6, from 1.2 to 1.5, from 1.2 to 1.4, from 1.2 to 1.3, from 1.3 to 15, from 1.3 to 14, from 1.3 to 13, from 1.3 to 12, from 1.3 to 11, from 1.3 to 10, from 1.3 to 9, from 1.3 to 8, from 1.3 to 7, from 1.3 to 6, from 1.3 to 5, from 1.3 to 4.5, from 1.3 to 4, from 1.3 to 3.5, from 1.3 to 3, from 1.3 to 2.5, from 1.3 to 2, from 1.3 to 1.9, from 1.3 to 1.8, from 1.3 to 1.7, from 1.3 to 1.6, from 1.3 to 1.5, from 1.3 to 1.4, from 1.4 to 15, from 1.4 to 14, from 1.4 to 13, from 1.4 to 12, from 1.4 to 11, from 1.4 to 10, from 1.4 to 9, from 1.4 to 8, from 1.4 to 7, from 1.4 to 6, from 1.4 to 5, from 1.4 to 4.5, from 1.4 to 4, from 1.4 to 3.5, from 1.4 to 3, from 1.4 to 2.5, from 1.4 to 2, from 1.4 to 1.9, from 1.4 to 1.8, from 1.4 to 1.7, from 1.4 to 1.6, from 1.4 to 1.5, from 1.5 to 15, from 1.5 to 14, from 1.5 to 13, from 1.5 to 12, from 1.5 to 11, from 1.5 to 10, from 1.5 to 9, from 1.5 to 8, from 1.5 to 7, from 1.5 to 6, from 1.5 to 5, from 1.5 to 4.5, from 1.5 to 4, from 1.5 to 3.5, from 1.5 to 3, from 1.5 to 2.5, from 1.5 to 2, from 1.5 to 1.9, from 1.5 to 1.8, from 1.5 to 1.7, from 1.5 to 1.6, from 1.6 to 15, from 1.6 to 14, from 1.6 to 13, from 1.6 to 12, from 1.6 to 11, from 1.6 to 10, from 1.6 to 9, from 1.6 to 8, from 1.6 to 7, from 1.6 to 6, from 1.6 to 5, from 1.6 to 4.5, from 1.6 to 4, from 1.6 to 3.5, from 1.6 to 3, from 1.6 to 2.5, from 1.6 to 2, from 1.6 to 1.9, from 1.6 to 1.8, from 1.6 to 1.7, from 1.7 to 15, from 1.7 to 14, from 1.7 to 13, from 1.7 to 12, from 1.7 to 11, from 1.7 to 10, from 1.7 to 9, from 1.7 to 8, from 1.7 to 7, from 1.7 to 6, from 1.7 to 5, from 1.7 to 4.5, from 1.7 to 4, from 1.7 to 3.5, from 1.7 to 3, from 1.7 to 2.5, from 1.7 to 2, from 1.7 to 1.9, from 1.7 to 1.8, from 1.8 to 15, from 1.8 to 14, from 1.8 to 13, from 1.8 to 12, from 1.8 to 11, from 1.8 to 10, from 1.8 to 9, from 1.8 to 8, from 1.8 to 7, from 1.8 to 6, from 1.8 to 5, from 1.8 to 4.5, from 1.8 to 4, from 1.8 to 3.5, from 1.8 to 3, from 1.8 to 2.5, from 1.8 to 2, from 1.8 to 1.9, from 1.9 to 15, from 1.9 to 14, from 1.9 to 13, from 1.9 to 12, from 1.9 to 11, from 1.9 to 10, from 1.9 to 9, from 1.9 to 8, from 1.9 to 7, from 1.9 to 6, from 1.9 to 5, from 1.9 to 4.5, from 1.9 to 4, from 1.9 to 3.5, from 1.9 to 3, from 1.9 to 2.5, from 1.9 to 2, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4.5, from 2 to 4, from 2 to 3.5, from 2 to 3, from 2 to 2.5, from 2.5 to 15, from 2.5 to 14, from 2.5 to 13, from 2.5 to 12, from 2.5 to 11, from 2.5 to 10, from 2.5 to 9, from 2.5 to 8, from 2.5 to 7, from 2.5 to 6, from 2.5 to 5, from 2.5 to 4.5, from 2.5 to 4, from 2.5 to 3.5, from 2.5 to 3, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4.5, from 3 to 4, from 3 to 3.5, from 3.5 to 15, from 3.5 to 14, from 3.5 to 13, from 3.5 to 12, from 3.5 to 11, from 3.5 to 10, from 3.5 to 9, from 3.5 to 8, from 3.5 to 7, from 3.5 to 6, from 3.5 to 5, from 3.5 to 4.5, from 3.5 to 4, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 4 to 4.5, from 4.5 to 15, from 4.5 to 14, from 4.5 to 13, from 4.5 to 12, from 4.5 to 11, from 4.5 to 10, from 4.5 to 9, from 4.5 to 8, from 4.5 to 7, from 4.5 to 6, from 4.5 to 5, from 5 to 15, from 5 to 14, from 5 to 13, from 5 to 12, from 5 to 11, from 5 to 10, from 5 to 9, from 5 to 8, from 5 to 7, from 5 to 6, from 6 to 15, from 6 to 14, from 6 to 13, from 6 to 12, from 6 to 11, from 6 to 10, from 6 to 9, from 6 to 8, from 6 to 7, from 7 to 15, from 7 to 14, from 7 to 13, from 7 to 12, from 7 to 11, from 7 to 10, from 7 to 9, from 7 to 8, from 8 to 15, from 8 to 14, from 8 to 13, from 8 to 12, from 8 to 11, from 8 to 10, from 8 to 9, from 9 to 15, from 9 to 14, from 9 to 13, from 9 to 12, from 9 to 11, from 9 to 10, from 10 to 15, from 10 to 14, from 10 to 13, from 10 to 12, from 10 to 11, from 11 to 15, from 11 to 14, from 11 to 13, from 11 to 12, from 12 to 15, from 12 to 14, from 12 to 13, from 13 to 15, from 13 to 14, or from 14 to 15) weight percent of a material comprising two or more polyisobutylene units linked by one or more acylating agent units.

In certain embodiments, the polyisobutylene comprises an average of at least 4 isobutylene monomeric units per molecule. In certain embodiments, the polyisobutylene comprises an average of 3 to 56 isobutylene monomeric units per molecule. In certain embodiments, the polyisobutylene comprises an average of 3 to 55 isobutylene monomeric units per molecule. In certain embodiments, the polyisobutylene comprises an average of 3 to 54 isobutylene monomeric units per molecule. In certain embodiments, the polyisobutylene comprises an average of 3 to 53 isobutylene monomeric units per molecule. In certain embodiments, the polyisobutylene comprises an average of 3 to 52 isobutylene monomeric units per molecule. In certain embodiments, the polyisobutylene comprises an average of 3 to 51 isobutylene monomeric units per molecule. In certain embodiments, the polyisobutylene comprises an average of 3 to 50 isobutylene monomeric units per molecule. In certain embodiments, the polyisobutylene comprises an average of 3 to 48 isobutylene monomeric units per molecule. In certain embodiments, the polyisobutylene comprises an average of 3 to 46 isobutylene monomeric units per molecule. In certain embodiments, the polyisobutylene comprises an average of 3 to 44 isobutylene monomeric units per molecule. In certain embodiments, the polyisobutylene comprises an average of 3 to 42 isobutylene monomeric units per molecule. In certain embodiments, the polyisobutylene comprises an average of 3 to 40 isobutylene monomeric units per molecule. In certain embodiments, the polyisobutylene comprises an average of 3 to 38 isobutylene monomeric units per molecule. In certain embodiments, the polyisobutylene comprises an average of 3 to 36 isobutylene monomeric units per molecule. In certain embodiments, the polyisobutylene comprises an average of 3 to 34 isobutylene monomeric units per molecule. In certain embodiments, the polyisobutylene comprises an average of 3 to 32 isobutylene monomeric units per molecule. In certain embodiments, the polyisobutylene comprises an average of 3 to 30 isobutylene monomeric units per molecule. In certain embodiments, the polyisobutylene comprises an average of 3 to 28 isobutylene monomeric units per molecule. In certain embodiments, the polyisobutylene comprises an average of 3 to 26 isobutylene monomeric units per molecule. In certain embodiments, the polyisobutylene comprises an average of 3 to 24 isobutylene monomeric units per molecule. In certain embodiments, the polyisobutylene comprises an average of 3 to 22 isobutylene monomeric units per molecule. In certain embodiments, the polyisobutylene comprises an average of 3 to 20 isobutylene monomeric units per molecule. In certain embodiments, the polyisobutylene comprises an average of 3 to 18 isobutylene monomeric units per molecule. In certain embodiments, the polyisobutylene comprises an average of 3 to 16 isobutylene monomeric units per molecule. In certain embodiments, the polyisobutylene comprises an average of 3 to 14 isobutylene monomeric units per molecule. In certain embodiments, the polyisobutylene comprises an average of 3 to 12 isobutylene monomeric units per molecule. In certain embodiments, the polyisobutylene comprises an average of 3 to 10 isobutylene monomeric units per molecule. In certain embodiments, the polyisobutylene comprises an average of 3 to 9 isobutylene monomeric units per molecule. In certain embodiments, the polyisobutylene comprises an average of 3 to 8 isobutylene monomeric units per molecule. In certain embodiments, the polyisobutylene comprises an average of 3 to 7 isobutylene monomeric units per molecule. In certain embodiments, the polyisobutylene comprises an average of 3 to 6 isobutylene monomeric units per molecule. In certain embodiments, the polyisobutylene comprises an average of 3 to 5 isobutylene monomeric units per molecule. In certain embodiments, the polyisobutylene comprises an average of 3 to 4 isobutylene monomeric units per molecule. In certain embodiments, the polyisobutylene comprises an average of 4 to 56 isobutylene monomeric units per molecule. In certain embodiments, the polyisobutylene comprises an average of 4 to 55 isobutylene monomeric units per molecule. In certain embodiments, the polyisobutylene comprises an average of 4 to 54 isobutylene monomeric units per molecule. In certain embodiments, the polyisobutylene comprises an average of 4 to 53 isobutylene monomeric units per molecule. In certain embodiments, the polyisobutylene comprises an average of 4 to 52 isobutylene monomeric units per molecule. In certain embodiments, the polyisobutylene comprises an average of 4 to 51 isobutylene monomeric units per molecule. In certain embodiments, the polyisobutylene comprises an average of 4 to 50 isobutylene monomeric units per molecule. In certain embodiments, the polyisobutylene comprises an average of 4 to 48 isobutylene monomeric units per molecule. In certain embodiments, the polyisobutylene comprises an average of 4 to 46 isobutylene monomeric units per molecule. In certain embodiments, the polyisobutylene comprises an average of 4 to 44 isobutylene monomeric units per molecule. In certain embodiments, the polyisobutylene comprises an average of 4 to 42 isobutylene monomeric units per molecule. In certain embodiments, the polyisobutylene comprises an average of 4 to 40 isobutylene monomeric units per molecule. In certain embodiments, the polyisobutylene comprises an average of 4 to 38 isobutylene monomeric units per molecule. In certain embodiments, the polyisobutylene comprises an average of 4 to 36 isobutylene monomeric units per molecule. In certain embodiments, the polyisobutylene comprises an average of 4 to 34 isobutylene monomeric units per molecule. In certain embodiments, the polyisobutylene comprises an average of 4 to 32 isobutylene monomeric units per molecule. In certain embodiments, the polyisobutylene comprises an average of 4 to 30 isobutylene monomeric units per molecule. In certain embodiments, the polyisobutylene comprises an average of 4 to 28 isobutylene monomeric units per molecule. In certain embodiments, the polyisobutylene comprises an average of 4 to 26 isobutylene monomeric units per molecule. In certain embodiments, the polyisobutylene comprises an average of 4 to 24 isobutylene monomeric units per molecule. In certain embodiments, the polyisobutylene comprises an average of 4 to 22 isobutylene monomeric units per molecule. In certain embodiments, the polyisobutylene comprises an average of 4 to 20 isobutylene monomeric units per molecule. In certain embodiments, the polyisobutylene comprises an average of 4 to 18 isobutylene monomeric units per molecule. In certain embodiments, the polyisobutylene comprises an average of 4 to 16 isobutylene monomeric units per molecule. In certain embodiments, the polyisobutylene comprises an average of 4 to 14 isobutylene monomeric units per molecule. In certain embodiments, the polyisobutylene comprises an average of 4 to 12 isobutylene monomeric units per molecule. In certain embodiments, the polyisobutylene comprises an average of 4 to 10 isobutylene monomeric units per molecule. In certain embodiments, the polyisobutylene comprises an average of 4 to 9 isobutylene monomeric units per molecule. In certain embodiments, the polyisobutylene comprises an average of 4 to 8 isobutylene monomeric units per molecule. In certain embodiments, the polyisobutylene comprises an average of 4 to 7 isobutylene monomeric units per molecule. In certain embodiments, the polyisobutylene comprises an average of 4 to 6 isobutylene monomeric units per molecule. In certain embodiments, the polyisobutylene comprises an average of 4 to 5 isobutylene monomeric units per molecule. In certain embodiments, the polyisobutylene comprises an average of 5 to 56 isobutylene monomeric units per molecule. In certain embodiments, the polyisobutylene comprises an average of 5 to 55 isobutylene monomeric units per molecule. In certain embodiments, the polyisobutylene comprises an average of 5 to 54 isobutylene monomeric units per molecule. In certain embodiments, the polyisobutylene comprises an average of 5 to 53 isobutylene monomeric units per molecule. In certain embodiments, the polyisobutylene comprises an average of 5 to 52 isobutylene monomeric units per molecule. In certain embodiments, the polyisobutylene comprises an average of 5 to 51 isobutylene monomeric units per molecule. In certain embodiments, the polyisobutylene comprises an average of 5 to 50 isobutylene monomeric units per molecule. In certain embodiments, the polyisobutylene comprises an average of 5 to 48 isobutylene monomeric units per molecule. In certain embodiments, the polyisobutylene comprises an average of 5 to 46 isobutylene monomeric units per molecule. In certain embodiments, the polyisobutylene comprises an average of 5 to 44 isobutylene monomeric units per molecule. In certain embodiments, the polyisobutylene comprises an average of 5 to 42 isobutylene monomeric units per molecule. In certain embodiments, the polyisobutylene comprises an average of 5 to 40 isobutylene monomeric units per molecule. In certain embodiments, the polyisobutylene comprises an average of 5 to 38 isobutylene monomeric units per molecule. In certain embodiments, the polyisobutylene comprises an average of 5 to 36 isobutylene monomeric units per molecule. In certain embodiments, the polyisobutylene comprises an average of 5 to 34 isobutylene monomeric units per molecule. In certain embodiments, the polyisobutylene comprises an average of 5 to 32 isobutylene monomeric units per molecule. In certain embodiments, the polyisobutylene comprises an average of 5 to 30 isobutylene monomeric units per molecule. In certain embodiments, the polyisobutylene comprises an average of 5 to 28 isobutylene monomeric units per molecule. In certain embodiments, the polyisobutylene comprises an average of 5 to 26 isobutylene monomeric units per molecule. In certain embodiments, the polyisobutylene comprises an average of 5 to 24 isobutylene monomeric units per molecule. In certain embodiments, the polyisobutylene comprises an average of 5 to 22 isobutylene monomeric units per molecule. In certain embodiments, the polyisobutylene comprises an average of 5 to 20 isobutylene monomeric units per molecule. In certain embodiments, the polyisobutylene comprises an average of 5 to 18 isobutylene monomeric units per molecule. In certain embodiments, the polyisobutylene comprises an average of 5 to 16 isobutylene monomeric units per molecule. In certain embodiments, the polyisobutylene comprises an average of 5 to 14 isobutylene monomeric units per molecule. In certain embodiments, the polyisobutylene comprises an average of 5 to 12 isobutylene monomeric units per molecule. In certain embodiments, the polyisobutylene comprises an average of 5 to 10 isobutylene monomeric units per molecule. In certain embodiments, the polyisobutylene comprises an average of 5 to 9 isobutylene monomeric units per molecule. In certain embodiments, the polyisobutylene comprises an average of 5 to 8 isobutylene monomeric units per molecule. In certain embodiments, the polyisobutylene comprises an average of 5 to 7 isobutylene monomeric units per molecule. In certain embodiments, the polyisobutylene comprises an average of 5 to 6 isobutylene monomeric units per molecule. In certain embodiments, the polyisobutylene comprises an average of 6 to 56 isobutylene monomeric units per molecule. In certain embodiments, the polyisobutylene comprises an average of 6 to 55 isobutylene monomeric units per molecule. In certain embodiments, the polyisobutylene comprises an average of 6 to 54 isobutylene monomeric units per molecule. In certain embodiments, the polyisobutylene comprises an average of 6 to 53 isobutylene monomeric units per molecule. In certain embodiments, the polyisobutylene comprises an average of 6 to 52 isobutylene monomeric units per molecule. In certain embodiments, the polyisobutylene comprises an average of 6 to 51 isobutylene monomeric units per molecule. In certain embodiments, the polyisobutylene comprises an average of 6 to 50 isobutylene monomeric units per molecule. In certain embodiments, the polyisobutylene comprises an average of 6 to 48 isobutylene monomeric units per molecule. In certain embodiments, the polyisobutylene comprises an average of 6 to 46 isobutylene monomeric units per molecule. In certain embodiments, the polyisobutylene comprises an average of 6 to 44 isobutylene monomeric units per molecule. In certain embodiments, the polyisobutylene comprises an average of 6 to 42 isobutylene monomeric units per molecule. In certain embodiments, the polyisobutylene comprises an average of 6 to 40 isobutylene monomeric units per molecule. In certain embodiments, the polyisobutylene comprises an average of 6 to 38 isobutylene monomeric units per molecule. In certain embodiments, the polyisobutylene comprises an average of 6 to 36 isobutylene monomeric units per molecule. In certain embodiments, the polyisobutylene comprises an average of 6 to 34 isobutylene monomeric units per molecule. In certain embodiments, the polyisobutylene comprises an average of 6 to 32 isobutylene monomeric units per molecule. In certain embodiments, the polyisobutylene comprises an average of 6 to 30 isobutylene monomeric units per molecule. In certain embodiments, the polyisobutylene comprises an average of 6 to 28 isobutylene monomeric units per molecule. In certain embodiments, the polyisobutylene comprises an average of 6 to 26 isobutylene monomeric units per molecule. In certain embodiments, the polyisobutylene comprises an average of 6 to 24 isobutylene monomeric units per molecule. In certain embodiments, the polyisobutylene comprises an average of 6 to 22 isobutylene monomeric units per molecule. In certain embodiments, the polyisobutylene comprises an average of 6 to 20 isobutylene monomeric units per molecule. In certain embodiments, the polyisobutylene comprises an average of 6 to 18 isobutylene monomeric units per molecule. In certain embodiments, the polyisobutylene comprises an average of 6 to 16 isobutylene monomeric units per molecule. In certain embodiments, the polyisobutylene comprises an average of 6 to 14 isobutylene monomeric units per molecule. In certain embodiments, the polyisobutylene comprises an average of 6 to 12 isobutylene monomeric units per molecule. In certain embodiments, the polyisobutylene comprises an average of 6 to 10 isobutylene monomeric units per molecule. In certain embodiments, the polyisobutylene comprises an average of 6 to 9 isobutylene monomeric units per molecule. In certain embodiments, the polyisobutylene comprises an average of 6 to 8 isobutylene monomeric units per molecule. In certain embodiments, the polyisobutylene comprises an average of 6 to 7 isobutylene monomeric units per molecule. In certain embodiments, the polyisobutylene comprises an average of 7 to 56 isobutylene monomeric units per molecule. In certain embodiments, the polyisobutylene comprises an average of 7 to 55 isobutylene monomeric units per molecule. In certain embodiments, the polyisobutylene comprises an average of 7 to 54 isobutylene monomeric units per molecule. In certain embodiments, the polyisobutylene comprises an average of 7 to 53 isobutylene monomeric units per molecule. In certain embodiments, the polyisobutylene comprises an average of 7 to 52 isobutylene monomeric units per molecule. In certain embodiments, the polyisobutylene comprises an average of 7 to 51 isobutylene monomeric units per molecule. In certain embodiments, the polyisobutylene comprises an average of 7 to 50 isobutylene monomeric units per molecule. In certain embodiments, the polyisobutylene comprises an average of 7 to 48 isobutylene monomeric units per molecule. In certain embodiments, the polyisobutylene comprises an average of 7 to 46 isobutylene monomeric units per molecule. In certain embodiments, the polyisobutylene comprises an average of 7 to 44 isobutylene monomeric units per molecule. In certain embodiments, the polyisobutylene comprises an average of 7 to 42 isobutylene monomeric units per molecule. In certain embodiments, the polyisobutylene comprises an average of 7 to 40 isobutylene monomeric units per molecule. In certain embodiments, the polyisobutylene comprises an average of 7 to 38 isobutylene monomeric units per molecule. In certain embodiments, the polyisobutylene comprises an average of 7 to 36 isobutylene monomeric units per molecule. In certain embodiments, the polyisobutylene comprises an average of 7 to 34 isobutylene monomeric units per molecule. In certain embodiments, the polyisobutylene comprises an average of 7 to 32 isobutylene monomeric units per molecule. In certain embodiments, the polyisobutylene comprises an average of 7 to 30 isobutylene monomeric units per molecule. In certain embodiments, the polyisobutylene comprises an average of 7 to 28 isobutylene monomeric units per molecule. In certain embodiments, the polyisobutylene comprises an average of 7 to 26 isobutylene monomeric units per molecule. In certain embodiments, the polyisobutylene comprises an average of 7 to 24 isobutylene monomeric units per molecule. In certain embodiments, the polyisobutylene comprises an average of 7 to 22 isobutylene monomeric units per molecule. In certain embodiments, the polyisobutylene comprises an average of 7 to 20 isobutylene monomeric units per molecule. In certain embodiments, the polyisobutylene comprises an average of 7 to 18 isobutylene monomeric units per molecule. In certain embodiments, the polyisobutylene comprises an average of 7 to 16 isobutylene monomeric units per molecule. In certain embodiments, the polyisobutylene comprises an average of 7 to 14 isobutylene monomeric units per molecule. In certain embodiments, the polyisobutylene comprises an average of 7 to 12 isobutylene monomeric units per molecule. In certain embodiments, the polyisobutylene comprises an average of 7 to 10 isobutylene monomeric units per molecule. In certain embodiments, the polyisobutylene comprises an average of 7 to 9 isobutylene monomeric units per molecule. In certain embodiments, the polyisobutylene comprises an average of 7 to 8 isobutylene monomeric units per molecule. In certain embodiments, the polyisobutylene comprises an average of 8 to 56 isobutylene monomeric units per molecule. In certain embodiments, the polyisobutylene comprises an average of 8 to 55 isobutylene monomeric units per molecule. In certain embodiments, the polyisobutylene comprises an average of 8 to 54 isobutylene monomeric units per molecule. In certain embodiments, the polyisobutylene comprises an average of 8 to 53 isobutylene monomeric units per molecule. In certain embodiments, the polyisobutylene comprises an average of 8 to 52 isobutylene monomeric units per molecule. In certain embodiments, the polyisobutylene comprises an average of 8 to 51 isobutylene monomeric units per molecule. In certain embodiments, the polyisobutylene comprises an average of 8 to 50 isobutylene monomeric units per molecule. In certain embodiments, the polyisobutylene comprises an average of 8 to 48 isobutylene monomeric units per molecule. In certain embodiments, the polyisobutylene comprises an average of 8 to 46 isobutylene monomeric units per molecule. In certain embodiments, the polyisobutylene comprises an average of 8 to 44 isobutylene monomeric units per molecule. In certain embodiments, the polyisobutylene comprises an average of 8 to 42 isobutylene monomeric units per molecule. In certain embodiments, the polyisobutylene comprises an average of 8 to 40 isobutylene monomeric units per molecule. In certain embodiments, the polyisobutylene comprises an average of 8 to 38 isobutylene monomeric units per molecule. In certain embodiments, the polyisobutylene comprises an average of 8 to 36 isobutylene monomeric units per molecule. In certain embodiments, the polyisobutylene comprises an average of 8 to 34 isobutylene monomeric units per molecule. In certain embodiments, the polyisobutylene comprises an average of 8 to 32 isobutylene monomeric units per molecule. In certain embodiments, the polyisobutylene comprises an average of 8 to 30 isobutylene monomeric units per molecule. In certain embodiments, the polyisobutylene comprises an average of 8 to 28 isobutylene monomeric units per molecule. In certain embodiments, the polyisobutylene comprises an average of 8 to 26 isobutylene monomeric units per molecule. In certain embodiments, the polyisobutylene comprises an average of 8 to 24 isobutylene monomeric units per molecule. In certain embodiments, the polyisobutylene comprises an average of 8 to 22 isobutylene monomeric units per molecule. In certain embodiments, the polyisobutylene comprises an average of 8 to 20 isobutylene monomeric units per molecule. In certain embodiments, the polyisobutylene comprises an average of 8 to 18 isobutylene monomeric units per molecule. In certain embodiments, the polyisobutylene comprises an average of 8 to 16 isobutylene monomeric units per molecule. In certain embodiments, the polyisobutylene comprises an average of 8 to 14 isobutylene monomeric units per molecule. In certain embodiments, the polyisobutylene comprises an average of 8 to 12 isobutylene monomeric units per molecule. In certain embodiments, the polyisobutylene comprises an average of 8 to 10 isobutylene monomeric units per molecule. In certain embodiments, the polyisobutylene comprises an average of 8 to 9 isobutylene monomeric units per molecule. In certain embodiments, the polyisobutylene comprises an average of 9 to 56 isobutylene monomeric units per molecule. In certain embodiments, the polyisobutylene comprises an average of 9 to 55 isobutylene monomeric units per molecule. In certain embodiments, the polyisobutylene comprises an average of 9 to 54 isobutylene monomeric units per molecule. In certain embodiments, the polyisobutylene comprises an average of 9 to 53 isobutylene monomeric units per molecule. In certain embodiments, the polyisobutylene comprises an average of 9 to 52 isobutylene monomeric units per molecule. In certain embodiments, the polyisobutylene comprises an average of 9 to 51 isobutylene monomeric units per molecule. In certain embodiments, the polyisobutylene comprises an average of 9 to 50 isobutylene monomeric units per molecule. In certain embodiments, the polyisobutylene comprises an average of 9 to 48 isobutylene monomeric units per molecule. In certain embodiments, the polyisobutylene comprises an average of 9 to 46 isobutylene monomeric units per molecule. In certain embodiments, the polyisobutylene comprises an average of 9 to 44 isobutylene monomeric units per molecule. In certain embodiments, the polyisobutylene comprises an average of 9 to 42 isobutylene monomeric units per molecule. In certain embodiments, the polyisobutylene comprises an average of 9 to 40 isobutylene monomeric units per molecule. In certain embodiments, the polyisobutylene comprises an average of 9 to 38 isobutylene monomeric units per molecule. In certain embodiments, the polyisobutylene comprises an average of 9 to 36 isobutylene monomeric units per molecule. In certain embodiments, the polyisobutylene comprises an average of 9 to 34 isobutylene monomeric units per molecule. In certain embodiments, the polyisobutylene comprises an average of 9 to 32 isobutylene monomeric units per molecule. In certain embodiments, the polyisobutylene comprises an average of 9 to 30 isobutylene monomeric units per molecule. In certain embodiments, the polyisobutylene comprises an average of 9 to 28 isobutylene monomeric units per molecule. In certain embodiments, the polyisobutylene comprises an average of 9 to 26 isobutylene monomeric units per molecule. In certain embodiments, the polyisobutylene comprises an average of 9 to 24 isobutylene monomeric units per molecule. In certain embodiments, the polyisobutylene comprises an average of 9 to 22 isobutylene monomeric units per molecule. In certain embodiments, the polyisobutylene comprises an average of 9 to 20 isobutylene monomeric units per molecule. In certain embodiments, the polyisobutylene comprises an average of 9 to 18 isobutylene monomeric units per molecule. In certain embodiments, the polyisobutylene comprises an average of 9 to 16 isobutylene monomeric units per molecule. In certain embodiments, the polyisobutylene comprises an average of 9 to 14 isobutylene monomeric units per molecule. In certain embodiments, the polyisobutylene comprises an average of 9 to 12 isobutylene monomeric units per molecule. In certain embodiments, the polyisobutylene comprises an average of 9 to 10 isobutylene monomeric units per molecule. In certain embodiments, the polyisobutylene comprises an average of 10 to 56 isobutylene monomeric units per molecule. In certain embodiments, the polyisobutylene comprises an average of 10 to 55 isobutylene monomeric units per molecule. In certain embodiments, the polyisobutylene comprises an average of 10 to 54 isobutylene monomeric units per molecule. In certain embodiments, the polyisobutylene comprises an average of 10 to 53 isobutylene monomeric units per molecule. In certain embodiments, the polyisobutylene comprises an average of 10 to 52 isobutylene monomeric units per molecule. In certain embodiments, the polyisobutylene comprises an average of 10 to 51 isobutylene monomeric units per molecule. In certain embodiments, the polyisobutylene comprises an average of 10 to 50 isobutylene monomeric units per molecule. In certain embodiments, the polyisobutylene comprises an average of 10 to 48 isobutylene monomeric units per molecule. In certain embodiments, the polyisobutylene comprises an average of 10 to 46 isobutylene monomeric units per molecule. In certain embodiments, the polyisobutylene comprises an average of 10 to 44 isobutylene monomeric units per molecule. In certain embodiments, the polyisobutylene comprises an average of 10 to 42 isobutylene monomeric units per molecule. In certain embodiments, the polyisobutylene comprises an average of 10 to 40 isobutylene monomeric units per molecule. In certain embodiments, the polyisobutylene comprises an average of 10 to 38 isobutylene monomeric units per molecule. In certain embodiments, the polyisobutylene comprises an average of 10 to 36 isobutylene monomeric units per molecule. In certain embodiments, the polyisobutylene comprises an average of 10 to 34 isobutylene monomeric units per molecule. In certain embodiments, the polyisobutylene comprises an average of 10 to 32 isobutylene monomeric units per molecule. In certain embodiments, the polyisobutylene comprises an average of 10 to 30 isobutylene monomeric units per molecule. In certain embodiments, the polyisobutylene comprises an average of 10 to 28 isobutylene monomeric units per molecule. In certain embodiments, the polyisobutylene comprises an average of 10 to 26 isobutylene monomeric units per molecule. In certain embodiments, the polyisobutylene comprises an average of 10 to 24 isobutylene monomeric units per molecule. In certain embodiments, the polyisobutylene comprises an average of 10 to 22 isobutylene monomeric units per molecule. In certain embodiments, the polyisobutylene comprises an average of 10 to 20 isobutylene monomeric units per molecule. In certain embodiments, the polyisobutylene comprises an average of 10 to 18 isobutylene monomeric units per molecule. In certain embodiments, the polyisobutylene comprises an average of 10 to 16 isobutylene monomeric units per molecule. In certain embodiments, the polyisobutylene comprises an average of 10 to 14 isobutylene monomeric units per molecule. In certain embodiments, the polyisobutylene comprises an average of 10 to 12 isobutylene monomeric units per molecule. In certain embodiments, the polyisobutylene comprises an average of 12 to 56 isobutylene monomeric units per molecule. In certain embodiments, the polyisobutylene comprises an average of 12 to 55 isobutylene monomeric units per molecule. In certain embodiments, the polyisobutylene comprises an average of 12 to 54 isobutylene monomeric units per molecule. In certain embodiments, the polyisobutylene comprises an average of 12 to 53 isobutylene monomeric units per molecule. In certain embodiments, the polyisobutylene comprises an average of 12 to 52 isobutylene monomeric units per molecule. In certain embodiments, the polyisobutylene comprises an average of 12 to 51 isobutylene monomeric units per molecule. In certain embodiments, the polyisobutylene comprises an average of 12 to 50 isobutylene monomeric units per molecule. In certain embodiments, the polyisobutylene comprises an average of 12 to 48 isobutylene monomeric units per molecule. In certain embodiments, the polyisobutylene comprises an average of 12 to 46 isobutylene monomeric units per molecule. In certain embodiments, the polyisobutylene comprises an average of 12 to 44 isobutylene monomeric units per molecule. In certain embodiments, the polyisobutylene comprises an average of 12 to 42 isobutylene monomeric units per molecule. In certain embodiments, the polyisobutylene comprises an average of 12 to 40 isobutylene monomeric units per molecule. In certain embodiments, the polyisobutylene comprises an average of 12 to 38 isobutylene monomeric units per molecule. In certain embodiments, the polyisobutylene comprises an average of 12 to 36 isobutylene monomeric units per molecule. In certain embodiments, the polyisobutylene comprises an average of 12 to 34 isobutylene monomeric units per molecule. In certain embodiments, the polyisobutylene comprises an average of 12 to 32 isobutylene monomeric units per molecule. In certain embodiments, the polyisobutylene comprises an average of 12 to 30 isobutylene monomeric units per molecule. In certain embodiments, the polyisobutylene comprises an average of 12 to 28 isobutylene monomeric units per molecule. In certain embodiments, the polyisobutylene comprises an average of 12 to 26 isobutylene monomeric units per molecule. In certain embodiments, the polyisobutylene comprises an average of 12 to 24 isobutylene monomeric units per molecule. In certain embodiments, the polyisobutylene comprises an average of 12 to 22 isobutylene monomeric units per molecule. In certain embodiments, the polyisobutylene comprises an average of 12 to 20 isobutylene monomeric units per molecule. In certain embodiments, the polyisobutylene comprises an average of 12 to 18 isobutylene monomeric units per molecule. In certain embodiments, the polyisobutylene comprises an average of 12 to 16 isobutylene monomeric units per molecule. In certain embodiments, the polyisobutylene comprises an average of 12 to 14 isobutylene monomeric units per molecule. In certain embodiments, the polyisobutylene comprises an average of 14 to 56 isobutylene monomeric units per molecule. In certain embodiments, the polyisobutylene comprises an average of 14 to 55 isobutylene monomeric units per molecule. In certain embodiments, the polyisobutylene comprises an average of 14 to 54 isobutylene monomeric units per molecule. In certain embodiments, the polyisobutylene comprises an average of 14 to 53 isobutylene monomeric units per molecule. In certain embodiments, the polyisobutylene comprises an average of 14 to 52 isobutylene monomeric units per molecule. In certain embodiments, the polyisobutylene comprises an average of 14 to 51 isobutylene monomeric units per molecule. In certain embodiments, the polyisobutylene comprises an average of 14 to 50 isobutylene monomeric units per molecule. In certain embodiments, the polyisobutylene comprises an average of 14 to 48 isobutylene monomeric units per molecule. In certain embodiments, the polyisobutylene comprises an average of 14 to 46 isobutylene monomeric units per molecule. In certain embodiments, the polyisobutylene comprises an average of 14 to 44 isobutylene monomeric units per molecule. In certain embodiments, the polyisobutylene comprises an average of 14 to 42 isobutylene monomeric units per molecule. In certain embodiments, the polyisobutylene comprises an average of 14 to 40 isobutylene monomeric units per molecule. In certain embodiments, the polyisobutylene comprises an average of 14 to 38 isobutylene monomeric units per molecule. In certain embodiments, the polyisobutylene comprises an average of 14 to 36 isobutylene monomeric units per molecule. In certain embodiments, the polyisobutylene comprises an average of 14 to 34 isobutylene monomeric units per molecule. In certain embodiments, the polyisobutylene comprises an average of 14 to 32 isobutylene monomeric units per molecule. In certain embodiments, the polyisobutylene comprises an average of 14 to 30 isobutylene monomeric units per molecule. In certain embodiments, the polyisobutylene comprises an average of 14 to 28 isobutylene monomeric units per molecule. In certain embodiments, the polyisobutylene comprises an average of 14 to 26 isobutylene monomeric units per molecule. In certain embodiments, the polyisobutylene comprises an average of 14 to 24 isobutylene monomeric units per molecule. In certain embodiments, the polyisobutylene comprises an average of 14 to 22 isobutylene monomeric units per molecule. In certain embodiments, the polyisobutylene comprises an average of 14 to 20 isobutylene monomeric units per molecule. In certain embodiments, the polyisobutylene comprises an average of 14 to 18 isobutylene monomeric units per molecule. In certain embodiments, the polyisobutylene comprises an average of 14 to 16 isobutylene monomeric units per molecule. In certain embodiments, the polyisobutylene comprises an average of 16 to 56 isobutylene monomeric units per molecule. In certain embodiments, the polyisobutylene comprises an average of 16 to 55 isobutylene monomeric units per molecule. In certain embodiments, the polyisobutylene comprises an average of 16 to 54 isobutylene monomeric units per molecule. In certain embodiments, the polyisobutylene comprises an average of 16 to 53 isobutylene monomeric units per molecule. In certain embodiments, the polyisobutylene comprises an average of 16 to 52 isobutylene monomeric units per molecule. In certain embodiments, the polyisobutylene comprises an average of 16 to 51 isobutylene monomeric units per molecule. In certain embodiments, the polyisobutylene comprises an average of 16 to 50 isobutylene monomeric units per molecule. In certain embodiments, the polyisobutylene comprises an average of 16 to 48 isobutylene monomeric units per molecule. In certain embodiments, the polyisobutylene comprises an average of 16 to 46 isobutylene monomeric units per molecule. In certain embodiments, the polyisobutylene comprises an average of 16 to 44 isobutylene monomeric units per molecule. In certain embodiments, the polyisobutylene comprises an average of 16 to 42 isobutylene monomeric units per molecule. In certain embodiments, the polyisobutylene comprises an average of 16 to 40 isobutylene monomeric units per molecule. In certain embodiments, the polyisobutylene comprises an average of 16 to 38 isobutylene monomeric units per molecule. In certain embodiments, the polyisobutylene comprises an average of 16 to 36 isobutylene monomeric units per molecule. In certain embodiments, the polyisobutylene comprises an average of 16 to 34 isobutylene monomeric units per molecule. In certain embodiments, the polyisobutylene comprises an average of 16 to 32 isobutylene monomeric units per molecule. In certain embodiments, the polyisobutylene comprises an average of 16 to 30 isobutylene monomeric units per molecule. In certain embodiments, the polyisobutylene comprises an average of 16 to 28 isobutylene monomeric units per molecule. In certain embodiments, the polyisobutylene comprises an average of 16 to 26 isobutylene monomeric units per molecule. In certain embodiments, the polyisobutylene comprises an average of 16 to 24 isobutylene monomeric units per molecule. In certain embodiments, the polyisobutylene comprises an average of 16 to 22 isobutylene monomeric units per molecule. In certain embodiments, the polyisobutylene comprises an average of 16 to 20 isobutylene monomeric units per molecule. In certain embodiments, the polyisobutylene comprises an average of 16 to 18 isobutylene monomeric units per molecule. In certain embodiments, the polyisobutylene comprises an average of 18 to 56 isobutylene monomeric units per molecule. In certain embodiments, the polyisobutylene comprises an average of 18 to 55 isobutylene monomeric units per molecule. In certain embodiments, the polyisobutylene comprises an average of 18 to 54 isobutylene monomeric units per molecule. In certain embodiments, the polyisobutylene comprises an average of 18 to 53 isobutylene monomeric units per molecule. In certain embodiments, the polyisobutylene comprises an average of 18 to 52 isobutylene monomeric units per molecule. In certain embodiments, the polyisobutylene comprises an average of 18 to 51 isobutylene monomeric units per molecule. In certain embodiments, the polyisobutylene comprises an average of 18 to 50 isobutylene monomeric units per molecule. In certain embodiments, the polyisobutylene comprises an average of 18 to 48 isobutylene monomeric units per molecule. In certain embodiments, the polyisobutylene comprises an average of 18 to 46 isobutylene monomeric units per molecule. In certain embodiments, the polyisobutylene comprises an average of 18 to 44 isobutylene monomeric units per molecule. In certain embodiments, the polyisobutylene comprises an average of 18 to 42 isobutylene monomeric units per molecule. In certain embodiments, the polyisobutylene comprises an average of 18 to 40 isobutylene monomeric units per molecule. In certain embodiments, the polyisobutylene comprises an average of 18 to 38 isobutylene monomeric units per molecule. In certain embodiments, the polyisobutylene comprises an average of 18 to 36 isobutylene monomeric units per molecule. In certain embodiments, the polyisobutylene comprises an average of 18 to 34 isobutylene monomeric units per molecule. In certain embodiments, the polyisobutylene comprises an average of 18 to 32 isobutylene monomeric units per molecule. In certain embodiments, the polyisobutylene comprises an average of 18 to 30 isobutylene monomeric units per molecule. In certain embodiments, the polyisobutylene comprises an average of 18 to 28 isobutylene monomeric units per molecule. In certain embodiments, the polyisobutylene comprises an average of 18 to 26 isobutylene monomeric units per molecule. In certain embodiments, the polyisobutylene comprises an average of 18 to 24 isobutylene monomeric units per molecule. In certain embodiments, the polyisobutylene comprises an average of 18 to 22 isobutylene monomeric units per molecule. In certain embodiments, the polyisobutylene comprises an average of 18 to 20 isobutylene monomeric units per molecule. In certain embodiments, the polyisobutylene comprises an average of 20 to 56 isobutylene monomeric units per molecule. In certain embodiments, the polyisobutylene comprises an average of 20 to 55 isobutylene monomeric units per molecule. In certain embodiments, the polyisobutylene comprises an average of 20 to 54 isobutylene monomeric units per molecule. In certain embodiments, the polyisobutylene comprises an average of 20 to 53 isobutylene monomeric units per molecule. In certain embodiments, the polyisobutylene comprises an average of 20 to 52 isobutylene monomeric units per molecule. In certain embodiments, the polyisobutylene comprises an average of 20 to 51 isobutylene monomeric units per molecule. In certain embodiments, the polyisobutylene comprises an average of 20 to 50 isobutylene monomeric units per molecule. In certain embodiments, the polyisobutylene comprises an average of 20 to 48 isobutylene monomeric units per molecule. In certain embodiments, the polyisobutylene comprises an average of 20 to 46 isobutylene monomeric units per molecule. In certain embodiments, the polyisobutylene comprises an average of 20 to 44 isobutylene monomeric units per molecule. In certain embodiments, the polyisobutylene comprises an average of 20 to 42 isobutylene monomeric units per molecule. In certain embodiments, the polyisobutylene comprises an average of 20 to 40 isobutylene monomeric units per molecule. In certain embodiments, the polyisobutylene comprises an average of 20 to 38 isobutylene monomeric units per molecule. In certain embodiments, the polyisobutylene comprises an average of 20 to 36 isobutylene monomeric units per molecule. In certain embodiments, the polyisobutylene comprises an average of 20 to 34 isobutylene monomeric units per molecule. In certain embodiments, the polyisobutylene comprises an average of 20 to 32 isobutylene monomeric units per molecule. In certain embodiments, the polyisobutylene comprises an average of 20 to 30 isobutylene monomeric units per molecule. In certain embodiments, the polyisobutylene comprises an average of 20 to 28 isobutylene monomeric units per molecule. In certain embodiments, the polyisobutylene comprises an average of 20 to 26 isobutylene monomeric units per molecule. In certain embodiments, the polyisobutylene comprises an average of 20 to 24 isobutylene monomeric units per molecule. In certain embodiments, the polyisobutylene comprises an average of 20 to 22 isobutylene monomeric units per molecule. In certain embodiments, the polyisobutylene comprises an average of 22 to 56 isobutylene monomeric units per molecule. In certain embodiments, the polyisobutylene comprises an average of 22 to 55 isobutylene monomeric units per molecule. In certain embodiments, the polyisobutylene comprises an average of 22 to 54 isobutylene monomeric units per molecule. In certain embodiments, the polyisobutylene comprises an average of 22 to 53 isobutylene monomeric units per molecule. In certain embodiments, the polyisobutylene comprises an average of 22 to 52 isobutylene monomeric units per molecule. In certain embodiments, the polyisobutylene comprises an average of 22 to 51 isobutylene monomeric units per molecule. In certain embodiments, the polyisobutylene comprises an average of 22 to 50 isobutylene monomeric units per molecule. In certain embodiments, the polyisobutylene comprises an average of 22 to 48 isobutylene monomeric units per molecule. In certain embodiments, the polyisobutylene comprises an average of 22 to 46 isobutylene monomeric units per molecule. In certain embodiments, the polyisobutylene comprises an average of 22 to 44 isobutylene monomeric units per molecule. In certain embodiments, the polyisobutylene comprises an average of 22 to 42 isobutylene monomeric units per molecule. In certain embodiments, the polyisobutylene comprises an average of 22 to 40 isobutylene monomeric units per molecule. In certain embodiments, the polyisobutylene comprises an average of 22 to 38 isobutylene monomeric units per molecule. In certain embodiments, the polyisobutylene comprises an average of 22 to 36 isobutylene monomeric units per molecule. In certain embodiments, the polyisobutylene comprises an average of 22 to 34 isobutylene monomeric units per molecule. In certain embodiments, the polyisobutylene comprises an average of 22 to 32 isobutylene monomeric units per molecule. In certain embodiments, the polyisobutylene comprises an average of 22 to 30 isobutylene monomeric units per molecule. In certain embodiments, the polyisobutylene comprises an average of 22 to 28 isobutylene monomeric units per molecule. In certain embodiments, the polyisobutylene comprises an average of 22 to 26 isobutylene monomeric units per molecule. In certain embodiments, the polyisobutylene comprises an average of 22 to 24 isobutylene monomeric units per molecule. In certain embodiments, the polyisobutylene comprises an average of 24 to 56 isobutylene monomeric units per molecule. In certain embodiments, the polyisobutylene comprises an average of 24 to 55 isobutylene monomeric units per molecule. In certain embodiments, the polyisobutylene comprises an average of 24 to 54 isobutylene monomeric units per molecule. In certain embodiments, the polyisobutylene comprises an average of 24 to 53 isobutylene monomeric units per molecule. In certain embodiments, the polyisobutylene comprises an average of 24 to 52 isobutylene monomeric units per molecule. In certain embodiments, the polyisobutylene comprises an average of 24 to 51 isobutylene monomeric units per molecule. In certain embodiments, the polyisobutylene comprises an average of 24 to 50 isobutylene monomeric units per molecule. In certain embodiments, the polyisobutylene comprises an average of 24 to 48 isobutylene monomeric units per molecule. In certain embodiments, the polyisobutylene comprises an average of 24 to 46 isobutylene monomeric units per molecule. In certain embodiments, the polyisobutylene comprises an average of 24 to 44 isobutylene monomeric units per molecule. In certain embodiments, the polyisobutylene comprises an average of 24 to 42 isobutylene monomeric units per molecule. In certain embodiments, the polyisobutylene comprises an average of 24 to 40 isobutylene monomeric units per molecule. In certain embodiments, the polyisobutylene comprises an average of 24 to 38 isobutylene monomeric units per molecule. In certain embodiments, the polyisobutylene comprises an average of 24 to 36 isobutylene monomeric units per molecule. In certain embodiments, the polyisobutylene comprises an average of 24 to 34 isobutylene monomeric units per molecule. In certain embodiments, the polyisobutylene comprises an average of 24 to 32 isobutylene monomeric units per molecule. In certain embodiments, the polyisobutylene comprises an average of 24 to 30 isobutylene monomeric units per molecule. In certain embodiments, the polyisobutylene comprises an average of 24 to 28 isobutylene monomeric units per molecule. In certain embodiments, the polyisobutylene comprises an average of 24 to 26 isobutylene monomeric units per molecule. In certain embodiments, the polyisobutylene comprises an average of 26 to 56 isobutylene monomeric units per molecule. In certain embodiments, the polyisobutylene comprises an average of 26 to 55 isobutylene monomeric units per molecule. In certain embodiments, the polyisobutylene comprises an average of 26 to 54 isobutylene monomeric units per molecule. In certain embodiments, the polyisobutylene comprises an average of 26 to 53 isobutylene monomeric units per molecule. In certain embodiments, the polyisobutylene comprises an average of 26 to 52 isobutylene monomeric units per molecule. In certain embodiments, the polyisobutylene comprises an average of 26 to 51 isobutylene monomeric units per molecule. In certain embodiments, the polyisobutylene comprises an average of 26 to 50 isobutylene monomeric units per molecule. In certain embodiments, the polyisobutylene comprises an average of 26 to 48 isobutylene monomeric units per molecule. In certain embodiments, the polyisobutylene comprises an average of 26 to 46 isobutylene monomeric units per molecule. In certain embodiments, the polyisobutylene comprises an average of 26 to 44 isobutylene monomeric units per molecule. In certain embodiments, the polyisobutylene comprises an average of 26 to 42 isobutylene monomeric units per molecule. In certain embodiments, the polyisobutylene comprises an average of 26 to 40 isobutylene monomeric units per molecule. In certain embodiments, the polyisobutylene comprises an average of 26 to 38 isobutylene monomeric units per molecule. In certain embodiments, the polyisobutylene comprises an average of 26 to 36 isobutylene monomeric units per molecule. In certain embodiments, the polyisobutylene comprises an average of 26 to 34 isobutylene monomeric units per molecule. In certain embodiments, the polyisobutylene comprises an average of 26 to 32 isobutylene monomeric units per molecule. In certain embodiments, the polyisobutylene comprises an average of 26 to 30 isobutylene monomeric units per molecule. In certain embodiments, the polyisobutylene comprises an average of 26 to 28 isobutylene monomeric units per molecule. In certain embodiments, the polyisobutylene comprises an average of 28 to 56 isobutylene monomeric units per molecule. In certain embodiments, the polyisobutylene comprises an average of 28 to 55 isobutylene monomeric units per molecule. In certain embodiments, the polyisobutylene comprises an average of 28 to 54 isobutylene monomeric units per molecule. In certain embodiments, the polyisobutylene comprises an average of 28 to 53 isobutylene monomeric units per molecule. In certain embodiments, the polyisobutylene comprises an average of 28 to 52 isobutylene monomeric units per molecule. In certain embodiments, the polyisobutylene comprises an average of 28 to 51 isobutylene monomeric units per molecule. In certain embodiments, the polyisobutylene comprises an average of 28 to 50 isobutylene monomeric units per molecule. In certain embodiments, the polyisobutylene comprises an average of 28 to 48 isobutylene monomeric units per molecule. In certain embodiments, the polyisobutylene comprises an average of 28 to 46 isobutylene monomeric units per molecule. In certain embodiments, the polyisobutylene comprises an average of 28 to 44 isobutylene monomeric units per molecule. In certain embodiments, the polyisobutylene comprises an average of 28 to 42 isobutylene monomeric units per molecule. In certain embodiments, the polyisobutylene comprises an average of 28 to 40 isobutylene monomeric units per molecule. In certain embodiments, the polyisobutylene comprises an average of 28 to 38 isobutylene monomeric units per molecule. In certain embodiments, the polyisobutylene comprises an average of 28 to 36 isobutylene monomeric units per molecule. In certain embodiments, the polyisobutylene comprises an average of 28 to 34 isobutylene monomeric units per molecule. In certain embodiments, the polyisobutylene comprises an average of 28 to 32 isobutylene monomeric units per molecule. In certain embodiments, the polyisobutylene comprises an average of 28 to 30 isobutylene monomeric units per molecule. In certain embodiments, the polyisobutylene comprises an average of 30 to 56 isobutylene monomeric units per molecule. In certain embodiments, the polyisobutylene comprises an average of 30 to 55 isobutylene monomeric units per molecule. In certain embodiments, the polyisobutylene comprises an average of 30 to 54 isobutylene monomeric units per molecule. In certain embodiments, the polyisobutylene comprises an average of 30 to 53 isobutylene monomeric units per molecule. In certain embodiments, the polyisobutylene comprises an average of 30 to 52 isobutylene monomeric units per molecule. In certain embodiments, the polyisobutylene comprises an average of 30 to 51 isobutylene monomeric units per molecule. In certain embodiments, the polyisobutylene comprises an average of 30 to 50 isobutylene monomeric units per molecule. In certain embodiments, the polyisobutylene comprises an average of 30 to 48 isobutylene monomeric units per molecule. In certain embodiments, the polyisobutylene comprises an average of 30 to 46 isobutylene monomeric units per molecule. In certain embodiments, the polyisobutylene comprises an average of 30 to 44 isobutylene monomeric units per molecule. In certain embodiments, the polyisobutylene comprises an average of 30 to 42 isobutylene monomeric units per molecule. In certain embodiments, the polyisobutylene comprises an average of 30 to 40 isobutylene monomeric units per molecule. In certain embodiments, the polyisobutylene comprises an average of 30 to 38 isobutylene monomeric units per molecule. In certain embodiments, the polyisobutylene comprises an average of 30 to 36 isobutylene monomeric units per molecule. In certain embodiments, the polyisobutylene comprises an average of 30 to 34 isobutylene monomeric units per molecule. In certain embodiments, the polyisobutylene comprises an average of 30 to 32 isobutylene monomeric units per molecule. In certain embodiments, the polyisobutylene comprises an average of 32 to 56 isobutylene monomeric units per molecule. In certain embodiments, the polyisobutylene comprises an average of 32 to 55 isobutylene monomeric units per molecule. In certain embodiments, the polyisobutylene comprises an average of 32 to 54 isobutylene monomeric units per molecule. In certain embodiments, the polyisobutylene comprises an average of 32 to 53 isobutylene monomeric units per molecule. In certain embodiments, the polyisobutylene comprises an average of 32 to 52 isobutylene monomeric units per molecule. In certain embodiments, the polyisobutylene comprises an average of 32 to 51 isobutylene monomeric units per molecule. In certain embodiments, the polyisobutylene comprises an average of 32 to 50 isobutylene monomeric units per molecule. In certain embodiments, the polyisobutylene comprises an average of 32 to 48 isobutylene monomeric units per molecule. In certain embodiments, the polyisobutylene comprises an average of 32 to 46 isobutylene monomeric units per molecule. In certain embodiments, the polyisobutylene comprises an average of 32 to 44 isobutylene monomeric units per molecule. In certain embodiments, the polyisobutylene comprises an average of 32 to 42 isobutylene monomeric units per molecule. In certain embodiments, the polyisobutylene comprises an average of 32 to 40 isobutylene monomeric units per molecule. In certain embodiments, the polyisobutylene comprises an average of 32 to 38 isobutylene monomeric units per molecule. In certain embodiments, the polyisobutylene comprises an average of 32 to 36 isobutylene monomeric units per molecule. In certain embodiments, the polyisobutylene comprises an average of 32 to 34 isobutylene monomeric units per molecule. In certain embodiments, the polyisobutylene comprises an average of 34 to 56 isobutylene monomeric units per molecule. In certain embodiments, the polyisobutylene comprises an average of 34 to 55 isobutylene monomeric units per molecule. In certain embodiments, the polyisobutylene comprises an average of 34 to 54 isobutylene monomeric units per molecule. In certain embodiments, the polyisobutylene comprises an average of 34 to 53 isobutylene monomeric units per molecule. In certain embodiments, the polyisobutylene comprises an average of 34 to 52 isobutylene monomeric units per molecule. In certain embodiments, the polyisobutylene comprises an average of 34 to 51 isobutylene monomeric units per molecule. In certain embodiments, the polyisobutylene comprises an average of 34 to 50 isobutylene monomeric units per molecule. In certain embodiments, the polyisobutylene comprises an average of 34 to 48 isobutylene monomeric units per molecule. In certain embodiments, the polyisobutylene comprises an average of 34 to 46 isobutylene monomeric units per molecule. In certain embodiments, the polyisobutylene comprises an average of 34 to 44 isobutylene monomeric units per molecule. In certain embodiments, the polyisobutylene comprises an average of 34 to 42 isobutylene monomeric units per molecule. In certain embodiments, the polyisobutylene comprises an average of 34 to 40 isobutylene monomeric units per molecule. In certain embodiments, the polyisobutylene comprises an average of 34 to 38 isobutylene monomeric units per molecule. In certain embodiments, the polyisobutylene comprises an average of 34 to 36 isobutylene monomeric units per molecule. In certain embodiments, the polyisobutylene comprises an average of 36 to 56 isobutylene monomeric units per molecule. In certain embodiments, the polyisobutylene comprises an average of 36 to 55 isobutylene monomeric units per molecule. In certain embodiments, the polyisobutylene comprises an average of 36 to 54 isobutylene monomeric units per molecule. In certain embodiments, the polyisobutylene comprises an average of 36 to 53 isobutylene monomeric units per molecule. In certain embodiments, the polyisobutylene comprises an average of 36 to 52 isobutylene monomeric units per molecule. In certain embodiments, the polyisobutylene comprises an average of 36 to 51 isobutylene monomeric units per molecule. In certain embodiments, the polyisobutylene comprises an average of 36 to 50 isobutylene monomeric units per molecule. In certain embodiments, the polyisobutylene comprises an average of 36 to 48 isobutylene monomeric units per molecule. In certain embodiments, the polyisobutylene comprises an average of 36 to 46 isobutylene monomeric units per molecule. In certain embodiments, the polyisobutylene comprises an average of 36 to 44 isobutylene monomeric units per molecule. In certain embodiments, the polyisobutylene comprises an average of 36 to 42 isobutylene monomeric units per molecule. In certain embodiments, the polyisobutylene comprises an average of 36 to 40 isobutylene monomeric units per molecule. In certain embodiments, the polyisobutylene comprises an average of 36 to 38 isobutylene monomeric units per molecule. In certain embodiments, the polyisobutylene comprises an average of 38 to 56 isobutylene monomeric units per molecule. In certain embodiments, the polyisobutylene comprises an average of 38 to 55 isobutylene monomeric units per molecule. In certain embodiments, the polyisobutylene comprises an average of 38 to 54 isobutylene monomeric units per molecule. In certain embodiments, the polyisobutylene comprises an average of 38 to 53 isobutylene monomeric units per molecule. In certain embodiments, the polyisobutylene comprises an average of 38 to 52 isobutylene monomeric units per molecule. In certain embodiments, the polyisobutylene comprises an average of 38 to 51 isobutylene monomeric units per molecule. In certain embodiments, the polyisobutylene comprises an average of 38 to 50 isobutylene monomeric units per molecule. In certain embodiments, the polyisobutylene comprises an average of 38 to 48 isobutylene monomeric units per molecule. In certain embodiments, the polyisobutylene comprises an average of 38 to 46 isobutylene monomeric units per molecule. In certain embodiments, the polyisobutylene comprises an average of 38 to 44 isobutylene monomeric units per molecule. In certain embodiments, the polyisobutylene comprises an average of 38 to 42 isobutylene monomeric units per molecule. In certain embodiments, the polyisobutylene comprises an average of 38 to 40 isobutylene monomeric units per molecule. In certain embodiments, the polyisobutylene comprises an average of 40 to 56 isobutylene monomeric units per molecule. In certain embodiments, the polyisobutylene comprises an average of 40 to 55 isobutylene monomeric units per molecule. In certain embodiments, the polyisobutylene comprises an average of 40 to 54 isobutylene monomeric units per molecule. In certain embodiments, the polyisobutylene comprises an average of 40 to 53 isobutylene monomeric units per molecule. In certain embodiments, the polyisobutylene comprises an average of 40 to 52 isobutylene monomeric units per molecule. In certain embodiments, the polyisobutylene comprises an average of 40 to 51 isobutylene monomeric units per molecule. In certain embodiments, the polyisobutylene comprises an average of 40 to 50 isobutylene monomeric units per molecule. In certain embodiments, the polyisobutylene comprises an average of 40 to 48 isobutylene monomeric units per molecule. In certain embodiments, the polyisobutylene comprises an average of 40 to 46 isobutylene monomeric units per molecule. In certain embodiments, the polyisobutylene comprises an average of 40 to 44 isobutylene monomeric units per molecule. In certain embodiments, the polyisobutylene comprises an average of 40 to 42 isobutylene monomeric units per molecule. In certain embodiments, the polyisobutylene comprises an average of 42 to 56 isobutylene monomeric units per molecule. In certain embodiments, the polyisobutylene comprises an average of 42 to 55 isobutylene monomeric units per molecule. In certain embodiments, the polyisobutylene comprises an average of 42 to 54 isobutylene monomeric units per molecule. In certain embodiments, the polyisobutylene comprises an average of 42 to 53 isobutylene monomeric units per molecule. In certain embodiments, the polyisobutylene comprises an average of 42 to 52 isobutylene monomeric units per molecule. In certain embodiments, the polyisobutylene comprises an average of 42 to 51 isobutylene monomeric units per molecule. In certain embodiments, the polyisobutylene comprises an average of 42 to 50 isobutylene monomeric units per molecule. In certain embodiments, the polyisobutylene comprises an average of 42 to 48 isobutylene monomeric units per molecule. In certain embodiments, the polyisobutylene comprises an average of 42 to 46 isobutylene monomeric units per molecule. In certain embodiments, the polyisobutylene comprises an average of 42 to 44 isobutylene monomeric units per molecule. In certain embodiments, the polyisobutylene comprises an average of 44 to 56 isobutylene monomeric units per molecule. In certain embodiments, the polyisobutylene comprises an average of 44 to 55 isobutylene monomeric units per molecule. In certain embodiments, the polyisobutylene comprises an average of 44 to 54 isobutylene monomeric units per molecule. In certain embodiments, the polyisobutylene comprises an average of 44 to 53 isobutylene monomeric units per molecule. In certain embodiments, the polyisobutylene comprises an average of 44 to 52 isobutylene monomeric units per molecule. In certain embodiments, the polyisobutylene comprises an average of 44 to 51 isobutylene monomeric units per molecule. In certain embodiments, the polyisobutylene comprises an average of 44 to 50 isobutylene monomeric units per molecule. In certain embodiments, the polyisobutylene comprises an average of 44 to 48 isobutylene monomeric units per molecule. In certain embodiments, the polyisobutylene comprises an average of 44 to 46 isobutylene monomeric units per molecule. In certain embodiments, the polyisobutylene comprises an average of 46 to 56 isobutylene monomeric units per molecule. In certain embodiments, the polyisobutylene comprises an average of 46 to 55 isobutylene monomeric units per molecule. In certain embodiments, the polyisobutylene comprises an average of 46 to 54 isobutylene monomeric units per molecule. In certain embodiments, the polyisobutylene comprises an average of 46 to 53 isobutylene monomeric units per molecule. In certain embodiments, the polyisobutylene comprises an average of 46 to 52 isobutylene monomeric units per molecule. In certain embodiments, the polyisobutylene comprises an average of 46 to 51 isobutylene monomeric units per molecule. In certain embodiments, the polyisobutylene comprises an average of 46 to 50 isobutylene monomeric units per molecule. In certain embodiments, the polyisobutylene comprises an average of 46 to 48 isobutylene monomeric units per molecule. In certain embodiments, the polyisobutylene comprises an average of 48 to 56 isobutylene monomeric units per molecule. In certain embodiments, the polyisobutylene comprises an average of 48 to 55 isobutylene monomeric units per molecule. In certain embodiments, the polyisobutylene comprises an average of 48 to 54 isobutylene monomeric units per molecule. In certain embodiments, the polyisobutylene comprises an average of 48 to 53 isobutylene monomeric units per molecule. In certain embodiments, the polyisobutylene comprises an average of 48 to 52 isobutylene monomeric units per molecule. In certain embodiments, the polyisobutylene comprises an average of 48 to 51 isobutylene monomeric units per molecule. In certain embodiments, the polyisobutylene comprises an average of 48 to 50 isobutylene monomeric units per molecule. In certain embodiments, the polyisobutylene comprises an average of 50 to 56 isobutylene monomeric units per molecule. In certain embodiments, the polyisobutylene comprises an average of 50 to 55 isobutylene monomeric units per molecule. In certain embodiments, the polyisobutylene comprises an average of 50 to 54 isobutylene monomeric units per molecule. In certain embodiments, the polyisobutylene comprises an average of 50 to 53 isobutylene monomeric units per molecule. In certain embodiments, the polyisobutylene comprises an average of 50 to 52 isobutylene monomeric units per molecule. In certain embodiments, the polyisobutylene comprises an average of 50 to 51 isobutylene monomeric units per molecule. In certain embodiments, the polyisobutylene comprises an average of 51 to 56 isobutylene monomeric units per molecule. In certain embodiments, the polyisobutylene comprises an average of 51 to 55 isobutylene monomeric units per molecule. In certain embodiments, the polyisobutylene comprises an average of 51 to 54 isobutylene monomeric units per molecule. In certain embodiments, the polyisobutylene comprises an average of 51 to 53 isobutylene monomeric units per molecule. In certain embodiments, the polyisobutylene comprises an average of 51 to 52 isobutylene monomeric units per molecule. In certain embodiments, the polyisobutylene comprises an average of 52 to 56 isobutylene monomeric units per molecule. In certain embodiments, the polyisobutylene comprises an average of 52 to 55 isobutylene monomeric units per molecule. In certain embodiments, the polyisobutylene comprises an average of 52 to 54 isobutylene monomeric units per molecule. In certain embodiments, the polyisobutylene comprises an average of 52 to 53 isobutylene monomeric units per molecule. In certain embodiments, the polyisobutylene comprises an average of 53 to 56 isobutylene monomeric units per molecule. In certain embodiments, the polyisobutylene comprises an average of 53 to 55 isobutylene monomeric units per molecule. In certain embodiments, the polyisobutylene comprises an average of 53 to 54 isobutylene monomeric units per molecule. In certain embodiments, the polyisobutylene comprises an average of 54 to 56 isobutylene monomeric units per molecule. In certain embodiments, the polyisobutylene comprises an average of 54 to 55 isobutylene monomeric units per molecule. In certain embodiments, the polyisobutylene comprises an average of 55 to 56 isobutylene monomeric units per molecule. In certain embodiments, the polyisobutylene comprises an average of 3, 4, 5, 6, 7, 8, 9, 10, 12, 14, 16, 18, 20, 22, 24, 26, 28, 30, 32, 34, 36, 38, 40, 42, 44, 46, 48, 50, 51, 52, 53, 54, 55, or 56 isobutylene monomeric units per molecule.

In certain embodiments, the radical initiator may be any suitable radical initiator, and may comprise an organic peroxide initiator or a peracid initiator.

In certain embodiments, the organic peroxide initiator comprises diglutaroyl, dilauroyl peroxide, benzoyl peroxide, dicumyl peroxide, di(tert butyl) peroxide, tertbutyl hydroperoxide, 3,4-dimethyl-3,4-diphenyl-hexane, tert-butylperoxy-2-ethylhexanoate, tert-butylperoxy-benzoate, tert-butylperoxy-pivalate, tert-butylperoxy-3,5,5-trimethyl-hexanoate, di(2,4-dichlorobenzoyl) peroxide, di(2-methylbenzoyl) peroxide, or di(4-tert-butylcyclohexyl) peroxydicarbonate. Half lives of these and other radical initiators are widely available in the relevant scientific literature.

In certain embodiments, the ethylenically unsaturated acylating agent comprises at least one monoethylenically unsaturated C₃-C₂₈ monocarboxylic acid or ester thereof, or at least one C₄-C₂₈ dicarboxylic acid, anhydride or ester thereof.

In certain embodiments, the ethylenically unsaturated acylating agent may comprise one or more compounds falling within the following general formula.

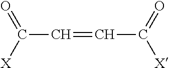

wherein X and X′ are the same or different, provided that at least one of X and X′ is a group that is capable of reacting to esterify alcohols, form amides or amine salts with ammonia or amines, form metal salts with reactive metals or basically reacting metal compounds and otherwise function as acylating agents. In certain embodiments, X and/or X′ is —OH, —O-hydrocarbyl, OM+ where M+ represents one equivalent of a metal, ammonium or amine cation, —NH₂, —Cl, —Br, and/or taken together X and X′ may be —O— so as to form an anhydride. In certain embodiments, X and X′ are such that both carboxylic functions can enter into acylation reactions. Suitable reactants include: maleic anhydride; monophenyl maleic anhydride; monomethyl, dimethyl, monochloro, monobromo, monofluoro, dichloro and difluoro maleic anhydride; N-phenylmaleimide and other substituted maleimides, isomaleimides; fumaric acid, maleic acid, alkyl hydrogen maleates and fumarates, dialkyl fumarates and maleates, fumaronilic acids and maleanic acids; and maleonitrile and fumaronitrile.

In certain embodiments, the monoethylenically unsaturated dicarboxylic acids or anhydrides of 3 to 28 carbon atoms may be selected from the group comprising maleic acid, fumaric acid, itaconic acid, mesaconic acid, methylenemalonic acid, citraconic acid, maleic anhydride, itaconic anhydride, citraconic anhydride and methylenemalonic anhydride and mixtures of these with one another.

In certain embodiments, the monoethylenically unsaturated C₃-C₂₈-monocarboxylic acids may be selected from the group comprising acrylic acid, methacrylic acid, dimethacrylic acid, ethylacrylic acid, crotonic acid, allylacetic acid and vinylacetic acid. Another group of suitable monomers is C₁-C₄₀ alkyl esters of monoethylenecially unsaturated C₃-C₁₀ mono- or dicarboxylic acids such as ethyl acrylate, butyl acrylate, 2-ethyl acrylate, decyl acrylate, docedyl acrylate, loctadecyl acrylate and the esters of industrial alcohol mixtures of 14 to 28 carbon atoms, ethyl methacrylate, 2-ethylhexyl methacrylate, decyl methacrylate, octadecyl methacrylate, monobutyl maleate, dibutyl maleate, monodecyl maleate, didodecyl maleate, monooctadecyl maleate, and dioctadecyl maleate.

In certain embodiments, the ethylenically unsaturated acylating agent comprises maleic acid, fumaric acid, itaconic acid, mesaconic acid, methylenemalonic acid, citraconic acid, maleic anhydride, itaconic anhydride, citraconic anhydride, or methylenemalonic anhydride.

In certain embodiments, the radically-functionalized PIBSA product comprises at least one compound of the following general formula IV:

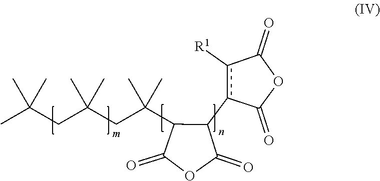

wherein: each R¹ independently represents: —(CH₂)—(C(CH₂))—(CH₃); H; or is not present; wherein when R¹ is not present, the dashed double bond represents a double bond, and when R¹ is present, the dashed double bond represents a single bond; each m independently is an integer from 1 to 52; and each n independently is 0, 1 or 2.

In certain embodiments, each m independently is an integer from 1 to 53, from 1 to 52, from 1 to 51, from 1 to 50, from 1 to 49, from 1 to 48, from 1 to 46, from 1 to 44, from 1 to 42, from 1 to 40, from 1 to 38, from 1 to 36, from 1 to 34, from 1 to 32, from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 7, from 1 to 6, from 1 to 5, from 1 to 4, from 1 to 3, from 1 to 2, from 2 to 54, from 2 to 53, from 2 to 52, from 2 to 51, from 2 to 50, from 2 to 49, from 2 to 48, from 2 to 46, from 2 to 44, from 2 to 42, from 2 to 40, from 2 to 38, from 2 to 36, from 2 to 34, from 2 to 32, from 2 to 30, from 2 to 28, from 2 to 26, from 2 to 24, from 2 to 22, from 2 to 20, from 2 to 18, from 2 to 16, from 2 to 14, from 2 to 12, from 2 to 10, from 2 to 8, from 2 to 7, from 2 to 6, from 2 to 5, from 2 to 4, from 2 to 3, from 3 to 54, from 3 to 53 from 3 to 52, from 3 to 51, from 3 to 50, from 3 to 49, from 3 to 48, from 3 to 46, from 3 to 44, from 3 to 42, from 3 to 40, from 3 to 38, from 3 to 36, from 3 to 34, from 3 to 32, from 3 to 30, from 3 to 28, from 3 to 26, from 3 to 24, from 3 to 22, from 3 to 20, from 3 to 18, from 3 to 16, from 3 to 14, from 3 to 12, from 3 to 10, from 3 to 8, from 3 to 7, from 3 to 6, from 3 to 5, from 3 to 4, from 4 to 54, from 4 to 53, from 4 to 52, from 4 to 51, from 4 to 50, from 4 to 49, from 4 to 48, from 4 to 46, from 4 to 44, from 4 to 42, from 4 to 40, from 4 to 38, from 4 to 36, from 4 to 34, from 4 to 32, from 4 to 30, from 4 to 28, from 4 to 26, from 4 to 24, from 4 to 22, from 4 to 20, from 4 to 18, from 4 to 16, from 4 to 14, from 4 to 12, from 4 to 10, from 4 to 8, from 4 to 7, from 4 to 6, from 4 to 5, from 5 to 54, from 5 to 53, from 5 to 52, from 5 to 51, from 5 to 50, from 5 to 49, from 5 to 48, from 5 to 46, from 5 to 44, from 5 to 42, from 5 to 40, from 5 to 38, from 5 to 36, from 5 to 34, from 5 to 32, from 5 to 30, from 5 to 28, from 5 to 26, from 5 to 24, from 5 to 22, from 5 to 20, from 5 to 18, from 5 to 16, from 5 to 14, from 5 to 12, from 5 to 10, from 5 to 8, from 5 to 7, from 5 to 6, from 6 to 54, from 6 to 53, from 6 to 52, from 6 to 51, from 6 to 50, from 6 to 49, from 6 to 48, from 6 to 46, from 6 to 44, from 6 to 42, from 6 to 40, from 6 to 38, from 6 to 36, from 6 to 34, from 6 to 32, from 6 to 30, from 6 to 28, from 6 to 26, from 6 to 24, from 6 to 22, from 6 to 20, from 6 to 18, from 6 to 16, from 6 to 14, from 6 to 12, from 6 to 10, from 6 to 8, from 6 to 7, from 7 to 54, from 7 to 53, from 7 to 52, from 7 to 51, from 7 to 50, from 7 to 49, from 7 to 48, from 7 to 46, from 7 to 44, from 7 to 42, from 7 to 40, from 7 to 38, from 7 to 36, from 7 to 34, from 7 to 32, from 7 to 30, from 7 to 28, from 7 to 26, from 7 to 24, from 7 to 22, from 7 to 20, from 7 to 18, from 7 to 16, from 7 to 14, from 7 to 12, from 7 to 10, from 7 to 8, from 8 to 54, from 8 to 53, from 8 to 52, from 8 to 51, from 8 to 50, from 8 to 49, from 8 to 48, from 8 to 46, from 8 to 44, from 8 to 42, from 8 to 40, from 8 to 38, from 8 to 36, from 8 to 34, from 8 to 32, from 8 to 30, from 8 to 28, from 8 to 26, from 8 to 24, from 8 to 22, from 8 to 20, from 8 to 18, from 8 to 16, from 8 to 14, from 8 to 12, from 8 to 10, from 10 to 54, from 10 to 53, from 10 to 52, from 10 to 51, from 10 to 50, from 10 to 49, from 10 to 48, from 10 to 46, from 10 to 44, from 10 to 42, from 10 to 40, from 10 to 38, from 10 to 36, from 10 to 34, from 10 to 32, from 10 to 30, from 10 to 28, from 10 to 26, from 10 to 24, from 10 to 22, from 10 to 20, from 10 to 18, from 10 to 16, from 10 to 14, from 10 to 12, from 12 to 54, from 12 to 53, from 12 to 52, from 12 to 51, from 12 to 50, from 12 to 49, from 12 to 48, from 12 to 46, from 12 to 44, from 12 to 42, from 12 to 40, from 12 to 38, from 12 to 36, from 12 to 34, from 12 to 32, from 12 to 30, from 12 to 28, from 12 to 26, from 12 to 24, from 12 to 22, from 12 to 20, from 12 to 18, from 12 to 16, from 12 to 14, from 14 to 54, from 14 to 53, from 14 to 52, from 14 to 51, from 14 to 50, from 14 to 49, from 14 to 48, from 14 to 46, from 14 to 44, from 14 to 42, from 14 to 40, from 14 to 38, from 14 to 36, from 14 to 34, from 14 to 32, from 14 to 30, from 14 to 28, from 14 to 26, from 14 to 24, from 14 to 22, from 14 to 20, from 14 to 18, from 14 to 16, from 16 to 54, from 16 to 53, from 16 to 52, from 16 to 51, from 16 to 50, from 16 to 49, from 16 to 48, from 16 to 46, from 16 to 44, from 16 to 42, from 16 to 40, from 16 to 38, from 16 to 36, from 16 to 34, from 16 to 32, from 16 to 30, from 16 to 28, from 16 to 26, from 16 to 24, from 16 to 22, from 16 to 20, from 16 to 18, from 18 to 54, from 18 to 53, from 18 to 52, from 18 to 51, from 18 to 50, from 18 to 49, from 18 to 48, from 18 to 46, from 18 to 44, from 18 to 42, from 18 to 40, from 18 to 38, from 18 to 36, from 18 to 34, from 18 to 32, from 18 to 30, from 18 to 28, from 18 to 26, from 18 to 24, from 18 to 22, from 18 to 20, from 20 to 54, from 20 to 53, from 20 to 52, from 20 to 51, from 20 to 50, from 20 to 49, from 20 to 48, from 20 to 46, from 20 to 44, from 20 to 42, from 20 to 40, from 20 to 38, from 20 to 36, from 20 to 34, from 20 to 32, from 20 to 30, from 20 to 28, from 20 to 26, from 20 to 24, from 20 to 22, from 22 to 54, from 22 to 53, from 22 to 52, from 22 to 51, from 22 to 50, from 22 to 49, from 22 to 48, from 22 to 46, from 22 to 44, from 22 to 42, from 22 to 40, from 22 to 38, from 22 to 36, from 22 to 34, from 22 to 32, from 22 to 30, from 22 to 28, from 22 to 26, from 22 to 24, from 24 to 54, from 24 to 53, from 24 to 52, from 24 to 51, from 24 to 50, from 24 to 49, from 24 to 48, from 24 to 46, from 24 to 44, from 24 to 42, from 24 to 40, from 24 to 38, from 24 to 36, from 24 to 34, from 24 to 32, from 24 to 30, from 24 to 28, from 24 to 26, from 26 to 54, from 26 to 53, from 26 to 52, from 26 to 51, from 26 to 50, from 26 to 49, from 26 to 48, from 26 to 46, from 26 to 44, from 26 to 42, from 26 to 40, from 26 to 38, from 26 to 36, from 26 to 34, from 26 to 32, from 26 to 30, from 26 to 28, from 28 to 54, from 28 to 53, from 28 to 52, from 28 to 51, from 28 to 50, from 28 to 49, from 28 to 48, from 28 to 46, from 28 to 44, from 28 to 42, from 28 to 40, from 28 to 38, from 28 to 36, from 28 to 34, from 28 to 32, from 28 to 30, from 30 to 54, from 30 to 53, from 30 to 52, from 30 to 51, from 30 to 50, from 30 to 49, from 30 to 48, from 30 to 46, from 30 to 44, from 30 to 42, from 30 to 40, from 30 to 38, from 30 to 36, from 30 to 34, from 30 to 32, from 32 to 54, from 32 to 53, from 32 to 52, from 32 to 51, from 32 to 50, from 32 to 49, from 32 to 48, from 32 to 46, from 32 to 44, from 32 to 42, from 32 to 40, from 32 to 38, from 32 to 36, from 32 to 34, from 34 to 54, from 34 to 53, from 34 to 52, from 34 to 51, from 34 to 50, from 34 to 49, from 34 to 48, from 34 to 46, from 34 to 44, from 34 to 42, from 34 to 40, from 34 to 38, from 34 to 36, from 36 to 54, from 36 to 53, from 36 to 52, from 36 to 51, from 36 to 50, from 36 to 49, from 36 to 48, from 36 to 46, from 36 to 44, from 36 to 42, from 36 to 40, from 36 to 38, from 38 to 54, from 38 to 53, from 38 to 52, from 38 to 51, from 38 to 50, from 38 to 49, from 38 to 48, from 38 to 46, from 38 to 44, from 38 to 42, from 38 to 40, from 40 to 54, from 40 to 53, from 40 to 52, from 40 to 51, from 40 to 50, from 40 to 49, from 40 to 48, from 40 to 46, from 40 to 44, from 40 to 42, from 42 to 54, from 42 to 53, from 42 to 52, from 42 to 51, from 42 to 50, from 42 to 49, from 42 to 48, from 42 to 46, from 42 to 44, from 44 to 54, from 44 to 53, from 44 to 52, from 44 to 51, from 44 to 50, from 44 to 49, from 44 to 48, from 44 to 46, from 46 to 54, from 46 to 53, from 46 to 52, from 46 to 51, from 46 to 50, from 46 to 49, from 46 to 48, from 48 to 54, from 48 to 53, from 48 to 52, from 48 to 51, from 48 to 50, from 48 to 49, from 49 to 54, from 49 to 53, from 49 to 52, from 49 to 51, from 49 to 50, from 50 to 54, from 50 to 53, from 50 to 52, from 50 to 51, from 51 to 54, from 51 to 53, from 51 to 52, from 51 to 51, from 52 to 54, from 52 to 53, or from 53 to 54. In certain embodiments, each m independently is 1, 2, 3, 4, 5, 6, 7, 8, 10, 12, 14, 16, 18, 20, 22, 24, 26, 28, 30, 32, 34, 36, 38, 40, 42, 44, 46, 48, 49, 50, 51, 52, 53, or 54.

In certain embodiments, the radically-functionalized PIBSA product comprises at least 30 (such as at least 35, 40, 45, 50, 55, 60, 65, 70, 75, 80, 85, 90, 95, or 99) mol % of the at least one compound of the general formula I. In certain embodiments, the radically-functionalized PIBSA product comprises from 30 to 100 (such as from 35 to 100, from 40 to 100, from 45 to 100, from 50 to 100, from 55 to 100, from 60 to 100, from 65 to 100, from 70 to 100, from 75 to 100, from 80 to 100, from 85 to 100, from 90 to 100, from 95 to 100, from 99 to 100, from 30 to 99, from 35 to 99, from 40 to 99, from 45 to 99, from 50 to 99, from 55 to 99, from 60 to 99, from 65 to 99, from 70 to 99, from 75 to 99, from 80 to 99, from 85 to 99, from 90 to 99, from 95 to 99, from 30 to 95, from 35 to 95, from 40 to 95, from 45 to 95, from 50 to 95, from 55 to 95, from 60 to 95, from 65 to 95, from 70 to 95, from 75 to 95, from 80 to 95, from 85 to 95, from 90 to 95, from 30 to 90, from 35 to 90, from 40 to 90, from 45 to 90, from 50 to 90, from 55 to 90, from 60 to 90, from 65 to 90, from 70 to 90, from 75 to 90, from 80 to 90, from 85 to 90, from 30 to 85, from 35 to 85, from 40 to 85, from 45 to 85, from 50 to 85, from 55 to 85, from 60 to 85, from 65 to 85, from 70 to 85, from 75 to 85, from 80 to 85, from 30 to 80, from 35 to 80, from 40 to 80, from 45 to 80, from 50 to 80, from 55 to 80, from 60 to 80, from 65 to 80, from 70 to 80, from 75 to 80, from 30 to 75, from 35 to 75, from 40 to 75, from 45 to 75, from 50 to 75, from 55 to 75, from 60 to 75, from 65 to 75, from 70 to 75, from 30 to 70, from 35 to 70, from 40 to 70, from 45 to 70, from 50 to 70, from 55 to 70, from 60 to 70, from 65 to 70, from 30 to 65, from 35 to 65, from 40 to 65, from 45 to 65, from 50 to 65, from 55 to 65, from 60 to 65, from 30 to 60, from 35 to 60, from 40 to 60, from 45 to 60, from 50 to 60, from 55 to 60, from 30 to 55, from 35 to 55, from 40 to 55, from 45 to 55, from 50 to 55, from 30 to 50, from 35 to 50, from 40 to 50, from 45 to 50, from 30 to 45, from 35 to 45, from 40 to 45, from 30 to 40, from 35 to 40 or from 30 to 35) mol % of the at least one compound of the general formula I.

In certain embodiments, the radically-functionalized PIBSA product comprises less than 40 (such as less than 35, 30, 25, 20, 15, 14, 13, 12, 11, 10, 9, 8, 7, 6, 5, 4, 3, 2, or 1) weight percent unreacted polyisobutylene. In certain embodiments, the radically-functionalized PIBSA product comprises from 0 to 40 (such as from 0 to 35, from 0 to 30, from 0 to 25, from 0 to 20, from 0 to 15, from 0 to 14, from 0 to 13, from 0 to 12, from 0 to 11, from 0 to 10, from 0 to 9, from 0 to 8, from 0 to 7, from 0 to 6, from 0 to 5, from 0 to 4, from 0 to 3, from 0 to 2, from 0 to 1, from greater than 0 to 40, from greater than 0 to 35, from greater than 0 to 30, from greater than 0 to 25, from greater than 0 to 20, from greater than 0 to 15, from greater than 0 to 14, from greater than 0 to 13, from greater than 0 to 12, from greater than 0 to 11, from greater than 0 to 10, from greater than 0 to 9, from greater than 0 to 8, from greater than 0 to 7, from greater than 0 to 6, from greater than 0 to 5, from greater than 0 to 4, from greater than 0 to 3, from greater than 0 to 2, from greater than 0 to 1, from greater than 0 to 35, from 1 to 40, from 1 to 35, from 1 to 30, from 1 to 25, from 1 to 20, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, from 2 to 40, from 2 to 35, from 2 to 30, from 2 to 25, from 2 to 20, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, from 2 to 3, from 3 to 40, from 3 to 35, from 3 to 30, from 3 to 25, from 3 to 20, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4, from 4 to 40, from 4 to 35, from 4 to 30, from 4 to 25, from 4 to 20, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 5 to 40, from 5 to 35, from 5 to 30, from 5 to 25, from 5 to 20, from 5 to 15, from 5 to 14, from 5 to 13, from 5 to 12, from 5 to 11, from 5 to 10, from 5 to 9, from 5 to 8, from 5 to 7, from 5 to 6, from 6 to 40, from 6 to 35, from 6 to 30, from 6 to 25, from 6 to 20, from 6 to 15, from 6 to 14, from 6 to 13, from 6 to 12, from 6 to 11, from 6 to 10, from 6 to 9, from 6 to 8, from 6 to 7, from 7 to 40, from 7 to 35, from 7 to 30, from 7 to 25, from 7 to 20, from 7 to 15, from 7 to 14, from 7 to 13, from 7 to 12, from 7 to 11, from 7 to 10, from 7 to 9, from 7 to 8, from 8 to 40, from 8 to 35, from 8 to 30, from 8 to 25, from 8 to 20, from 8 to 15, from 8 to 14, from 8 to 13, from 8 to 12, from 8 to 11, from 8 to 10, from 8 to 9, from 9 to 40, from 9 to 35, from 9 to 30, from 9 to 25, from 9 to 20, from 9 to 15, from 9 to 14, from 9 to 13, from 9 to 12, from 9 to 11, from 9 to 10, from 10 to 40, from 10 to 35, from 10 to 30, from 10 to 25, from 10 to 20, from 10 to 15, from 10 to 14, from 10 to 13, from 10 to 12, from 10 to 11, from 11 to 40, from 11 to 35, from 11 to 30, from 11 to 25, from 11 to 20, from 11 to 15, from 11 to 14, from 11 to 13, from 11 to 12, from 12 to 40, from 12 to 35, from 12 to 30, from 12 to 25, from 12 to 20, from 12 to 15, from 12 to 14, from 12 to 13, from 13 to 40, from 13 to 35, from 13 to 30, from 13 to 25, from 13 to 20, from 13 to 15, from 13 to 14, from 14 to 40, from 14 to 35, from 14 to 30, from 14 to 25, from 14 to 20, from 14 to 15, from 15 to 40, from 15 to 35, from 15 to 30, from 15 to 25, from 15 to 20, from 20 to 40, from 20 to 35, from 20 to 30, from 20 to 25, from 25 to 40, from 25 to 35, from 25 to 30, from 30 to 40, from 30 to 35, or from 35 to 40) weight percent unreacted polyisobutylene.

Also provided are embodiments of the processes to produce the radically-functionalized PIBSA product described above, wherein the process comprises reacting polyisobutylene with a radical initiator and an ethylenically unsaturated acylating agent in two or more additions at a reaction temperature of from 150° C. to 225° C. to produce the radically-functionalized PIBSA product. In certain embodiments, the radical initiator and the ethylenically unsaturated acylating agent are added in two to ten additions (such as two to nine, two to eight, two to seven, two to six, two to five, two to four, two to three, or three additions).

In certain embodiments, each of the two or more additions independently comprises at least 5 (such as at least 10, 15, 20, 25, 30, 35, 40, 45, or 50) weight percent of the total amount of the radical initiator and/or at least 5 (such as at least 10, 15, 20, 25, 30, 35, 40, 45, or 50) weight percent of the total amount of the ethylenically unsaturated acylating agent to be reacted with the polyisobutylene. In certain embodiments, each of the two or more additions independently comprises at most 95 (such as at most 90, 85, 80, 75, 70, 65, 60, 55, 50, 45, 40, 35, 30, 25, 20, 15 or 10) weight percent of the total amount of the radical initiator and/or at most 95 (such as at most 90, 85, 80, 75, 70, 65, 60, 55, 50, 45, 40, 35, 30, 25, 20, 15 or 10) weight percent of the total amount of the ethylenically unsaturated acylating agent to be reacted with the polyisobutylene. In certain embodiments, each of the two or more additions independently comprises from 5 to 95 (such as from 5 to 90, from 5 to 85, from 5 to 80, from 5 to 75, from 5 to 70, from 5 to 65, from 5 to 60, from 5 to 55, from 5 to 50, from 5 to 45, from 5 to 40, from 5 to 35, from 5 to 30, from 5 to 25, from 5 to 20, from 5 to 15, from 5 to 10, from 10 to 95, from 10 to 90, from 10 to 85, from 10 to 80, from 10 to 75, from 10 to 70, from 10 to 65, from 10 to 60, from 10 to 55, from 10 to 50, from 10 to 45, from 10 to 40, from 10 to 35, from 10 to 30, from 10 to 25, from 10 to 20, from 10 to 15, from 15 to 95, from 15 to 90, from 15 to 85, from 15 to 80, from 15 to 75, from 15 to 70, from 15 to 65, from 15 to 60, from 15 to 55, from 15 to 50, from 15 to 45, from 15 to 40, from 15 to 35, from 15 to 30, from 15 to 25, from 15 to 20, from 20 to 95, from 20 to 90, from 20 to 85, from 20 to 80, from 20 to 75, from 20 to 70, from 20 to 65, from 20 to 60, from 20 to 55, from 20 to 50, from 20 to 45, from 20 to 40, from 20 to 35, from 20 to 30, from 20 to 25, from 25 to 95, from 25 to 90, from 25 to 85, from 25 to 80, from 25 to 75, from 25 to 70, from 25 to 65, from 25 to 60, from 25 to 55, from 25 to 50, from 25 to 45, from 25 to 40, from 25 to 35, from 25 to 30, from 30 to 95, from 30 to 90, from 30 to 85, from 30 to 80, from 30 to 75, from 30 to 70, from 30 to 65, from 30 to 60, from 30 to 55, from 30 to 50, from 30 to 45, from 30 to 40, from 30 to 35, from 35 to 95, from 35 to 90, from 35 to 85, from 35 to 80, from 35 to 75, from 35 to 70, from 35 to 65, from 35 to 60, from 35 to 55, from 35 to 50, from 35 to 45, from 35 to 40, from 40 to 95, from 40 to 90, from 40 to 85, from 40 to 80, from 40 to 75, from 40 to 70, from 40 to 65, from 40 to 60, from 40 to 55, from 40 to 50, from 40 to 45, from 45 to 95, from 45 to 90, from 45 to 85, from 45 to 80, from 45 to 75, from 45 to 70, from 45 to 65, from 45 to 60, from 45 to 45, from 45 to 50, from 50 to 95, from 50 to 90, from 50 to 85, from 50 to 80, from 50 to 75, from 50 to 70, from 50 to 65, from 50 to 60, or from 50 to 55) weight percent of the total amount of the radical initiator and/or from 5 to 95 (such as from 5 to 90, from 5 to 85, from 5 to 80, from 5 to 75, from 5 to 70, from 5 to 65, from 5 to 60, from 5 to 55, from 5 to 50, from 5 to 45, from 5 to 40, from 5 to 35, from 5 to 30, from 5 to 25, from 5 to 20, from 5 to 15, from 5 to 10, from 10 to 95, from 10 to 90, from 10 to 85, from 10 to 80, from 10 to 75, from 10 to 70, from 10 to 65, from 10 to 60, from 10 to 55, from 10 to 50, from 10 to 45, from 10 to 40, from 10 to 35, from 10 to 30, from 10 to 25, from 10 to 20, from 10 to 15, from 15 to 95, from 15 to 90, from 15 to 85, from 15 to 80, from 15 to 75, from 15 to 70, from 15 to 65, from 15 to 60, from 15 to 55, from 15 to 50, from 15 to 45, from 15 to 40, from 15 to 35, from 15 to 30, from 15 to 25, from 15 to 20, from 20 to 95, from 20 to 90, from 20 to 85, from 20 to 80, from 20 to 75, from 20 to 70, from 20 to 65, from 20 to 60, from 20 to 55, from 20 to 50, from 20 to 45, from 20 to 40, from 20 to 35, from 20 to 30, from 20 to 25, from 25 to 95, from 25 to 90, from 25 to 85, from 25 to 80, from 25 to 75, from 25 to 70, from 25 to 65, from 25 to 60, from 25 to 55, from 25 to 50, from 25 to 45, from 25 to 40, from 25 to 35, from 25 to 30, from 30 to 95, from 30 to 90, from 30 to 85, from 30 to 80, from 30 to 75, from 30 to 70, from 30 to 65, from 30 to 60, from 30 to 55, from 30 to 50, from 30 to 45, from 30 to 40, from 30 to 35, from 35 to 95, from 35 to 90, from 35 to 85, from 35 to 80, from 35 to 75, from 35 to 70, from 35 to 65, from 35 to 60, from 35 to 55, from 35 to 50, from 35 to 45, from 35 to 40, from 40 to 95, from 40 to 90, from 40 to 85, from 40 to 80, from 40 to 75, from 40 to 70, from 40 to 65, from 40 to 60, from 40 to 55, from 40 to 50, from 40 to 45, from 45 to 95, from 45 to 90, from 45 to 85, from 45 to 80, from 45 to 75, from 45 to 70, from 45 to 65, from 45 to 60, from 45 to 45, from 45 to 50, from 50 to 95, from 50 to 90, from 50 to 85, from 50 to 80, from 50 to 75, from 50 to 70, from 50 to 65, from 50 to 60, or from 50 to 55) weight percent of the total amount of the ethylenically unsaturated acylating agent to be reacted with the polyisobutylene.

In certain embodiments, at least two of the two or more additions are separated in time by at least the half-life of the radical initiator. In certain embodiments, at least two of the two or more additions are separated in time by at least 1.1 (such as at least 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.2, 4.4, 4.6, 4.8, 5, 5.5, 6, 6.5, 7, 7.5, 8, 8.5, 9, 9.5 or 10) times the half-life of the radical initiator. In certain embodiments, each of the two or more additions are separated in times by from 1 to 10 (such as from 1.1 to 10, from 1.2 to 10, from 1.3 to 10, from 1.4 to 10, from 1.5 to 10, from 1.6 to 10, from 1.7 to 10, from 1.8 to 10, from 1.9 to 10, from 2 to 10, from 2.1 to 10, from 2.2 to 10, from 2.3 to 10, from 2.4 to 10, from 2.5 to 10, from 2.6 to 10, from 2.7 to 10, from 2.8 to 10, from 2.9 to 10, from 3 to 10, from 3.1 to 10, from 3.2 to 10, from 3.3 to 10, from 3.4 to 10, from 3.5 to 10, from 3.6 to 10, from 3.7 to 10, from 3.8 to 10, from 3.9 to 10, from 4 to 10, from 4.2 to 10, from 4.4 to 10, from 4.6 to 10, from 4.8 to 10, from 5 to 10, from 5.5 to 10, from 6 to 10, from 6.5 to 10, from 7 to 10, from 7.5 to 10, from 8 to 10, from 8.5 to 10, from 9 to 10, from 9.5 to 10, from 1 to 9.5, from 1.1 to 9.5, from 1.2 to 9.5, from 1.3 to 9.5, from 1.4 to 9.5, from 1.5 to 9.5, from 1.6 to 9.5, from 1.7 to 9.5, from 1.8 to 9.5, from 1.9 to 9.5, from 2 to 9.5, from 2.1 to 9.5, from 2.2 to 9.5, from 2.3 to 9.5, from 2.4 to 9.5, from 2.5 to 9.5, from 2.6 to 9.5, from 2.7 to 9.5, from 2.8 to 9.5, from 2.9 to 9.5, from 3 to 9.5, from 3.1 to 9.5, from 3.2 to 9.5, from 3.3 to 9.5, from 3.4 to 9.5, from 3.5 to 9.5, from 3.6 to 9.5, from 3.7 to 9.5, from 3.8 to 9.5, from 3.9 to 9.5, from 4 to 9.5, from 4.2 to 9.5, from 4.4 to 9.5, from 4.6 to 9.5, from 4.8 to 9.5, from 5 to 9.5, from 5.5 to 9.5, from 6 to 9.5, from 6.5 to 9.5, from 7 to 9.5, from 7.5 to 9.5, from 8 to 9.5, from 8.5 to 9.5, from 9 to 9.5, from 1 to 9, from 1.1 to 9, from 1.2 to 9, from 1.3 to 9, from 1.4 to 9, from 1.5 to 9, from 1.6 to 9, from 1.7 to 9, from 1.8 to 9, from 1.9 to 9, from 2 to 9, from 2.1 to 9, from 2.2 to 9, from 2.3 to 9, from 2.4 to 9, from 2.5 to 9, from 2.6 to 9, from 2.7 to 9, from 2.8 to 9, from 2.9 to 9, from 3 to 9, from 3.1 to 9, from 3.2 to 9, from 3.3 to 9, from 3.4 to 9, from 3.5 to 9, from 3.6 to 9, from 3.7 to 9, from 3.8 to 9, from 3.9 to 9, from 4 to 9, from 4.2 to 9, from 4.4 to 9, from 4.6 to 9, from 4.8 to 9, from 5 to 9, from 5.5 to 9, from 6 to 9, from 6.5 to 9, from 7 to 9, from 7.5 to 9, from 8 to 9, from 8.5 to 9, from 1 to 8.5, from 1.1 to 8.5, from 1.2 to 8.5, from 1.3 to 8.5, from 1.4 to 8.5, from 1.5 to 8.5, from 1.6 to 8.5, from 1.7 to 8.5, from 1.8 to 8.5, from 1.9 to 8.5, from 2 to 8.5, from 2.1 to 8.5, from 2.2 to 8.5, from 2.3 to 8.5, from 2.4 to 8.5, from 2.5 to 8.5, from 2.6 to 8.5, from 2.7 to 8.5, from 2.8 to 8.5, from 2.9 to 8.5, from 3 to 8.5, from 3.1 to 8.5, from 3.2 to 8.5, from 3.3 to 8.5, from 3.4 to 8.5, from 3.5 to 8.5, from 3.6 to 8.5, from 3.7 to 8.5, from 3.8 to 8.5, from 3.9 to 8.5, from 4 to 8.5, from 4.2 to 8.5, from 4.4 to 8.5, from 4.6 to 8.5, from 4.8 to 8.5, from 5 to 8.5, from 5.5 to 8.5, from 6 to 8.5, from 6.5 to 8.5, from 7 to 8.5, from 7.5 to 8.5, from 8 to 8.5, from 1 to 8, from 1.1 to 8, from 1.2 to 8, from 1.3 to 8, from 1.4 to 8, from 1.5 to 8, from 1.6 to 8, from 1.7 to 8, from 1.8 to 8, from 1.9 to 8, from 2 to 8, from 2.1 to 8, from 2.2 to 8, from 2.3 to 8, from 2.4 to 8, from 2.5 to 8, from 2.6 to 8, from 2.7 to 8, from 2.8 to 8, from 2.9 to 8, from 3 to 8, from 3.1 to 8, from 3.2 to 8, from 3.3 to 8, from 3.4 to 8, from 3.5 to 8, from 3.6 to 8, from 3.7 to 8, from 3.8 to 8, from 3.9 to 8, from 4 to 8, from 4.2 to 8, from 4.4 to 8, from 4.6 to 8, from 4.8 to 8, from 5 to 8, from 5.5 to 8, from 6 to 8, from 6.5 to 8, from 7 to 8, from 7.5 to 8, from 1 to 7.5, from 1.1 to 7.5, from 1.2 to 7.5, from 1.3 to 7.5, from 1.4 to 7.5, from 1.5 to 7.5, from 1.6 to 7.5, from 1.7 to 7.5, from 1.8 to 7.5, from 1.9 to 7.5, from 2 to 7.5, from 2.1 to 7.5, from 2.2 to 7.5, from 2.3 to 7.5, from 2.4 to 7.5, from 2.5 to 7.5, from 2.6 to 7.5, from 2.7 to 7.5, from 2.8 to 7.5, from 2.9 to 7.5, from 3 to 7.5, from 3.1 to 7.5, from 3.2 to 7.5, from 3.3 to 7.5, from 3.4 to 7.5, from 3.5 to 7.5, from 3.6 to 7.5, from 3.7 to 7.5, from 3.8 to 7.5, from 3.9 to 7.5, from 4 to 7.5, from 4.2 to 7.5, from 4.4 to 7.5, from 4.6 to 7.5, from 4.8 to 7.5, from 5 to 7.5, from 5.5 to 7.5, from 6 to 7.5, from 6.5 to 7.5, from 7 to 7.5, from 1 to 7, from 1.1 to 7, from 1.2 to 7, from 1.3 to 7, from 1.4 to 7, from 1.5 to 7, from 1.6 to 7, from 1.7 to 7, from 1.8 to 7, from 1.9 to 7, from 2 to 7, from 2.1 to 7, from 2.2 to 7, from 2.3 to 7, from 2.4 to 7, from 2.5 to 7, from 2.6 to 7, from 2.7 to 7, from 2.8 to 7, from 2.9 to 7, from 3 to 7, from 3.1 to 7, from 3.2 to 7, from 3.3 to 7, from 3.4 to 7, from 3.5 to 7, from 3.6 to 7, from 3.7 to 7, from 3.8 to 7, from 3.9 to 7, from 4 to 7, from 4.2 to 7, from 4.4 to 7, from 4.6 to 7, from 4.8 to 7, from 5 to 7, from 5.5 to 7, from 6 to 7, from 6.5 to 7, from 1 to 6.5, from 1.1 to 6.5, from 1.2 to 6.5, from 1.3 to 6.5, from 1.4 to 6.5, from 1.5 to 6.5, from 1.6 to 6.5, from 1.7 to 6.5, from 1.8 to 6.5, from 1.9 to 6.5, from 2 to 6.5, from 2.1 to 6.5, from 2.2 to 6.5, from 2.3 to 6.5, from 2.4 to 6.5, from 2.5 to 6.5, from 2.6 to 6.5, from 2.7 to 6.5, from 2.8 to 6.5, from 2.9 to 6.5, from 3 to 6.5, from 3.1 to 6.5, from 3.2 to 6.5, from 3.3 to 6.5, from 3.4 to 6.5, from 3.5 to 6.5, from 3.6 to 6.5, from 3.7 to 6.5, from 3.8 to 6.5, from 3.9 to 6.5, from 4 to 6.5, from 4.2 to 6.5, from 4.4 to 6.5, from 4.6 to 6.5, from 4.8 to 6.5, from 5 to 6.5, from 5.5 to 6.5, from 6 to 6.5, from 1 to 6, from 1.1 to 6, from 1.2 to 6, from 1.3 to 6, from 1.4 to 6, from 1.5 to 6, from 1.6 to 6, from 1.7 to 6, from 1.8 to 6, from 1.9 to 6, from 2 to 6, from 2.1 to 6, from 2.2 to 6, from 2.3 to 6, from 2.4 to 6, from 2.5 to 6, from 2.6 to 6, from 2.7 to 6, from 2.8 to 6, from 2.9 to 6, from 3 to 6, from 3.1 to 6, from 3.2 to 6, from 3.3 to 6, from 3.4 to 6, from 3.5 to 6, from 3.6 to 6, from 3.7 to 6, from 3.8 to 6, from 3.9 to 6, from 4 to 6, from 4.2 to 6, from 4.4 to 6, from 4.6 to 6, from 4.8 to 6, from 5 to 6, from 5.5 to 6, from 1 to 5.5, from 1.1 to 5.5, from 1.2 to 5.5, from 1.3 to 5.5, from 1.4 to 5.5, from 1.5 to 5.5, from 1.6 to 5.5, from 1.7 to 5.5, from 1.8 to 5.5, from 1.9 to 5.5, from 2 to 5.5, from 2.1 to 5.5, from 2.2 to 5.5, from 2.3 to 5.5, from 2.4 to 5.5, from 2.5 to 5.5, from 2.6 to 5.5, from 2.7 to 5.5, from 2.8 to 5.5, from 2.9 to 5.5, from 3 to 5.5, from 3.1 to 5.5, from 3.2 to 5.5, from 3.3 to 5.5, from 3.4 to 5.5, from 3.5 to 5.5, from 3.6 to 5.5, from 3.7 to 5.5, from 3.8 to 5.5, from 3.9 to 5.5, from 4 to 5.5, from 4.2 to 5.5, from 4.4 to 5.5, from 4.6 to 5.5, from 4.8 to 5.5, from 5 to 5.5, from 1 to 5, from 1.1 to 5, from 1.2 to 5, from 1.3 to 5, from 1.4 to 5, from 1.5 to 5, from 1.6 to 5, from 1.7 to 5, from 1.8 to 5, from 1.9 to 5, from 2 to 5, from 2.1 to 5, from 2.2 to 5, from 2.3 to 5, from 2.4 to 5, from 2.5 to 5, from 2.6 to 5, from 2.7 to 5, from 2.8 to 5, from 2.9 to 5, from 3 to 5, from 3.1 to 5, from 3.2 to 5, from 3.3 to 5, from 3.4 to 5, from 3.5 to 5, from 3.6 to 5, from 3.7 to 5, from 3.8 to 5, from 3.9 to 5, from 4 to 5, from 4.2 to 5, from 4.4 to 5, from 4.6 to 5, from 4.8 to 5, from 1 to 4.8, from 1.1 to 4.8, from 1.2 to 4.8, from 1.3 to 4.8, from 1.4 to 4.8, from 1.5 to 4.8, from 1.6 to 4.8, from 1.7 to 4.8, from 1.8 to 4.8, from 1.9 to 4.8, from 2 to 4.8, from 2.1 to 4.8, from 2.2 to 4.8, from 2.3 to 4.8, from 2.4 to 4.8, from 2.5 to 4.8, from 2.6 to 4.8, from 2.7 to 4.8, from 2.8 to 4.8, from 2.9 to 4.8, from 3 to 4.8, from 3.1 to 4.8, from 3.2 to 4.8, from 3.3 to 4.8, from 3.4 to 4.8, from 3.5 to 4.8, from 3.6 to 4.8, from 3.7 to 4.8, from 3.8 to 4.8, from 3.9 to 4.8, from 4 to 4.8, from 4.2 to 4.8, from 4.4 to 4.8, from 4.6 to 4.8, from 1 to 4.6, from 1.1 to 4.6, from 1.2 to 4.6, from 1.3 to 4.6, from 1.4 to 4.6, from 1.5 to 4.6, from 1.6 to 4.6, from 1.7 to 4.6, from 1.8 to 4.6, from 1.9 to 4.6, from 2 to 4.6, from 2.1 to 4.6, from 2.2 to 4.6, from 2.3 to 4.6, from 2.4 to 4.6, from 2.5 to 4.6, from 2.6 to 4.6, from 2.7 to 4.6, from 2.8 to 4.6, from 2.9 to 4.6, from 3 to 4.6, from 3.1 to 4.6, from 3.2 to 4.6, from 3.3 to 4.6, from 3.4 to 4.6, from 3.5 to 4.6, from 3.6 to 4.6, from 3.7 to 4.6, from 3.8 to 4.6, from 3.9 to 4.6, from 4 to 4.6, from 4.2 to 4.6, from 4.4 to 4.6, from 1 to 4.4, from 1.1 to 4.4, from 1.2 to 4.4, from 1.3 to 4.4, from 1.4 to 4.4, from 1.5 to 4.4, from 1.6 to 4.4, from 1.7 to 4.4, from 1.8 to 4.4, from 1.9 to 4.4, from 2 to 4.4, from 2.1 to 4.4, from 2.2 to 4.4, from 2.3 to 4.4, from 2.4 to 4.4, from 2.5 to 4.4, from 2.6 to 4.4, from 2.7 to 4.4, from 2.8 to 4.4, from 2.9 to 4.4, from 3 to 4.4, from 3.1 to 4.4, from 3.2 to 4.4, from 3.3 to 4.4, from 3.4 to 4.4, from 3.5 to 4.4, from 3.6 to 4.4, from 3.7 to 4.4, from 3.8 to 4.4, from 3.9 to 4.4, from 4 to 4.4, from 4.2 to 4.4, from 1 to 4.2, from 1.1 to 4.2, from 1.2 to 4.2, from 1.3 to 4.2, from 1.4 to 4.2, from 1.5 to 4.2, from 1.6 to 4.2, from 1.7 to 4.2, from 1.8 to 4.2, from 1.9 to 4.2, from 2 to 4.2, from 2.1 to 4.2, from 2.2 to 4.2, from 2.3 to 4.2, from 2.4 to 4.2, from 2.5 to 4.2, from 2.6 to 4.2, from 2.7 to 4.2, from 2.8 to 4.2, from 2.9 to 4.2, from 3 to 4.2, from 3.1 to 4.2, from 3.2 to 4.2, from 3.3 to 4.2, from 3.4 to 4.2, from 3.5 to 4.2, from 3.6 to 4.2, from 3.7 to 4.2, from 3.8 to 4.2, from 3.9 to 4.2, from 4 to 4.2, from 1 to 4, from 1.1 to 4, from 1.2 to 4, from 1.3 to 4, from 1.4 to 4, from 1.5 to 4, from 1.6 to 4, from 1.7 to 4, from 1.8 to 4, from 1.9 to 4, from 2 to 4, from 2.1 to 4, from 2.2 to 4, from 2.3 to 4, from 2.4 to 4, from 2.5 to 4, from 2.6 to 4, from 2.7 to 4, from 2.8 to 4, from 2.9 to 4, from 3 to 4, from 3.1 to 4, from 3.2 to 4, from 3.3 to 4, from 3.4 to 4, from 3.5 to 4, from 3.6 to 4, from 3.7 to 4, from 3.8 to 4, from 3.9 to 4, from 1 to 3.9, from 1.1 to 3.9, from 1.2 to 3.9, from 1.3 to 3.9, from 1.4 to 3.9, from 1.5 to 3.9, from 1.6 to 3.9, from 1.7 to 3.9, from 1.8 to 3.9, from 1.9 to 3.9, from 2 to 3.9, from 2.1 to 3.9, from 2.2 to 3.9, from 2.3 to 3.9, from 2.4 to 3.9, from 2.5 to 3.9, from 2.6 to 3.9, from 2.7 to 3.9, from 2.8 to 3.9, from 2.9 to 3.9, from 3 to 3.9, from 3.1 to 3.9, from 3.2 to 3.9, from 3.3 to 3.9, from 3.4 to 3.9, from 3.5 to 3.9, from 3.6 to 3.9, from 3.7 to 3.9, from 3.8 to 3.9, from 1 to 3.8, from 1.1 to 3.8, from 1.2 to 3.8, from 1.3 to 3.8, from 1.4 to 3.8, from 1.5 to 3.8, from 1.6 to 3.8, from 1.7 to 3.8, from 1.8 to 3.8, from 1.9 to 3.8, from 2 to 3.8, from 2.1 to 3.8, from 2.2 to 3.8, from 2.3 to 3.8, from 2.4 to 3.8, from 2.5 to 3.8, from 2.6 to 3.8, from 2.7 to 3.8, from 2.8 to 3.8, from 2.9 to 3.8, from 3 to 3.8, from 3.1 to 3.8, from 3.2 to 3.8, from 3.3 to 3.8, from 3.4 to 3.8, from 3.5 to 3.8, from 3.6 to 3.8, from 3.7 to 3.8, from 1 to 3.7, from 1.1 to 3.7, from 1.2 to 3.7, from 1.3 to 3.7, from 1.4 to 3.7, from 1.5 to 3.7, from 1.6 to 3.7, from 1.7 to 3.7, from 1.8 to 3.7, from 1.9 to 3.7, from 2 to 3.7, from 2.1 to 3.7, from 2.2 to 3.7, from 2.3 to 3.7, from 2.4 to 3.7, from 2.5 to 3.7, from 2.6 to 3.7, from 2.7 to 3.7, from 2.8 to 3.7, from 2.9 to 3.7, from 3 to 3.7, from 3.1 to 3.7, from 3.2 to 3.7, from 3.3 to 3.7, from 3.4 to 3.7, from 3.5 to 3.7, from 3.6 to 3.7, from 1 to 3.6, from 1.1 to 3.6, from 1.2 to 3.6, from 1.3 to 3.6, from 1.4 to 3.6, from 1.5 to 3.6, from 1.6 to 3.6, from 1.7 to 3.6, from 1.8 to 3.6, from 1.9 to 3.6, from 2 to 3.6, from 2.1 to 3.6, from 2.2 to 3.6, from 2.3 to 3.6, from 2.4 to 3.6, from 2.5 to 3.6, from 2.6 to 3.6, from 2.7 to 3.6, from 2.8 to 3.6, from 2.9 to 3.6, from 3 to 3.6, from 3.1 to 3.6, from 3.2 to 3.6, from 3.3 to 3.6, from 3.4 to 3.6, from 3.5 to 3.6, from 1 to 3.5, from 1.1 to 3.5, from 1.2 to 3.5, from 1.3 to 3.5, from 1.4 to 3.5, from 1.5 to 3.5, from 1.6 to 3.5, from 1.7 to 3.5, from 1.8 to 3.5, from 1.9 to 3.5, from 2 to 3.5, from 2.1 to 3.5, from 2.2 to 3.5, from 2.3 to 3.5, from 2.4 to 3.5, from 2.5 to 3.5, from 2.6 to 3.5, from 2.7 to 3.5, from 2.8 to 3.5, from 2.9 to 3.5, from 3 to 3.5, from 3.1 to 3.5, from 3.2 to 3.5, from 3.3 to 3.5, from 3.4 to 3.5, from 1 to 3.4, from 1.1 to 3.4, from 1.2 to 3.4, from 1.3 to 3.4, from 1.4 to 3.4, from 1.5 to 3.4, from 1.6 to 3.4, from 1.7 to 3.4, from 1.8 to 3.4, from 1.9 to 3.4, from 2 to 3.4, from 2.1 to 3.4, from 2.2 to 3.4, from 2.3 to 3.4, from 2.4 to 3.4, from 2.5 to 3.4, from 2.6 to 3.4, from 2.7 to 3.4, from 2.8 to 3.4, from 2.9 to 3.4, from 3 to 3.4, from 3.1 to 3.4, from 3.2 to 3.4, from 3.3 to 3.4, from 1 to 3.3, from 1.1 to 3.3, from 1.2 to 3.3, from 1.3 to 3.3, from 1.4 to 3.3, from 1.5 to 3.3, from 1.6 to 3.3, from 1.7 to 3.3, from 1.8 to 3.3, from 1.9 to 3.3, from 2 to 3.3, from 2.1 to 3.3, from 2.2 to 3.3, from 2.3 to 3.3, from 2.4 to 3.3, from 2.5 to 3.3, from 2.6 to 3.3, from 2.7 to 3.3, from 2.8 to 3.3, from 2.9 to 3.3, from 3 to 3.3, from 3.1 to 3.3, from 3.2 to 3.3, from 1 to 3.2, from 1.1 to 3.2, from 1.2 to 3.2, from 1.3 to 3.2, from 1.4 to 3.2, from 1.5 to 3.2, from 1.6 to 3.2, from 1.7 to 3.2, from 1.8 to 3.2, from 1.9 to 3.2, from 2 to 3.2, from 2.1 to 3.2, from 2.2 to 3.2, from 2.3 to 3.2, from 2.4 to 3.2, from 2.5 to 3.2, from 2.6 to 3.2, from 2.7 to 3.2, from 2.8 to 3.2, from 2.9 to 3.2, from 3 to 3.2, from 3.1 to 3.2, from 1 to 3.1, from 1.1 to 3.1, from 1.2 to 3.1, from 1.3 to 3.1, from 1.4 to 3.1, from 1.5 to 3.1, from 1.6 to 3.1, from 1.7 to 3.1, from 1.8 to 3.1, from 1.9 to 3.1, from 2 to 3.1, from 2.1 to 3.1, from 2.2 to 3.1, from 2.3 to 3.1, from 2.4 to 3.1, from 2.5 to 3.1, from 2.6 to 3.1, from 2.7 to 3.1, from 2.8 to 3.1, from 2.9 to 3.1, from 3 to 3.1, from 1 to 3, from 1.1 to 3, from 1.2 to 3, from 1.3 to 3, from 1.4 to 3, from 1.5 to 3, from 1.6 to 3, from 1.7 to 3, from 1.8 to 3, from 1.9 to 3, from 2 to 3, from 2.1 to 3, from 2.2 to 3, from 2.3 to 3, from 2.4 to 3, from 2.5 to 3, from 2.6 to 3, from 2.7 to 3, from 2.8 to 3, from 2.9 to 3, from 1 to 2.9, from 1.1 to 2.9, from 1.2 to 2.9, from 1.3 to 2.9, from 1.4 to 2.9, from 1.5 to 2.9, from 1.6 to 2.9, from 1.7 to 2.9, from 1.8 to 2.9, from 1.9 to 2.9, from 2 to 2.9, from 2.1 to 2.9, from 2.2 to 2.9, from 2.3 to 2.9, from 2.4 to 2.9, from 2.5 to 2.9, from 2.6 to 2.9, from 2.7 to 2.9, from 2.8 to 2.9, from 1 to 2.8, from 1.1 to 2.8, from 1.2 to 2.8, from 1.3 to 2.8, from 1.4 to 2.8, from 1.5 to 2.8, from 1.6 to 2.8, from 1.7 to 2.8, from 1.8 to 2.8, from 1.9 to 2.8, from 2 to 2.8, from 2.1 to 2.8, from 2.2 to 2.8, from 2.3 to 2.8, from 2.4 to 2.8, from 2.5 to 2.8, from 2.6 to 2.8, from 2.7 to 2.8, from 1 to 2.7, from 1.1 to 2.7, from 1.2 to 2.7, from 1.3 to 2.7, from 1.4 to 2.7, from 1.5 to 2.7, from 1.6 to 2.7, from 1.7 to 2.7, from 1.8 to 2.7, from 1.9 to 2.7, from 2 to 2.7, from 2.1 to 2.7, from 2.2 to 2.7, from 2.3 to 2.7, from 2.4 to 2.7, from 2.5 to 2.7, from 2.6 to 2.7, from 1 to 2.6, from 1.1 to 2.6, from 1.2 to 2.6, from 1.3 to 2.6, from 1.4 to 2.6, from 1.5 to 2.6, from 1.6 to 2.6, from 1.7 to 2.6, from 1.8 to 2.6, from 1.9 to 2.6, from 2 to 2.6, from 2.1 to 2.6, from 2.2 to 2.6, from 2.3 to 2.6, from 2.4 to 2.6, from 2.5 to 2.6, from 1 to 2.5, from 1.1 to 2.5, from 1.2 to 2.5, from 1.3 to 2.5, from 1.4 to 2.5, from 1.5 to 2.5, from 1.6 to 2.5, from 1.7 to 2.5, from 1.8 to 2.5, from 1.9 to 2.5, from 2 to 2.5, from 2.1 to 2.5, from 2.2 to 2.5, from 2.3 to 2.5, from 2.4 to 2.5, from 1 to 2.4, from 1.1 to 2.4, from 1.2 to 2.4, from 1.3 to 2.4, from 1.4 to 2.4, from 1.5 to 2.4, from 1.6 to 2.4, from 1.7 to 2.4, from 1.8 to 2.4, from 1.9 to 2.4, from 2 to 2.4, from 2.1 to 2.4, from 2.2 to 2.4, from 2.3 to 2.4, from 1 to 2.3, from 1.1 to 2.3, from 1.2 to 2.3, from 1.3 to 2.3, from 1.4 to 2.3, from 1.5 to 2.3, from 1.6 to 2.3, from 1.7 to 2.3, from 1.8 to 2.3, from 1.9 to 2.3, from 2 to 2.3, from 2.1 to 2.3, from 2.2 to 2.3, from 1 to 2.2, from 1.1 to 2.2, from 1.2 to 2.2, from 1.3 to 2.2, from 1.4 to 2.2, from 1.5 to 2.2, from 1.6 to 2.2, from 1.7 to 2.2, from 1.8 to 2.2, from 1.9 to 2.2, from 2 to 2.2, from 2.1 to 2.2, from 1 to 2.1, from 1.1 to 2.1, from 1.2 to 2.1, from 1.3 to 2.1, from 1.4 to 2.1, from 1.5 to 2.1, from 1.6 to 2.1, from 1.7 to 2.1, from 1.8 to 2.1, from 1.9 to 2.1, from 2 to 2.1, from 1 to 2, from 1.1 to 2, from 1.2 to 2, from 1.3 to 2, from 1.4 to 2, from 1.5 to 2, from 1.6 to 2, from 1.7 to 2, from 1.8 to 2, from 1.9 to 2, from 1 to 1.9, from 1.1 to 1.9, from 1.2 to 1.9, from 1.3 to 1.9, from 1.4 to 1.9, from 1.5 to 1.9, from 1.6 to 1.9, from 1.7 to 1.9, from 1.8 to 1.9, from 1 to 1.8, from 1.1 to 1.8, from 1.2 to 1.8, from 1.3 to 1.8, from 1.4 to 1.8, from 1.5 to 1.8, from 1.6 to 1.8, from 1.7 to 1.8, from 1 to 1.7, from 1.1 to 1.7, from 1.2 to 1.7, from 1.3 to 1.7, from 1.4 to 1.7, from 1.5 to 1.7, from 1.6 to 1.7, from 1 to 1.6, from 1.1 to 1.6, from 1.2 to 1.6, from 1.3 to 1.6, from 1.4 to 1.6, from 1.5 to 1.6, from 1 to 1.5, from 1.1 to 1.5, from 1.2 to 1.5, from 1.3 to 1.5, from 1.4 to 1.5, from 1 to 1.4, from 1.1 to 1.4, from 1.2 to 1.4, from 1.3 to 1.4, from 1 to 1.3, from 1.1 to 1.3, from 1.2 to 1.3, from 1 to 1.2, from 1.1 to 1.3, or from 1 to 1.1) times the half-life of the radical initiator.

Also provided are embodiments of the processes to produce the radically-functionalized PIBSA product described above, wherein the process comprises reacting polyisobutylene (such as high-vinylidene polyisobutylene) with a radical initiator and an ethylenically unsaturated acylating agent at a temperature of from 150° C. to 225° C. to produce the radically-functionalized PIBSA product, wherein the polyisobutylene is brought to the reaction temperature in a continuous reactor, prior to commencing continuous addition of the radical initiator to the polyisobutylene in the continuous reactor; and wherein the ethylenically unsaturated acylating agent is added to the continuous reactor: (i) as a mixture with the polyisobutylene; (ii) as a mixture with the radical initiator; and/or (iii) continuously and separately from either the polyisobutylene or the radical initiator.

An illustrative, non-limiting embodiment of the processes to produce the radically-functionalized PIBSA product described herein may include feeding polyisobutylene into a two- or three-stage reactor system at 170° C., with each stage including an addition of maleic anhydride acylating agent and di-tertbutyl peroxide initiator to the polyisobutylene flowing through the reactor system. With the half-life of di-tertbutyl peroxide being about 6 minutes, each stage is allowed to react for about 20 minutes to allow most of the initiator to be consumed before adding another dose. After two or three stages, in about 40 to 60 minutes, the reaction is complete and the desired product has been achieved.

### Nitrogen-Containing Compounds

In certain embodiments, the nitrogen-containing compound has: (a) a nitrogen atom capable of reacting with the radically-functionalized PIBSA product to form an imide; and (b) at least one quaternizable amino group. In certain embodiments, the quaternizable amino group may be a primary, secondary or tertiary amino group. A quaternizable amino group is any primary, secondary, or tertiary amino group on the nitrogen containing compound that is available to react with a quaternizing agent to become a quaternary amino group. In certain embodiments, the quaternizing agent is suitable for converting the quaternizable amino group to a quaternary nitrogen group.

In certain embodiments, the nitrogen-containing compound may be represented by the following formula:

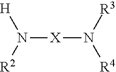

wherein X is an alkylene group containing 1 to 4 carbon atoms; R² is hydrogen or a hydrocarbyl group; and R³ and R⁴ are hydrocarbyl groups.

Suitable, non-limiting examples of the nitrogen-containing compound include: N,N dimethyl-aminopropylamine, N,N-diethyl-aminopropylamine, N,N-dimethyl-aminoethylamine ethylenediamine, 1,2-propylenediamine, 1,3-propylene diamine, isomeric amines, including butylenediamines, pentanediamines, hexanediamines, and heptanediamines, diethylenetriamine, dipropylenetriamine, dibutylenetriamine, triethylenetetramine, tetraethylenepentamine, pentaethylenehexamine, hexamethylenetetramine, and bis(hexamethylene) triamine, the diaminobenzenes, the diaminopyridines, N-methyl-3-amino-1-propylamine, or mixtures thereof. The nitrogen containing compounds capable of reacting with the acylating agent and further having a quaternizable amino group can further include aminoalkyl substituted heterocyclic compounds such as 1-(3-aminopropyl)imidazole and 4-(3-aminopropyl)morpholine, 1 (2-aminoethyl)piperidine, 3,3-diamino-N-methyldipropylamine. In some embodiments, the nitrogen containing compound excludes dimethylaminopropylamine.

In certain embodiments, the nitrogen-containing compound may be an imidazole, for example an imidazole of the following formula:

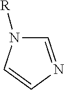

wherein R is an amine capable of reacting in the processes described herein and having from 3 to 8 carbon atoms.

In certain embodiments, the nitrogen-containing compound may be represented by the following formula:

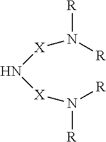

wherein each X is, individually, a C₁ to C₆ hydrocarbylene group, and each R is, individually, a hydrogen or a C₁ to C₆ hydrocarbyl group. In certain embodiments, X may be, individually, a C₁, C₂, or C₃ alkylene group. In certain embodiments, each R may be, individually, H or a C₁, C₂, or C₃ alkyl group.

### Quaternizable Compounds

In certain embodiments, the reaction to prepare the quaternizable compound may be carried out at a temperature of greater than 80 (such as greater than 90 or greater than 100) ° C. In certain embodiments, the reaction to prepare the quaternizable compound may be carried out at a temperature of from 80 to 200 (such as from 90 to 200, from 100 to 200, from 125 to 200, from 80 to 175, from 90 to 175, from 100 to 175, from 125 to 175, from 80 to 150, from 90 to 150, from 100 to 150, or from 125 to 150) ° C. In certain embodiments, the water of reaction, such as water produced during the condensation reaction may be removed.

The radically-functionalized PIBSA product and the nitrogen-containing compound may be reacted at a ratio of from 3:1 to 1:1.2, or from 1.5:1 to 1:1.1, or from 2:1 to 1:1.05, such as at a ratio of 1:1.

### Quaternizing Agent

In certain embodiments, the quaternizing agent comprises at least one of a dialkyl sulfate, an alkyl halide, a hydrocarbyl substituted carbonate, or a hydrocarbyl epoxide. In certain embodiments, the quaternizing agent comprises a hydrocarbyl epoxide in combination with an acid.

In certain embodiments, the quaternizing agent may comprise alkyl halides, such as chlorides, iodides or bromides; alkyl sulfonates; dialkyl sulfates, such as, dimethyl sulfate and diethyl sulfate; sultones; alkyl phosphates; such as, C₁₋₁₂ trialkylphosphates; di C₁₋₁₂ alkylphosphates; borates; C₁-12 alkyl borates; alkyl nitrites; alkyl nitrates; dialkyl carbonates, such as dimethyl oxalate; alkyl alkanoates; aryl alkanoates, such as methylsalicylate; O,O-di-C₁₋₁₂ alkyldithiophosphates; or mixtures thereof.

In certain embodiments, the quaternizing agent may be derived from dialkyl sulfates such as dimethyl sulfate or diethyl sulfate, N-oxides, sultones such as propane and butane sultone; alkyl, acyl or aryl halides such as methyl and ethyl chloride, bromide or iodide or benzyl chloride, and a hydrocarbyl (or alkyl) substituted carbonates. If the alkyl halide is benzyl chloride, the aromatic ring is optionally further substituted with alkyl or alkenyl groups.

In certain embodiments, the hydrocarbyl groups of the hydrocarbyl substituted carbonates may contain 1 to 50, 1 to 20, 1 to 10, or 1 to 5 carbon atoms per group. In one embodiment, the hydrocarbyl substituted carbonates contain two hydrocarbyl groups that may be the same or different. Examples of suitable hydrocarbyl substituted carbonates include dimethyl carbonate or diethyl carbonate.

In certain embodiments, the quaternizing agent may comprise a hydrocarbyl epoxide, for example a hydrocarbyl epoxide as represented by the following formula:

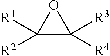

wherein each R¹, R², R³, and R⁴ are, independently, H or a hydrocarbyl group having from 1 to 50 carbon atoms. Examples of hydrocarbyl epoxides include: ethylene oxide, propylene oxide, butylene oxide, styrene oxide, and combinations thereof. In certain embodiments, the hydrocarbyl epoxide can be an alcohol functionalized epoxide, C₄ to C₁₄ epoxides, and mixtures thereof. Illustrative C₄ to C₁₄ epoxides are those where each R¹, R², R³, and R⁴ are, independently, H or a C₂ to C₁₂ hydrocarbyl group. In certain embodiments, the epoxides can be C₄ to C₁₄ epoxides. Epoxides suitable as quaternizing agents in the present technology may include, for example, C₄ to C₁₄ epoxides having linear hydrocarbyl substituents, such as, for example, 2-ethyloxirane, 2-propyloxirane, and the like, and C₄ to C₁₄ epoxides having branched and cyclic or aromatic substituents, such as, for example, styrene oxide. C₄ to C₁₄ epoxides can also include epoxidized tri-glycerides, fats or oils; epoxidized alkyl esters of fatty acids; and mixtures thereof. In certain embodiments, the hydrocarbyl epoxide may be a C₄ to C₂₀ epoxide. Illustrative alcohol functionalized epoxides can include those where each R¹, R², R³, and R⁴ are, independently, H or a hydroxyl containing hydrocarbyl group. In certain embodiments, hydroxyl containing hydrocarbyl group can contain from 2 to 32, or from 3 to 28, or even from 3 to 24, carbon atoms. Illustrative alcohol functionalized epoxide derivatives can include for example, glycidol and the like.

In certain embodiments, the hydrocarbyl epoxide may be employed in combination with an acid. The acid used with the hydrocarbyl epoxide may be a separate component, such as acetic acid. In certain embodiments, a small amount of an acid component may be present, but at <0.2, or <0.1, moles of acid per mole of hydrocarbyl acylating agent. These acids may also be used with the other quaternizing agents described herein. Suitable acids may also include, for example, carboxylic acid (such as acetic acid and/or tall oil fatty acid), propionic acid, 2-ethylhexanoic acid, and the like, and mixtures thereof.

In certain embodiments, the quaternizing agent may comprise an ester of a carboxylic acid capable of reacting with a tertiary amine to form a quaternary ammonium salt, or an ester of a polycarboxylic acid. Such materials may be described, in certain embodiments, as compounds having the structure: R¹⁹—C(═O)—O—R²⁰; wherein R¹⁹ is an (optionally substituted) alkyl, alkenyl, aryl, or alkylaryl group, and R²¹ is a hydrocarbyl group containing from 1 to 22 carbon atoms. Suitable compounds include, without limitation, esters of carboxylic acids having a pKa of 3.5 or less. In certain embodiments, the compound is an ester of a carboxylic acid comprising at least one of a substituted aromatic carboxylic acid, an α-hydroxycarboxylic acid, or a polycarboxylic acid. In certain embodiments, the compound is an ester of a substituted aromatic carboxylic acid, and thus R¹⁹ is a substituted aryl group. R¹⁹ may be a substituted aryl group having 6 to 10 carbon atoms, a phenyl group, or a naphthyl group. R¹⁹ may be suitably substituted with one or more groups selected from carboalkoxy, nitro, cyano, hydroxy, SR′ or NR′R″ where each of R′ and R″ may independently be hydrogen, or an optionally substituted alkyl, alkenyl, aryl or carboalkoxy groups. In certain embodiments, R′ and R″ are each independently hydrogen or an (optionally substituted) alkyl group containing from 1 to 22, 1 to 16, 1 to 10, or 1 to 4, carbon atoms. In certain embodiments, R¹⁹ is an aryl group substituted with one or more groups selected from hydroxyl, carboalkoxy, nitro, cyano and NH₂. R¹⁹ may be a poly-substituted aryl group, for example trihydroxyphenyl, but may also be a mono-substituted aryl group, for example an ortho substituted aryl group. R¹⁹ may be substituted with a group selected from OH, NH₂, NO₂, or COOMe. R¹⁹ may be a hydroxy substituted aryl group. In certain embodiments, R¹⁹ is a 2-hydroxyphenyl group. R²⁰ may be an alkyl or alkylaryl group, for example an alkyl or alkylaryl group containing from 1 to 16 carbon atoms, or from 1 to 10, or 1 to 8 carbon atoms. R²⁰ may be methyl, ethyl, propyl, butyl, pentyl, benzyl or an isomer thereof. In certain embodiments, R²⁰ is benzyl or methyl. In certain embodiments, the quaternizing agent is methyl salicylate.

In certain embodiments, the quaternizing agent may comprise an ester of an alpha-hydroxycarboxylic acid. Examples of suitable compounds which contain the residue of an alpha-hydroxycarboxylic acid include: (i) methyl-, ethyl-, propyl-, butyl-, pentyl-, hexyl-, benzyl-, phenyl-, and allyl esters of 2-hydroxyisobutyric acid; (ii) methyl-, ethyl-, propyl-, butyl-, pentyl-, hexyl-, benzyl-, phenyl-, and allyl esters of 2-hydroxy-2-methylbutyric acid; (iii) methyl-, ethyl-, propyl-, butyl-, pentyl-, hexyl-, benzyl-, phenyl-, and allyl esters of 2-hydroxy-2-ethylbutyric acid; (iv) methyl-, ethyl-, propyl-, butyl-, pentyl-, hexyl-, benzyl-, phenyl-, and allyl esters of lactic acid; and (v) methyl-, ethyl-, propyl-, butyl-, pentyl-, hexyl-, allyl-, benzyl-, and phenyl esters of glycolic acid. In certain embodiments the quaternizing agent comprises methyl 2-hydroxyisobutyrate.

In certain embodiments, the quaternizing agent comprises an ester of a polycarboxylic acid. Included in this definition are dicarboxylic acids and carboxylic acids having more than 2 acidic moieties. In certain embodiments, the esters are alkyl esters with alkyl groups that contain from 1 to 4 carbon atoms. Suitable examples include diesters of oxalic acid, diesters of phthalic acid, diesters of maleic acid, diesters of malonic acid, or diesters or triesters of citric acid.

In certain embodiments, the quaternizing agent is an ester of a carboxylic acid having a pKa of less than 3.5. In certain embodiments, in which the compound includes more than one acid group, reference is made to the first dissociation constant. The quaternizing agent may be selected from an ester of a carboxylic acid selected from one or more of oxalic acid, phthalic acid, salicylic acid, maleic acid, malonic acid, citric acid, nitrobenzoic acid, aminobenzoic acid, and 2, 4, 6-trihydroxybenzoic acid. In certain embodiments, the quaternizing agent may include dimethyl oxalate, a terephthalate (such as dimethyl terephthalate), and methyl 2-nitrobenzoate.

Quaternizing agents capable of coupling more than one quaternizable compound also may be employed, for example. By “coupling” more than one quaternizable compound, it is meant that at least two quaternizable compounds react with the same quaternizing agent to form a compound of the at least two quaternizable compounds linked by the quaternizing agent. Such quaternizing agents may, in some instances, also be referred to as coupling quaternizing agents herein and can include, for example, polyepoxides, such as, for example, di-, tri-, or higher epoxides; polyhalides; epoxy-halides; aromatic polyesters; and mixtures thereof.

In certain embodiments, the quaternizing agent may be a polyepoxide. Polyepoxides can include, for example: poly-glycidyls, which can include, for example, di-epoxyoctane; ethylene glycol diglycidyl ether; neopentyl glycol digycidyl ether; 1,4-butanediol diglycidyl ether; 3-(bis(glycidyl oxymethyl)methoxy)-1,2-propanediol; 1,4-cyclohexane dimethanol digylicidyl ether; diepoxycyclo-octane; bisphenol A diglycidyl ether; 4-vinyl-1-cyclohexene diepoxide; N,N-Diglycidyl-4,4-glycidyloxyaniline; 1,6-hexane diglycidyl ether; trimethylolpropanetriglycidyl ether; polypropyleneglycol diglycidyl ether; polyepoxidized tri-glycerides, fats or oils; and mixtures thereof.

In certain embodiments, the quaternizing agent may be derived from polyhalides, such as, for example, chlorides, iodides, or bromides. Such polyhalides may include, but are not be limited to: 1,5-dibromopentane; 1,4-diiodobutane; 1,5-dichloropentane; 1,12-dichlorododecane; 1,12-dibromododecane; 1,2-diiodoethane; 1,2-dibromoethane; and mixtures thereof.

In certain embodiments, the quaternizing agent may be an epoxy-halide, such as, for example, epichlorohydrin and the like.

In certain embodiments, the quaternizing agent may be a poly aromatic ester. Examples of poly aromatic esters may include, but are not be limited to: 4,4′-oxybis(methylbenzoate); dimethylterephthalate; and mixtures thereof.

In certain embodiments the molar ratio of the quaternizable compound to quaternizing agent is 1:0.1 to 2, or 1:1 to 1.5, or 1:1 to 1.3. In certain embodiments, such as when employing a coupling quaternizing agent, the ratio of the quaternizable compound to the quaternizing agent may be from 2:1 to 1:1.

In certain embodiments, the quaternizing agent may be employed in the presence of a protic solvent, such as, for example, 2-ethylhexanol, water, and combinations thereof. In certain embodiments, the quaternizing agent may be employed in the presence of an acid. In certain embodiments, the quaternizing agent may be employed in the presence of an acid and a protic solvent.

### Compositions

Also provided are compositions comprising the reaction product comprising a di-hydrocarbyl succinic quaternary ammonium salt. In certain embodiments, the compositions may further comprise at least one other additive. In certain embodiments, the at least one other additive may be at least one of a detergent, a demulsifier, a lubricating agent, a cold flow improver, or an antioxidant, provided that the at least one other additive is not the reaction product comprising a quaternary ammonium salt.

In certain embodiments, the at least one other additive comprises at least one hydrocarbyl-substituted succinic acid and/or at least one hydrocarbyl-substituted quaternary ammonium salt (other than the reaction product comprising a quaternary ammonium salt). The hydrocarbyl-substituent may be a polyisobutylene having a number average molecular weight ranging from 100 to 5000.

Number average molecular weights (Mₙ) described herein may be measured using gel permeation chromatography (GPC) using a Waters GPC 2000 equipped with a refractive index detector and Waters Empower™ data acquisition and analysis software. Samples were run against polystyrene calibration standards. The columns are polystyrene (PLgel, 5 micron, available from Agilent/Polymer Laboratories, Inc.). For the mobile phase, individual samples are dissolved in tetrahydrofuran and filtered with PTFE filters before they are injected into the GPC port. Waters GPC 2000 Operating Conditions: Injector, Column, and Pump/Solvent compartment temperatures, 40° C.; Autosampler Control: Run time, 40 minutes; Injection volume, 300 microliter; Pump, System pressure, ~90 bars (maximum pressure limit, 270 bars; minimum pressure limit, 0 psi); Flow rate, 1.0 ml/minute; Differential Refractometer, Sensitivity, −16; Scale factor: 6.

In certain embodiments, the at least one other additive comprises at least one detergent/dispersant that is an amphiphilic substance which possesses at least one hydrophobic hydrocarbon radical with a number average molecular weight of 100 to 10,000 and at least one polar moiety selected from: (i) Monoamino or polyamino groups having up to 6 nitrogen atoms, at least one nitrogen atom having basic properties; (ii) Hydroxyl groups in combination with monoamino or polyamino groups, at least one nitrogen atoms having basic properties; (iii) Polyoxy-C₂ to C₄ alkylene moieties terminated by hydroxyl groups, monoamino or polyamino groups, at least one nitrogen atom having basic properties, or by carbamate groups; (iv) Moieties derived from succinic anhydride and having hydroxyl and/or amino and/or amido and/or imido groups; and/or (v) Moieties obtained by Mannich reaction of substituted phenols with aldehydes and mono- or polyamines. In certain embodiments, the at least one other additive may comprise at least one Mannich compound.

In certain embodiments, the present technology provides a composition comprising the reaction product comprising a di-hydrocarbyl succinic quaternary ammonium salt described herein, and the use of the composition in a fuel composition to improve water shedding of the fuel composition. In certain embodiments, the present technology provides a composition comprising the reaction product comprising a di-hydrocarbyl succinic quaternary ammonium salt described herein, and the use of the composition in a lubricating composition with an oil of lubricating viscosity. In certain embodiments, the fuel and/or lubricant compositions may additionally comprise at least one additional additive. Such additional additives may be added to any of the compositions described herein, depending on the results desired and/or the application in which the composition will be used. When using the term “additional additive” or “additional additives” herein, what is meant (unless context would require otherwise) is at least one additive that is not (and/or does not comprise) the reaction product comprising a di-hydrocarbyl succinic quaternary ammonium salt as described herein.

Although any of the additional additives described herein could possibly be used in any of the fuel and/or lubricant compositions of the invention (depending on the results desired and/or the application in which the composition will be used), the following additional additives are particularly useful for fuel and/or lubricant compositions: antioxidants, corrosion inhibitors, detergent and/or dispersant additives other than the reactant products comprising a di-hydrocarbyl succinic quaternary ammonium salt described herein, cold flow improvers, foam inhibitors, demulsifiers, lubricity agents, metal deactivators, valve seat recession additives, biocides, antistatic agents, deicers, fluidizers, combustion improvers, seal swelling agents, wax control polymers, scale inhibitors, gas-hydrate inhibitors, or any combination(s) thereof.

Demulsifiers suitable for use as an additional additive may include, but are not limited to, arylsulfonates and polyalkoxylated alcohol, such as, for example, polyethylene and polypropylene oxide copolymers and the like. The demulsifiers can also comprise nitrogen containing compounds such as oxazoline and imidazoline compounds and fatty amines, as well as Mannich compounds. Mannich compounds are the reaction products of alkylphenols and aldehydes (such as formaldehyde) and amines (such as amine condensates and polyalkylenepolyamines). The materials described in the following U.S. Patents are illustrative: U.S. Pat. Nos. 3,036,003; 3,236,770; 3,414,347; 3,448,047; 3,461,172; 3,539,633; 3,586,629; 3,591,598; 3,634,515; 3,725,480; 3,726,882; and 3,980,569. Other suitable demulsifiers are, for example, the alkali metal or alkaline earth metal salts of alkyl-substituted phenol- and naphthalenesulfonates and the alkali metal or alkaline earth metal salts of fatty acids, and also neutral compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example including in the form of EO/PO block copolymers, polyethyleneimines or else polysiloxanes. Any commercially available demulsifiers may be employed, suitably in an amount sufficient to provide a treat level of from 1 to 50 ppm (such as from 5 to 50 ppm) in the fuel. In certain embodiments, there is no demulsifier present as an additional additive in the fuel and/or lubricant composition. The demulsifiers may be used alone or in combination.

Suitable antioxidants for use as additional additives include, for example, hindered phenols or derivatives thereof and/or diarylamines or derivatives thereof. Suitable detergents/dispersants for use as additional additives include, for example, polyetheramines or nitrogen containing detergents, including but not limited to PIB amine detergents/dispersants, succinimide detergents/dispersants, and other quaternary salt detergents/dispersants including polyisobutylsuccinimide-derived quaternized PIB/amine and/or amide dispersants/detergents. Suitable cold flow improvers for use as additional additives include, for example, esterified copolymers of maleic anhydride and styrene and/or copolymers of ethylene and vinyl acetate. Suitable lubricity improvers or friction modifiers for use as additional additives may be based on fatty acids or reaction products of fatty acids, including, for example, fatty acid esters. Examples include tall oil fatty acid, as described, for example, in WO 98/004656, and glyceryl monooleate. The reaction products described in U.S. Pat. No. 6,743,266 B2 of natural or synthetic oils, for example triglycerides, and alkanolamines, are also suitable as such lubricity improvers. Additional examples include commercial tall oil fatty acids containing polycyclic hydrocarbons and/or rosin acids.

Suitable metal deactivators for use as additional additives include, for example, aromatic triazoles or derivatives thereof, including but not limited to benzotriazole. Other suitable metal deactivators include, for example, salicylic acid derivatives such as N,N′-disalicylidene-1,2-propanediamine. Suitable valve seat recession additives for use as additional additives include, for example, alkali metal sulfosuccinate salts. Suitable foam inhibitors and/or antifoams for use as additional additives include, for example, organic silicones (such as polydimethyl siloxane, polyethylsiloxane, polydiethylsiloxane, trimethyl-triflouro-propylmethyl siloxane), polyacrylates, polymethacrylates, polyacrylamides, fluoroacrylates, and the like. Suitable fluidizers for use as additional additives include, for example, mineral oils and/or poly(alpha-olefins) and/or polyethers. Combustion improvers suitable for use as additional additives include, for example, octane and cetane improvers. Suitable cetane number improvers for use as additional additives include, for example, aliphatic nitrates such as 2-ethylhexyl nitrate and cyclohexyl nitrate and peroxides such as di-tert-butyl peroxide.

The additional additives may also include di-ester, di-amide, ester-amide, and ester-imide friction modifiers prepared by reacting an α-hydroxy acid with an amine and/or alcohol optionally in the presence of a known esterification catalyst. Examples of α-hydroxy acids include glycolic acid, lactic acid, α-hydroxy dicarboxylic acid (such as tartaric acid) and/or an α-hydroxy tricarboxylic acid (such as citric acid), with an amine and/or alcohol, optionally in the presence of a known esterification catalyst. These friction modifiers, often derived from tartaric acid, citric acid, or derivatives thereof, may be derived from amines and/or alcohols that are branched, resulting in friction modifiers that themselves have significant amounts of branched hydrocarbyl groups present within it structure. Examples of suitable branched alcohols used to prepare such friction modifiers include 2-ethylhexanol, isotridecanol, Guerbet alcohols, and mixtures thereof. Friction modifiers may be present at 0 to 6 wt % or 0.001 to 4 wt %, or 0.01 to 2 wt % or 0.05 to 3 wt % or 0.1 to 2 wt % or 0.1 to 1 wt % or 0.001 to 0.01 wt %, based on the total weight of the composition.

The additional additives may comprise a detergent/dispersant comprising a hydrocarbyl substituted acylating agent. The acylating agent may be, for example, a hydrocarbyl substituted succinic acid or anhydride, or the reaction product of a hydrocarbyl substituted succinic acid or anhydride with an amine or an alcohol; that is, a hydrocarbyl substituted succinimide or hydrocarbyl substituted succinate. In certain embodiments, the detergent/dispersant may be a polyisobutenyl substituted succinic acid, amide or ester, wherein the polyisobutenyl substituent has a number average molecular weight of 100 to 5000. In certain embodiments, the detergent may be a C₆ to C₁₈ substituted succinic acid, amide or ester. A more thorough description of the hydrocarbyl substituted acylating agent detergents can be found in paragraphs [0017] to [0036] of US 2011/0219674 A1.

In certain embodiments, the detergent/dispersant used as an additional additive may be quaternary ammoniums salts other than those found in the reaction product of the present technology. These different quaternary ammoniums salts may be quaternary ammoniums salts prepared from hydrocarbyl substituted acylating agents, such as, for example, polyisobutyl succinic acids or anhydrides, having a hydrocarbyl substituent with a number average molecular weight of greater than 1200, polyisobutyl succinic acids or anhydrides, having a hydrocarbyl substituent with a number average molecular weight of 300 to 750, or polyisobutyl succinic acids anhydrides, having a hydrocarbyl substituent with a number average molecular weight of 1000. In certain embodiments, such quaternary ammonium salts, prepared from the reaction of nitrogen containing compound and a hydrocarbyl substituted acylating agent having a hydrocarbyl substituent with a number average molecular weight of 1300 to 3000 may be an imide. In certain embodiments, such quaternary ammonium salts, prepared from the reaction of nitrogen containing compound and a hydrocarbyl substituted acylating agent having a hydrocarbyl substituent with a number average molecular weight of greater than 1200 or having a hydrocarbyl substituent with a number average molecular weight of 300 to 750 may be an amide or ester. In certain embodiments, the hydrocarbyl substituted acylating agent may include a mono-, dimer or trimer carboxylic acid with 8 to 54 carbon atoms and is reactive with primary or secondary amines. Suitable acids include, but are not limited to, the mono-, dimer, or trimer acids of caprylic acid, capric acid, lauric acid, myristic acid, palmitic acid, stearic, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid. The hydrocarbyl substituted acylating agent may also be a copolymer formed by copolymerizing at least one monomer that is an ethylenically unsaturated hydrocarbon having 2 to 100 carbon atoms. The monomer may be linear, branched, or cyclic. The monomer may have oxygen or nitrogen substituents, but will not react with amines or alcohols. The monomer may be reacted with a second monomer that is a carboxylic acid or carboxylic acid derivative having 3 to 12 carbon atoms. The second monomer may have one or two carboxylic acid functional groups and is reactive with amines or alcohols. When made using this process, the hydrocarbyl substituted acylating agent copolymer has a number average molecular weight of 500 to 20,000. Alternatively, the hydrocarbyl substituted acylating agent may be a terpolymer that is the reaction product of ethylene and at least one monomer that is an ethylenically unsaturated monomer having at least one tertiary nitrogen atom, with (i) an alkenyl ester of one or more aliphatic monocarboxylic acids having 1 to 24 carbon atoms or (ii) an alkyl ester of acrylic or methacrylic acid. In certain embodiments, the nitrogen containing compound of the different quaternary ammonium salts may be an imidazole or nitrogen containing compound of either of formulas

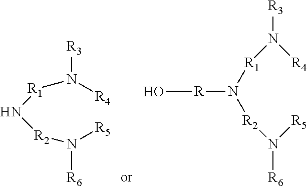

wherein R may be a C₁ to C₆ alkylene group; each of R₁ and R₂, individually, may be a C₁ to C₆ hydrocarbylene group; and each of R₃, R₄, R₅, and R₆, individually, may be a hydrogen or a C₁ to C₆ hydrocarbyl group. In certain embodiments, R₁ or R₂ may be, for example, a C₁, C₂ or C₃ alkylene group. In certain embodiments, each R₃, R₄, R₅, R₆ may be, for example, H or a C₁, C₂ or C₃ alkyl group. In certain embodiments, the quaternizing agent used to prepare the different quaternary ammonium salts can be a dialkyl sulfate, an alkyl halide, a hydrocarbyl substituted carbonate, a hydrocarbyl epoxide, a carboxylate, alkyl esters, aryl esters, or mixtures thereof. In certain embodiments, the quaternizing agent may be a hydrocarbyl epoxide. In certain embodiments, the quaternizing agent may be a hydrocarbyl epoxide in combination with an acid. In certain embodiments, the quaternizing agent may be a salicylate, oxalate or terephthalate. In certain embodiments, the hydrocarbyl epoxide may be an alcohol functionalized epoxide or C₄ to C₁₄ epoxide. In certain embodiments, the hydrocarbyl epoxide may be an alcohol functionalized epoxide or C₄ to C₂₀ epoxide. In certain embodiments, the quaternizing agent is multi-functional resulting in the additional quaternary ammonium salts being a coupled quaternary ammoniums salts. The different quaternary ammonium salts may include, but are not limited to, quaternary ammonium salts having a hydrophobic moiety in the anion. Exemplary compounds include quaternary ammonium compounds having the formula below:

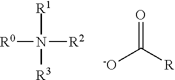

wherein R⁰, R¹, R² and R³ is each individually an optionally substituted alkyl, alkenyl or aryl group and R includes an optionally substituted hydrocarbyl moiety having at least 5 carbon atoms. The different quaternary ammonium salts may also include polyetheramines that are the reaction products of a polyether-substituted amine comprising at least one tertiary quaternizable amino group and a quaternizing agent that converts the tertiary amino group to a quaternary ammonium group.

In certain embodiments, dispersants may be post-treated by reaction with any of a variety of agents. Among these are urea, thiourea, dimercaptothiadiazoles, carbon disulfide, aldehydes, ketones, carboxylic acids, hydrocarbon-substituted succinic anhydrides, nitriles, epoxides, boron compounds, and phosphorus compounds. References detailing such treatment are listed in U.S. Pat. No. 4,654,403.

The fuel and/or lubricant compositions described herein may include a detergent as an additional additive. Many detergents used in the field of engine lubrication obtain most or all of their basicity or total base number (“TBN”) from the presence of basic metal-containing compounds (metal hydroxides, oxides, or carbonates, such as based on such metals as calcium, magnesium, or sodium). Such metallic overbased detergents, also referred to as overbased or superbased salts, are generally single phase, homogeneous Newtonian systems characterized by a metal content in excess of that which would be present for neutralization according to the stoichiometry of the metal and the particular acidic organic compound reacted with the metal. The overbased materials may be prepared by reacting an acidic material (such as an inorganic acid or lower carboxylic acid such as carbon dioxide) with a mixture of an acidic organic compound (also referred to as a substrate), a stoichiometric excess of a metal base, perhaps in a reaction medium of an inert, organic solvent (e.g., mineral oil, naphtha, toluene, xylene) for the acidic organic substrate. In certain embodiments, a small amount of promoter, such as a phenol or alcohol, is present, and in some cases a small amount of water. The acidic organic substrate may have a sufficient number of carbon atoms to provide a degree of solubility in oil. Such overbased materials and their methods of preparation are well known to those skilled in the art. Patents describing techniques for making basic metallic salts of sulfonic acids, carboxylic acids, phenols, phosphonic acids, and mixtures of any two or more of these include U.S. Pat. Nos. 2,501,731; 2,616,905; 2,616,911; 2,616,925; 2,777,874; 3,256,186; 3,384,585; 3,365,396; 3,320,162; 3,318,809; 3,488,284; and 3,629,109. Salixarate detergents are described in U.S. Pat. No. 6,200,936. In certain embodiments, the detergent may contain a metal-containing salicylate detergent, such as an overbased calcium hydrocarbyl-substituted salicylate detergent, which are described in U.S. Pat. Nos. 5,688,751 and 4,627,928.

Viscosity improvers (also sometimes referred to as viscosity index improvers or viscosity modifiers) may be used as an additional additive in the compositions described herein. Viscosity improvers may be polymers, including polyisobutenes, polymethacrylates (“PMA”) and polymethacrylic acid esters, hydrogenated diene polymers, polyalkylstyrenes, esterified styrene-maleic anhydride copolymers, hydrogenated alkenylarene-conjugated diene copolymers and polyolefins. PMAs are prepared from mixtures of methacrylate monomers having different alkyl groups. The alkyl groups may be either straight chain or branched chain groups containing from 1 to 18 carbon atoms. Most PMAs are viscosity modifiers as well as pour point depressants.

Multifunctional viscosity improvers, which also have dispersant and/or antioxidancy properties, are known and may optionally be used in the fuel and/or lubricant compositions as additional additives. Dispersant viscosity modifiers (“DVM”) are one example of such multifunctional additives. DVM may be prepared by copolymerizing a small amount of a nitrogen-containing monomer with alkyl methacrylates, resulting in an additive with some combination of dispersancy, viscosity modification, pour point depressancy, and/or dispersancy. Vinyl pyridine, N-vinyl pyrrolidone and N,N′-dimethylaminoethyl methacrylate are examples of nitrogen-containing monomers. Polyacrylates obtained from the polymerization or copolymerization of one or more alkyl acrylates also are useful as such viscosity modifiers.

Anti-wear agents may be used as additional additives in the fuel and/or lubricant compositions described herein. Anti-wear agents may include phosphorus-containing antiwear/extreme pressure agents, such as metal thiophosphates, phosphoric acid esters and salts thereof, phosphorus-containing carboxylic acids, esters, ethers, and amides, and phosphites. In certain embodiments, a phosphorus antiwear agent may be present in an amount to deliver 0.01 to 0.2, or 0.015 to 0.15, or 0.02 to 0.1, or 0.025 to 0.08 percent by weight phosphorus. The antiwear agent may be a zinc dialkyldithiophosphate (ZDP). For certain ZDPs, which may contain 11 percent P, suitable amounts may include 0.09 to 0.82 percent by weight. Non-phosphorus-containing anti-wear agents include borate esters (including borated epoxides), dithiocarbamate compounds, molybdenum-containing compounds, and sulfurized olefins. In certain embodiments, the fuel and/or lubricant compositions of the invention are free of phosphorus-containing antiwear/extreme pressure agents.

Foam inhibitors that may be useful as additional additives in fuel and/or lubricant compositions described herein include polysiloxanes, copolymers of ethyl acrylate and 2-ethylhexylacrylate, and optionally vinyl acetate; demulsifiers including fluorinated polysiloxanes, trialkyl phosphates, polyethylene glycols, polyethylene oxides, polypropylene oxides, and (ethylene oxide-propylene oxide) polymers. In certain embodiments, the composition(s) may comprise a silicone-containing antifoam agent in combination with a C₅-C₁₇ alcohol.

Pour point depressants that may be useful as additional additives in fuel and/or lubricant compositions described herein include polyalphaolefins, esters of maleic anhydride-styrene copolymers, poly(meth)acrylates, polyacrylates, and/or polyacrylamides.

Metal deactivators may be chosen from a derivative of benzotriazole (such as tolyltriazole), 1,2,4-triazole, benzimidazole, 2-alkyldithiobenzimidazole, or 2-alkyldithiobenzothiazole, 1-amino-2-propanol, a derivative of dimercaptothiadiazole, octylamine octanoate, condensation products of PIBSA or dodecenyl succinic anhydride, and/or a fatty acid such as oleic acid with a polyamine. Metal deactivators may also be described as corrosion inhibitors.

Seal swell agents which may be used as additional additives in the compositions described herein may include sulfolene derivatives, commercially available from Exxon under the trade names Necton-37™ (FN 1380) and Mineral Seal Oil™ (FN 3200).

### Fuel Compositions

Also provided are fuel compositions comprising a fuel and the reaction product comprising a quaternary ammonium salt, and optionally further comprising at least one of the additional additives described above. In certain embodiments, the fuel may be a diesel fuel. In certain embodiments, the fuel may be gasoline. In certain embodiments, the reaction product comprising a quaternary ammonium salt may be added to a fuel composition in an amount of from 5 to 1,000 (such as from 5 to 750, from 5 to 500, from 5 to 250, from 5 to 200, from 5 to 150, from 5 to 100, from 10 to 1,000, from 10 to 750, from 10 to 500, from 10 to 250, from 10 to 200, from 10 to 150, from 10 to 100, from 15 to 1,000, from 15 to 750, from 15 to 500, from 15 to 250, from 15 to 200, from 15 to 150, from 15 to 100, from 20 to 1,000, from 20 to 750, from 20 to 500, from 20 to 250, from 20 to 200, from 20 to 150, from 20 to 100, from 25 to 1,000, from 25 to 750, from 25 to 500, from 25 to 250, from 25 to 200, from 25 to 150, or from 25 to 100) ppm by weight, based on the total weight of the fuel composition.

Also provided are methods of improving water shedding (i.e., demulsification) performance of a fuel composition, the method comprising incorporating the compositions described above into the fuel composition. Also provided is the use of the compositions described above to provide improved water shedding performance in a fuel, such as a fuel that is liquid at room temperature.

Also provided are methods of reducing and/or preventing injector deposits in an engine, the methods comprising supplying a fuel composition as described herein to a fuel injector of the engine and operating the engine.

Also provided are uses of the fuel compositions described herein to reduce and/or prevent internal deposits in an engine operated with the fuel compositions.

In certain embodiments, the fuel composition may comprise a fuel which is liquid at room temperature and is useful in fueling an engine. The fuel may be a liquid at ambient conditions e.g., room temperature (20 to 30° C.). The fuel may be a hydrocarbon fuel, a nonhydrocarbon fuel, or a mixture thereof. The hydrocarbon fuel may be obtained by refining or optionally (hydro)processing a crude petroleum source. It may be a single stream from a refinery stream or a blend of different fractions.

In certain embodiments, the fuel is a gasoline, and in other embodiments the fuel is a leaded gasoline or a nonleaded gasoline. In certain embodiments, the fuel is a diesel fuel. The hydrocarbon fuel may be a hydrocarbon prepared by a process such as the Fischer-Tropsch process. Examples of such fuels include gas to liquid (GTL) process, biomass to liquid (BTL) process, and coal to liquid (CTL) process. The hydrocarbon portion may alternatively be made by hydrocracking or hydrogenation of a vegetable oil. These hydrocarbon fuels may be used alone or in mixtures with other hydrocarbon or nonhydrocarbon fuels. The hydrocarbon fuel may be a petroleum distillate to include a gasoline as defined by EN228 or ASTM specification D4814, or a diesel fuel as defined by EN590 or ASTM specification D975.

The nonhydrocarbon fuel may be an oxygen containing composition, often referred to as an oxygenate, to include an alcohol, an ether, a ketone, an ester of a carboxylic acid, a nitroalkane, or a mixture thereof. The nonhydrocarbon fuel may include for example methanol, ethanol, methyl t-butyl ether, methyl ethyl ketone, transesterified oils and/or fats from plants and animals such as rapeseed methyl ester and soybean methyl ester, and nitromethane. Mixtures of hydrocarbon and nonhydrocarbon fuels may include, for example, gasoline and methanol and/or ethanol, diesel fuel and ethanol, and diesel fuel and a transesterified plant oil (such as rapeseed methyl ester).

In certain embodiments, the liquid fuel is an emulsion of water in a hydrocarbon fuel, a nonhydrocarbon fuel, or a mixture thereof. In certain embodiments, the fuel can have a sulfur content on a weight basis that is 5000 ppm or less, 1000 ppm or less, 300 ppm or less, 200 ppm or less, 30 ppm or less, or 10 ppm or less. In certain embodiments, the fuel can have a sulfur content on a weight basis of 1 to 100 ppm. In certain embodiments, the fuel contains 0 ppm to 1000 ppm, or 0 to 500 ppm, or 0 to 100 ppm, or 0 to 50 ppm, or 0 to 25 ppm, or 0 to 10 ppm, or 0 to 5 ppm of alkali metals, alkaline earth metals, transition metals or mixtures thereof. In certain embodiments, the fuel contains 1 to 10 ppm by weight of alkali metals, alkaline earth metals, transition metals or mixtures thereof. It is well known in the art that a fuel containing alkali metals, alkaline earth metals, transition metals or mixtures thereof may have a greater tendency to form deposits and therefore foul or plug common rail injectors. The fuel of the invention is present in a fuel composition in a major amount that is generally greater than 50 percent by weight, and in certain embodiments is present at greater than 90 percent by weight, greater than 95 percent by weight, greater than 99.5 percent by weight, or greater than 99.8 percent by weight.

Treat rates of the reaction product comprising a quaternary ammonium salt to fuel compositions may range from 5 to 750 ppm, from 5 to 1000 ppm, or 5 to 500 ppm, or 10 to 250 ppm, or 10 to 150 ppm, or 15 to 100 ppm, based on the total weight of the fuel compositions. In certain embodiments, the treat rate range may be from 250 to 1000 ppm, or 250 to 750 ppm, or 500 to 750 ppm, or 250 ppm to 500 ppm, based on the total weight of the fuel compositions.

In certain embodiments, the fuel compositions may further comprise at least one additional additive, as described above. In certain embodiments, the at least one additional additive may comprise at least one demulsifier. Suitable demulsifiers may include, but are not limited to, arylsulfonates and polyalkoxylated alcohol, such as, for example, polyethylene and polypropylene oxide copolymers and the like. The demulsifiers may also comprise nitrogen containing compounds such as oxazoline and imidazoline compounds and fatty amines, as well as Mannich compounds. Mannich compounds are the reaction products of alkylphenols and aldehydes (especially formaldehyde) and amines (especially amine condensates and polyalkylenepolyamines). The materials described in the following U.S. Patents are illustrative: U.S. Pat. Nos. 3,036,003; 3,236,770; 3,414,347; 3,448,047; 3,461,172; 3,539,633; 3,586,629; 3,591,598; 3,634,515; 3,725,480; 3,726,882; and 3,980,569. Other suitable demulsifiers are, for example, the alkali metal or alkaline earth metal salts of alkyl-substituted phenol- and naphthalenesulfonates and the alkali metal or alkaline earth metal salts of fatty acids, and also neutral compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example including in the form of EO/PO block copolymers, polyethyleneimines or else polysiloxanes. Suitable demulsifiers also include a hydrocarbyl-substituted dicarboxylic acid in the form of the free acid, or in the form of the anhydride which may be an intramolecular anhydride, such as succinic, glutaric, or phthalic anhydride, or an intermolecular anhydride linking two dicarboxylic acid molecules together. The hydrocarbyl substituent may have from 12 to 2000 carbon atoms and may include polyisobutenyl substituents having a number average molecular weight of 300 to 2800. Exemplary hydrocarbyl-substituted dicarboxylic acids include, but are not limited to, hydrocarbyl-substituted acids derived from malonic, succinic, glutaric, adipic, pimelic, suberic, azelaic, sebacic, undecanedioic, dodecanedioic, phthalic, isophthalic, terphthalic, o-, m-, or p-phenylene diacetic, maleic, fumaric, or glutaconic acids. In one embodiment the fuel composition of the invention does not comprise a demulsifier as an additional additive. Suitable treat rates of the demulsifiers to a fuel may range from 0 to 50 (such as from 1 to 50, from 5 to 50, from 1 to 25, from 5 to 25, from 1 to 20, or from 5 to 20) ppm by total weight of the fuel.

In certain embodiments, the at least one additional additive may comprise at least one detergent/dispersant. Suitable detergents/dispersants may include amphiphilic substances which possess at least one hydrophobic hydrocarbon group with a number average molecular weight of 100 to 10000 and at least one of the following moieties: (i) Mono- or polyamino groups having up to 6 nitrogen atoms, at least one nitrogen atom having basic properties; (ii) Hydroxyl groups in combination with mono or polyamino groups, at least one nitrogen atoms having basic properties; (iii) Carboxyl groups or their alkali metal or alkaline earth metal salts; (iv) Sulfonic acid groups or their alkali metal or alkaline earth metal salts; (v) Polyoxy-C₂ to C₄ alkylene moieties terminated by hydroxyl groups, mono- or polyamino groups, at least one nitrogen atom having basic properties, or by carbamate groups; (vi) Carboxylic ester groups; (vii) Moieties derived from succinic anhydride and having hydroxyl and/or amino and/or amido and/or imido groups; and/or (viii) Moieties obtained by Mannich reaction of substituted phenols with aldehydes and mono-or polyamines.

The hydrophobic hydrocarbon radical in these detergents/dispersants, which ensures the adequate solubility in the fuel, has, in certain embodiments, a number average molecular weight of 85 to 20,000, of 1113 to 10,000, or of 300 to 5000. In certain embodiments, the detergents/dispersants have a Mₙ of 300 to 3000, of 500 to 2500, of 700 to 2500, or of 800 to 1500. In certain embodiments, the hydrophobic hydrocarbon radicals may be polypropenyl, polybutenyl, and/or polyisobutenyl radicals, with a number average molecular weight of 300 to 5000, of 300 to 3000, of 500 to 2500, or of 700 to 2500. In certain embodiments, the detergents/dispersants have a number average molecular weight of 800 to 1500.

In certain embodiments, the at least one additional additive may comprise at least one high TBN nitrogen containing detergent/dispersant, such as a succinimide, that is the condensation product of a hydrocarbyl-substituted succinic anhydride with a poly(alkyleneamine). Illustrative succinimide detergents/dispersants are more fully described in U.S. Pat. Nos. 4,234,435 and 3,172,892. Another class of ashless dispersant is high molecular weight esters, prepared by reaction of a hydrocarbyl acylating agent and a polyhydric aliphatic alcohol such as glycerol, pentaerythritol, or sorbitol. Such materials are described in more detail in U.S. Pat. No. 3,381,022.

Nitrogen-containing detergents/dispersants may be the reaction products of a carboxylic acid-derived acylating agent and an amine. The acylating agent can vary from formic acid and its acylating derivatives to acylating agents having high molecular weight aliphatic substituents of up to 5,000, 10,000 or 20,000 carbon atoms. The amino compounds can vary from ammonia itself to amines typically having aliphatic substituents of up to 30 carbon atoms, and up to 11 nitrogen atoms. Suitable acylated amino compounds may be those formed by the reaction of an acylating agent having a hydrocarbyl substituent of at least 8 carbon atoms and a compound comprising at least one primary or secondary amine group. The acylating agent may be a mono- or polycarboxylic acid (or reactive equivalent thereof) for example a substituted succinic, phthalic or propionic acid and the amino compound may be a polyamine or a mixture of polyamines, for example a mixture of ethylene polyamines. Alternatively the amine may be a hydroxyalkyl-substituted polyamine. The hydrocarbyl substituent in such acylating agents may comprise at least 10 carbon atoms. In certain embodiments, the hydrocarbyl substituent may comprise at least 12, for example 30 or 50 carbon atoms. In certain embodiments, it may comprise up to 200 carbon atoms. The hydrocarbyl substituent of the acylating agent may have a number average molecular weight (Mₙ) of 170 to 2800, for example from 250 to 1500. In certain embodiments, the substituent's Mₙ may range from 500 to 1500, or alternatively from 500 to 1100. In certain embodiments, the substituent's Mₙ may range from 700 to 1300. In certain embodiments, the hydrocarbyl substituent may have a number average molecular weight of 700 to 1000, or 700 to 850, or, for example, 750.

Another class of ashless dispersant is Mannich bases. These are materials which are formed by the condensation of a higher molecular weight, alkyl substituted phenol, an alkylene polyamine, and an aldehyde such as formaldehyde and are described in more detail in U.S. Pat. No. 3,634,515.

A useful nitrogen containing dispersant includes the product of a Mannich reaction between (a) an aldehyde, (b) a polyamine, and (c) an optionally substituted phenol. The phenol may be substituted such that the Mannich product has a molecular weight of less than 7500. Optionally, the molecular weight may be less than 2000, less than 1500, less than 1300, or for example, less than 1200, less than 1100, less than 1000. In certain embodiments, the Mannich product has a molecular weight of less than 900, less than 850, or less than 800, less than 500, or less than 400. The substituted phenol may be substituted with up to 4 groups on the aromatic ring. For example it may be a tri or di-substituted phenol. In certain embodiments, the phenol may be a mono-substituted phenol. The substitution may be at the ortho, and/or meta, and/or para position(s). To form the Mannich product, the molar ratio of the aldehyde to amine is from 4:1 to 1:1 or, from 2:1 to 1:1. The molar ratio of the aldehyde to phenol may be at least 0.75:1; such as from 0.75 to 1 to 4:1, from 1:1 to 4:1, or from 1:1 to 2:1. To form the Mannich product, the molar ratio of the phenol to amine may be at least 1.5:1, such as at least 1.6:1, at least 1.7:1, at least 1.8:1, or at least 1.9:1. The molar ratio of phenol to amine may be up to 5:1; for example it may be up to 4:1, or up to 3.5:1. In certain embodiments, it is up to 3.25:1, up to 3:1, up to 2.5:1, up to 2.3:1, or up to 2.1:1.

Other dispersants used as additional additives may include polymeric dispersant additives, which are generally hydrocarbon-based polymers which contain polar functionality to impart dispersancy characteristics to the polymer. An amine may be employed in preparing the high TBN nitrogen-containing dispersant. One or more poly(alkyleneamine)s may be used, and these may comprise one or more poly(ethyleneamine)s having 3 to 5 ethylene units and 4 to 6 nitrogen units. Such materials include triethylenetetramine (TETA), tetraethylenepentamine (TEPA), and pentaethylenehexamine (PEHA). Such materials may be commercially available as mixtures of various isomers containing a range number of ethylene units and nitrogen atoms, as well as a variety of isomeric structures, including various cyclic structures. The poly(alkyleneamine) may likewise comprise relatively higher molecular weight amines known in the industry as ethylene amine still bottoms.

In certain embodiments, the fuel composition may additionally comprise quaternary ammonium salts other than the reaction products comprising a di-hydrocarbyl succinic quaternary ammonium salt described herein. These may include alkylamine quaternary ammonium salts. The other quaternary ammonium salts can comprise (a) a compound comprising (i) at least one tertiary amino group as described above, and (ii) a hydrocarbyl-substituent having a number average molecular weight of 100 to 5000, or 250 to 4000, or 100 to 4000 or 100 to 2500 or 3000; and (b) a quaternizing agent suitable for converting the tertiary amino group of (a)(i) to a quaternary nitrogen, as described above. The other quaternary ammonium salts are more thoroughly described in U.S. Pat. Nos. 7,951,211, issued May 31, 2011, and U.S. Pat. No. 8,083,814, issued Dec. 27, 2011, and U.S. Publication Nos. 2013/0118062, published May 16, 2013, 2012/0010112, published Jan. 12, 2012, 2013/0133243, published May 30, 2013, 2008/0113890, published May 15, 2008, and 2011/0219674, published Sep. 15, 2011, US 2012/0149617 published May 14, 2012, US 2013/0225463 published Aug. 29, 2013, US 2011/0258917 published Oct. 27, 2011, US 2011/0315107 published Dec. 29, 2011, US 2013/0074794 published Mar. 28, 2013, US 2012/0255512 published Oct. 11, 2012, US 2013/0333649 published Dec. 19, 2013, US 2013/0118062 published May 16, 2013, and international publications WO Publication Nos. 2011/141731, published Nov. 17, 2011, 2011/095819, published Aug. 11, 2011, and 2013/017886, published Feb. 7, 2013, WO 2013/070503 published May 16, 2013, WO 2011/110860 published Sep. 15, 2011, WO 2013/017889 published Feb. 7, 2013, WO 2013/017884 published Feb. 7, 2013, WO 2014/202425 A2 published Dec. 24, 2014.

The quaternary ammoniums salts used as additional additives may be quaternary ammoniums salts prepared from hydrocarbyl substituted acylating agents, such as, for example, polyisobutyl succinic acids or anhydrides, having a hydrocarbyl substituent with a number average molecular weight of greater than 1200, polyisobutyl succinic acids or anhydrides, having a hydrocarbyl substituent with a number average molecular weight of 300 to 750, or polyisobutyl succinic acids or anhydrides, having a hydrocarbyl substituent with a number average molecular weight of 1000.

In certain embodiments, the fuel composition may further comprise additional quaternary ammonium salts as additional additives. These additional salts may be an imide prepared from the reaction of a nitrogen containing compound and a hydrocarbyl substituted acylating agent having a hydrocarbyl substituent with a number average molecular weight of 1300 to 3000. In certain embodiments, the quaternary ammonium salts prepared from the reaction of nitrogen containing compound and a hydrocarbyl substituted acylating agent having a hydrocarbyl substituent with a number average molecular weight of greater than 1200, or having a hydrocarbyl substituent with a number average molecular weight of 300 to 750, is an amide or ester.

In certain embodiments, the nitrogen containing compound of these additional quaternary ammonium salts may be an imidazole or nitrogen containing compound of either of formulas:

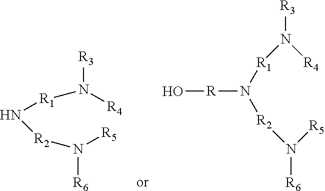

wherein R may be a C₁ to C₆ alkylene group; each of R₁ and R₂, individually, may be a C₁ to C₆ hydrocarbylene group; and each of R₃, R₄, R₅, and R₆, individually, may be a hydrogen or a C₁ to C₆ hydrocarbyl group.

In certain embodiments, the quaternizing agent used to prepare these additional quaternary ammonium salts can be a dialkyl sulfate, an alkyl halide, a hydrocarbyl substituted carbonate, a hydrocarbyl epoxide, a carboxylate, alkyl esters, aryl esters, or mixtures thereof. In certain embodiments, the quaternizing agent can be a hydrocarbyl epoxide. In certain embodiments, the quaternizing agent can be a hydrocarbyl epoxide in combination with an acid. In certain embodiments, the quaternizing agent can be a salicylate, oxalate or terephthalate. In certain embodiments, the hydrocarbyl epoxide is an alcohol functionalized epoxides or C₄ to C₁₄ epoxides.

In certain embodiments, the quaternizing agent is multi-functional resulting in the additional quaternary ammonium salts being a coupled quaternary ammoniums salts.

Illustrative treat rates of additional detergents/dispersants to the fuel compositions described herein is 0 to 500 ppm, or 0 to 250 ppm, or 0 to 100 ppm, or 5 to 250 ppm, or 5 to 100 ppm, or 10 to 100 ppm.

In certain embodiments, the at least one additional additive may comprise at least one cold flow improver. The cold flow improver may be at least one of: (1) copolymers of a C₂- to C₄₀-olefin with at least one further ethylenically unsaturated monomer; (2) comb polymers; (3) polyoxyalkylenes; (4) polar nitrogen compounds; (5) sulfocarboxylic acids or sulfonic acids or derivatives thereof; or (6) poly(meth)acrylic esters. It is possible to use mixtures of different representatives from one of the particular classes (1) to (6) and/or mixtures of representatives from different classes (1) to (6).

Suitable C₂- to C₄₀-olefin monomers for the copolymers of class (1) are, for example, those having 2 to 20, or 2 to 10 carbon atoms, or 1 to 3, or 1 or 2 carbon-carbon double bonds, such as having one carbon-carbon double bond. In the latter case, the carbon-carbon double bond may be arranged either terminally (α-olefins) or internally. Suitable olefin monomers include α-olefins, such as α-olefins having 2 to 6 carbon atoms, for example propene, 1-butene, 1-pentene, 1-hexene, and/or ethylene. The at least one further ethylenically unsaturated monomer of class (1) may be selected from alkenyl carboxylates; for example, C₂- to C₁₄-alkenyl esters, for example the vinyl and propenyl esters, of carboxylic acids having 2 to 21 carbon atoms, whose hydrocarbon radical may be linear or branched among these, such as vinyl esters; examples of suitable alkenyl carboxylates are vinyl acetate, vinyl propionate, vinyl butyrate, vinyl 2-ethylhexanoate, vinyl neopentanoate, vinyl hexanoate, vinyl neononanoate, vinyl neodecanoate and the corresponding propenyl esters, (meth)acrylic esters; for example, esters of (meth)acrylic acid with C₁- to C₂₀-alkanols, especially C₁- to C₁₀-alkanols, such as with methanol, ethanol, propanol, isopropanol, n-butanol, sec-butanol, isobutanol, tert-butanol, pentanol, hexanol, heptanol, octanol, 2-ethylhexanol, nonanol and decanol, and structural isomers thereof and further olefins; such as higher in molecular weight than the abovementioned C₂- to C₄₀-olefin base monomer for example, the olefin base monomer used is ethylene or propene, suitable further olefins are in particular C₁₀- to C₄₀-α-olefins.

Suitable illustrative copolymers of class (1) are also those which comprise two or more different alkenyl carboxylates in copolymerized form, which differ in the alkenyl function and/or in the carboxylic acid group. Likewise suitable are copolymers which, as well as the alkenyl carboxylate(s), comprise at least one olefin and/or at least one (meth)acrylic ester in copolymerized form.

Terpolymers of a C₂- to C₄₀-α-olefin, a C₁- to C₂₀-alkyl ester of an ethylenically unsaturated monocarboxylic acid having 3 to 15 carbon atoms and a C₂- to C₁₄-alkenyl ester of a saturated monocarboxylic acid having 2 to 21 carbon atoms are also suitable as copolymers of class (1). Terpolymers of this kind are described in WO 2005/054314. An illustrative terpolymer of this kind is formed from ethylene, 2-ethylhexyl acrylate and vinyl acetate.

In certain embodiments, the at least one or the further ethylenically unsaturated monomer(s) are copolymerized in the copolymers of class (1) in an amount of from 1 to 50% by weight, such from 10 to 45% by weight, or from 20 to 40% by weight, based on the overall copolymer. The main proportion in terms of weight of the monomer units in the copolymers of class (1) may therefore originate from the C₂ to C₄₀ base olefins. The copolymers of class (1) may have a number average molecular weight of 1000 to 20,000, or 1000 to 10,000 or 1000 to 8000.

Illustrative comb polymers of component (2) are, for example, obtainable by the copolymerization of maleic anhydride or fumaric acid with another ethylenically unsaturated monomer, for example with an a-olefin or an unsaturated ester, such as vinyl acetate, and subsequent esterification of the anhydride or acid function with an alcohol having at least 10 carbon atoms. Further suitable comb polymers are copolymers of α-olefins and esterified comonomers, for example esterified copolymers of styrene and maleic anhydride or esterified copolymers of styrene and fumaric acid. Suitable comb polymers may also be polyfumarates or polymaleates. Homo- and copolymers of vinyl ethers are also suitable comb polymers. Comb polymers suitable as components of class (2) are, for example, also those described in WO 2004/035715 and in “Comb-Like Polymers. Structure and Properties”, N. A. Plate and V. P. Shibaev, J. Poly. Sci. Macromolecular Revs. 8, pages 117 to 253 (1974). Mixtures of comb polymers are also suitable.

Polyoxyalkylenes suitable as components of class (3) are, for example, polyoxyalkylene esters, polyoxyalkylene ethers, mixed polyoxyalkylene ester/ethers and mixtures thereof. These polyoxyalkylene compounds may comprise at least one linear alkyl group, such as at least two linear alkyl groups, each having 10 to 30 carbon atoms and a polyoxyalkylene group having a number average molecular weight of up to 5000. Such polyoxyalkylene compounds are described, for example, in EP-A 061 895 and also in U.S. Pat. No. 4,491,455. Suitable polyoxyalkylene compounds are based on polyethylene glycols and polypropylene glycols having a number average molecular weight of 100 to 5000. Additionally suitable are polyoxyalkylene mono- and diesters of fatty acids having 10 to 30 carbon atoms, such as stearic acid or behenic acid.

Polar nitrogen compounds suitable as components of class (4) may be either ionic or nonionic and may have at least one substituent, or at least two substituents, in the form of a tertiary nitrogen atom of the general formula>NR⁷ in which R⁷ is a C₈- to C₄₀-hydrocarbon radical. The nitrogen substituents may also be quaternized, i.e. be in cationic form. An example of such nitrogen compounds is that of ammonium salts and/or amides which are obtainable by the reaction of at least one amine substituted by at least one hydrocarbon radical with a carboxylic acid having 1 to 4 carboxyl groups or with a suitable derivative thereof. The amines may comprise at least one linear C₈- to C₄₀-alkyl radical. Primary amines suitable for preparing the polar nitrogen compounds mentioned are, for example, octylamine, nonylamine, decylamine, undecylamine, dodecylamine, tetradecylamine and the higher linear homologs. Secondary amines suitable for this purpose are, for example, dioctadecylamine and methylbehenylamine. Also suitable for this purpose are amine mixtures, in particular amine mixtures obtainable on the industrial scale, such as fatty amines or hydrogenated tallamines, as described, for example, in Ullmann's Encyclopedia of Industrial Chemistry, 6th Edition, “Amines, aliphatic” chapter. Acids suitable for the reaction are, for example, cyclohexane-1,2-dicarboxylic acid, cyclohexene-1,2-dicarboxylic acid, cyclopentane-1,2-dicarboxylic acid, naphthalene dicarboxylic acid, phthalic acid, isophthalic acid, terephthalic acid, and succinic acids substituted by long-chain hydrocarbon radicals.

Sulfocarboxylic acids, sulfonic acids or derivatives thereof which are suitable as cold flow improvers of class (5) are, for example, oil-soluble carboxamides and carboxylic esters of ortho-sulfobenzoic acid, in which the sulfonic acid function is present as a sulfonate with alkyl-substituted ammonium cations, as described in EP-A 261 957.

Poly(meth)acrylic esters suitable as cold flow improvers of class (6) are either homo- or copolymers of acrylic and methacrylic esters. Also suitable are copolymers of at least two different (meth)acrylic esters which differ with regard to the esterified alcohol. The copolymer optionally comprises another different olefinically unsaturated monomer in copolymerized form. The weight-average molecular weight of the polymer is preferably 50,000 to 500,000. The polymer may be a copolymer of methacrylic acid and methacrylic esters of saturated C₁₄ and C₁₅ alcohols, the acid groups having been neutralized with hydrogenated tallamine. Suitable poly(meth)acrylic esters are described, for example, in WO 00/44857.

The additional additive comprising at least one cold flow improver may be added to the fuel composition in an amount of from 0 to 5000 ppm by weight, or from 10 to 5000 ppm by weight, or from 20 to 2000 ppm by weight, or from 50 to 1000 ppm by weight, or from 100 to 700 ppm by weight, or from 200 to 500 ppm by weight, based on the total weight of the fuel composition.

### Lubricating Compositions

Also provided are lubricating compositions comprising an oil of lubricating viscosity and the compositions described above. The reaction product comprising a di-hydrocarbyl succinic quaternary ammonium salt may be present in the lubricating composition in an amount of from 1 to 5 percent by weight, based on the total weight of the lubricating composition. In certain embodiments, the oil of lubricating viscosity may have a total sulfated ash level of less than 1 percent by weight, based on the total weight of the lubricating composition. In certain embodiments, the oil of lubricating viscosity may have a total phosphorus content of less than 0.11 percent by weight, based on the total weight of the lubricating composition.

Also provided are methods of lubricating the crankcase of an engine, the methods comprising supplying the lubricating compositions described herein to the engine and operating the engine.

Also provided are uses of the lubricating compositions described herein to lubricate the crankcase of an engine.

Oils of lubricating viscosity may include natural and synthetic oils, oil derived from hydrocracking, hydrogenation, and/or hydrofinishing, unrefined, refined, re-refined oils, or mixtures thereof. A more detailed description of unrefined, refined, and re-refined oils is provided in WO 2008/147704, paragraphs [0054] to [0056]. A more detailed description of natural and synthetic lubricating oils is provided in paragraphs [0058] to [0059] of WO2008/147704. Synthetic oils may also be produced by Fischer-Tropsch reactions and may be hydroisomerized Fischer-Tropsch hydrocarbons or waxes. In certain embodiments, oils may be prepared by a Fischer-Tropsch gas-to liquid synthetic procedure as well as other gas-to-liquid oils.

Oils of lubricating viscosity may also be selected from any of the base oils in Groups I-V as specified in the American Petroleum Institute (API) Base Oil Interchangeability Guidelines. The five base oil groups are as follows; Group I, >0.03% sulfur or <90% saturates, and viscosity index 80-120; Group II, <0.03% sulfur and >90% saturates and viscosity index 80-120; Group III, <0.03% sulfur and >90% saturates and viscosity index>120; Group IV, all polyalphaolefins; Group V, all others. Groups I, II and III may be referred to as mineral oil base stocks.

Illustrative treat rates of the reaction product comprising a di-hydrocarbyl succinic quaternary ammonium salt to lubricating oils is from 0.1 to 10 wt %, or from 0.5 to 5 wt %, or from 0.5 to 2.5 wt %, or from 0.5 to 1 wt %, or from 0.1 to 0.5 wt %, or from 1 to 2 wt %, based on a total weight of the lubricating oil.

The amount of the oil of lubricating viscosity present in the lubricating compositions described herein is the balance remaining after subtracting the total amount of all additives, including the reaction product comprising a di-hydrocarbyl succinic quaternary ammonium salt described herein, and any additional additives as described herein.

The lubricating compositions described herein may be in the form of a concentrate or fully formulated lubricant. If the lubricating composition is in the form of a concentrate (which may be combined with additional oil to from, in whole or in part, a finished lubricant), the ratio of these additive to the oil of lubricating viscosity and/or diluent oil include the ranges of 1:99 to 99:1 by weight, or 80:20 to 10:90 by weight.

In certain embodiments, the lubricating compositions described herein may be employed in internal combustion engines. The internal combustion engine may be spark ignition or compression ignition. The internal combustion engine may be a 2-stroke or 4-stroke engine. The internal combustion engine may be a passenger car engine, a light duty diesel engine, a heavy duty diesel engine, a motorcycle engine, or a 2-stroke or 4-stroke marine diesel engine. The internal combustion engine may be a passenger car engine, or a heavy duty diesel internal combustion engine.

In certain embodiments, a lubricating composition as described herein may comprise, as an additional additive, an overbased metal-containing detergent. Overbased detergents are known in the art. Overbased materials, otherwise referred to as overbased or superbased salts, are generally single phase, homogeneous systems characterized by a metal content in excess of that which would be present for neutralization according to the stoichiometry of the metal and the particular acidic organic compound reacted with the metal. The overbased materials are prepared by reacting an acidic material (such as an inorganic acid or lower carboxylic acid, such as carbon dioxide) with a mixture comprising an acidic organic compound, a reaction medium comprising at least one inert, organic solvent (mineral oil, naphtha, toluene, xylene, etc.) for said acidic organic material, a stoichiometric excess of a metal base, and a promoter such as a calcium chloride, acetic acid, phenol or alcohol. The acidic organic material may have a sufficient number of carbon atoms to provide a degree of solubility in oil. The amount of “excess” metal (stoichiometrically) is commonly expressed in terms of metal ratio. The term “metal ratio” is the ratio of the total equivalents of the metal to the equivalents of the acidic organic compound. A neutral metal salt has a metal ratio of one. A salt having 4.5 times as much metal as present in a normal salt will have metal excess of 3.5 equivalents, or a ratio of 4.5. The term “metal ratio” is also explained in “Chemistry and Technology of Lubricants”, Third Edition, Edited by R. M. Mortier and S. T. Orszulik, Copyright 2010, page 219, sub-heading 7.25.

The overbased metal-containing detergent may be chosen from non-sulfur-containing phenates, sulfur-containing phenates, sulfonates, salixarates, salicylates, carboxylates, and mixtures thereof, or borated equivalents thereof. The overbased detergent may be borated with a borating agent such as boric acid.

The overbased detergent may be non-sulfur containing phenates, sulfur containing phenates, sulfonates, or mixtures thereof.

In certain embodiments, the overbased sulfonate detergent may be present in the lubricating composition in an amount of from 0.01 wt % to 0.9 wt %, or 0.05 wt % to 0.8 wt %, or 0.1 wt % to 0.7 wt %, or 0.2 wt % to 0.6 wt %, based on the total weight of the lubricating composition.

In certain embodiments, the overbased sulfonate detergent may have a metal ratio of 12 to less than 20, or 12 to 18, or 20 to 30, or 22 to 25.

Overbased sulfonates may have a total base number of 250 to 600, or 300 to 500 (on an oil free basis). Overbased detergents are known in the art. In certain embodiments, the sulfonate detergent may be a predominantly linear alkylbenzene sulfonate detergent having a metal ratio of at least 8 as is described in paragraphs [0026] to [0037] of US Patent Application 2005065045. Linear alkyl benzenes may have the benzene ring attached anywhere on the linear chain, such as at the 2, 3, or 4 position, or mixtures thereof. The predominantly linear alkylbenzene sulfonate detergent may be useful for assisting in improving fuel economy. In certain embodiments, the sulfonate detergent may be a metal salt of one or more oil-soluble alkyl toluene sulfonate compounds as disclosed in paragraphs [0046] to [0053] of US Patent Application 2008/0119378.

In certain embodiments, the overbased sulfonate detergent comprises an overbased calcium sulfonate. The calcium sulfonate detergent may have a metal ratio of 18 to 40 and a TBN of 300 to 500, or 325 to 425.

The other detergents may also include “hybrid” detergents formed with mixed surfactant systems including phenate and/or sulfonate components, e.g., phenate/salicylates, sulfonate/phenates, sulfonate/salicylates, sulfonates/phenates/salicylates, as described; for example, in U.S. Pat. Nos. 6,429,178; 6,429,179; 6,153,565; and 6,281,179. Where, for example, a hybrid sulfonate/phenate detergent is employed, the hybrid detergent would be considered equivalent to amounts of distinct phenate and sulfonate detergents introducing like amounts of phenate and sulfonate detergents, respectively.

The other detergents may have an alkali metal, an alkaline earth metal, or zinc counter ion. In certain embodiments, the metal may be sodium, calcium, barium, or magnesium. Other detergents may be sodium, calcium, or magnesium containing detergent.

The other detergent may be an overbased detergent of sodium, calcium or magnesium salt of the phenates, sulfur-containing phenates, salixarates and salicylates. Overbased phenates and salicylates typically have a total base number of 180 to 450 TBN (on an oil free basis).

Phenate detergents may be derived from p-hydrocarbyl phenols. Alkylphenols of this type may be coupled with sulfur and overbased, coupled with aldehyde and overbased, or carboxylated to form salicylate detergents. Suitable alkylphenols include those alkylated with oligomers of propylene, e.g., tetrapropenylphenol (e.g., p-dodecylphenol or PDDP) and pentapropenylphenol. Other suitable alkylphenols include those alkylated with alpha-olefins, isomerized alpha-olefins, and polyolefins like polyisobutylene. In certain embodiments, the lubricating composition comprises less than 0.2 wt %, or less than 0.1 wt %, or less than 0.05 wt % of a phenate detergent derived from PDDP. In certain embodiments, the lubricating composition comprises a phenate detergent that is not derived from PDDP.

The overbased detergent may be present in the lubricating composition in an amount of 0 wt % to 10 wt %, or 0.1 wt % to 10 wt %, or 0.2 wt % to 8 wt %, or 0.2 wt % to 3 wt %, based on the total weight of the lubricating composition. For example, in a heavy duty diesel engine lubricating composition, the detergent may be present at 2 wt % to 3 wt % of the lubricating composition. For a passenger car engine, the detergent may be present in the lubricating composition an amount of 0.2 wt % to 1 wt %, based on the total weight of the lubricating composition. In certain embodiments, the lubricating composition comprises at least one overbased detergent with a metal ratio of at least 3, or at least 8, or at least 15.

In certain embodiments, a lubricating composition comprise at least one dispersant as an additional additive. The dispersant may be chosen from a succinimide dispersant, a Mannich dispersant, a succinamide dispersant, a polyolefin succinic acid ester, amide, or ester-amide, or mixtures thereof.

The succinimide dispersant may be derived from an aliphatic polyamine. The aliphatic polyamine may be an ethylenepolyamine, a propylenepolyamine, a butylenepolyamine, or mixtures thereof. In certain embodiments, the aliphatic polyamine may be chosen from ethylenediamine, diethylenetriamine, triethylenetetramine, tetraethylenepentamine, pentaethylenehexamine, polyamine still bottoms, and mixtures thereof.

In certain embodiments, the dispersant may be a polyolefin succinic acid ester, amide, or ester-amide. For instance, a polyolefin succinic acid ester may be a polyisobutylene succinic acid ester of pentaerythritol, or mixtures thereof. A polyolefin succinic acid ester-amide may be a polyisobutylene succinic acid reacted with an alcohol (such as pentaerythritol) and an amine (such as a diamine, or diethyleneamine).

The dispersant may be an N-substituted long chain alkenyl succinimide. An example of an N-substituted long chain alkenyl succinimide is polyisobutylene succinimide. The polyisobutylene from which polyisobutylene succinic anhydride may be derived may have a number average molecular weight of 350 to 5000, or 550 to 3000 or 750 to 2500. Succinimide dispersants and their preparation are disclosed, for instance, in U.S. Pat. Nos. 3,172,892, 3,219,666, 3,316,177, 3,340,281, 3,351,552, 3,381,022, 3,433,744, 3,444,170, 3,467,668, 3,501,405, 3,542,680, 3,576,743, 3,632,511, 4,234,435, Re 26,433, and 6,165,235, 7,238,650 and EP Patent Application 0 355 895 A.

The dispersants may also be post-treated by conventional methods by a reaction with any of a variety of agents. Among these are boron compounds (such as boric acid), urea, thiourea, dimercaptothiadiazoles, carbon disulfide, aldehydes, and ketones, carboxylic acids such as terephthalic acid, hydrocarbon-substituted succinic anhydrides, maleic anhydride, nitriles, epoxides, and phosphorus compounds. In certain embodiments, the post-treated dispersant is borated. In certain embodiments, the post-treated dispersant may be reacted with dimercaptothiadiazoles. In certain embodiments, the post-treated dispersant may be reacted with phosphoric or phosphorous acid. In certain embodiments, the post-treated dispersant may be reacted with terephthalic acid and boric acid (as described in US Patent Application US2009/0054278).

In certain embodiments, the dispersant may be borated or non-borated. A borated dispersant may be a succinimide dispersant. In certain embodiments, the ashless dispersant may be boron-containing, i.e., has incorporated boron and delivers said boron to the lubricant composition. The boron-containing dispersant may be present in an amount to deliver at least 25 ppm boron, at least 50 ppm boron, or at least 100 ppm boron to the lubricating composition. In certain embodiments, the lubricating composition may be free of a boron-containing dispersant. In certain embodiments, the lubricating composition has no more than 10 ppm boron.

The dispersant which may be used as an additional additive in the lubricating compositions described herein, may be prepared/obtained/obtainable from reaction of succinic anhydride by an “ene” or “thermal” reaction, by what may be referred to as a “direct alkylation process.” The “ene” reaction mechanism and general reaction conditions are summarized in “Maleic Anhydride”, pages, 147-149, Edited by B. C. Trivedi and B. C. Culbertson and Published by Plenum Press in 1982. The dispersant prepared by a process that includes an “ene” reaction may be a polyisobutylene succinimide having a carbocyclic ring present on less than 50 mole %, or 0 to less than 30 mole %, or 0 to less than 20 mole %, or 0 mole % of the dispersant molecules. The “ene” reaction may have a reaction temperature of 180° C. to less than 300° C., or 200° C. to 250° C., or 200° C. to 220° C.

The dispersant may also be obtained/obtainable from a chlorine-assisted process, often involving Diels-Alder chemistry, leading to formation of carbocyclic linkages. The chlorine-assisted process may produce a dispersant that is a polyisobutylene succinimide having a carbocyclic ring present on 50 mole % or more, or 60 to 100 mole % of the dispersant molecules. Both the thermal and chlorine-assisted processes are described in greater detail in U.S. Pat. No. 7,615,521, columns 4-5 and preparative examples A and B thereof.

The dispersant may have a carbonyl to nitrogen ratio (CO:N ratio) of 5:1 to 1:10, 2:1 to 1:10, or 2:1 to 1:5, or 2:1 to 1:2. In certain embodiments, the dispersant may have a CO:N ratio of 2:1 to 1:10, or 2:1 to 1:5, or 2:1 to 1:2, or 1:1.4 to 1:0.6.

In certain embodiments, the dispersant may be a succinimide dispersant, and may comprise a polyisobutylene succinimide, wherein the polyisobutylene from which polyisobutylene succinimide is derived has a number average molecular weight of 350 to 5000, or 750 to 2500. The dispersant may be present in the lubricating composition in an amount of 0 wt % to 20 wt %, 0.1 wt % to 15 wt %, or 0.5 wt % to 9 wt %, or 1 wt % to 8.5 wt % or 1.5 to 5 wt %, based on the total weight of the lubricating composition.

In certain embodiments, the lubricating composition may comprise a molybdenum compound as an additional additive. The molybdenum compound may be an antiwear agent or an antioxidant. The molybdenum compound may be chosen from molybdenum dialkyldithiophosphates, molybdenum dithiocarbamates, amine salts of molybdenum compounds, and mixtures thereof. The molybdenum compound may provide the lubricating composition with 0 to 1000 ppm, or 5 to 1000 ppm, or 10 to 750 ppm, or 5 ppm to 300 ppm, or 20 ppm to 250 ppm of molybdenum.

In certain embodiments, the lubricating composition may further comprise an antioxidant as an additional additive. Antioxidants include sulfurized olefins, diarylamines, alkylated diarylamines, hindered phenols, molybdenum compounds (such as molybdenum dithiocarbamates), hydroxyl thioethers, or mixtures thereof. The antioxidant may be present in the lubricating composition in an amount of 0 wt % to 15 wt %, or 0.1 wt % to 10 wt %, or 0.5 wt % to 5 wt %, or 0.5 wt % to 3 wt %, or 0.3 wt % to 1.5 wt %, based on the total weight of the lubricating composition.

In certain embodiments, the lubricating composition may further comprise a phenolic or an aminic antioxidant or mixtures thereof, as an additional additive, and optionally, the antioxidant may be present at 0.1 wt % to 3 wt %, or 0.5 wt % to 2.75 wt %, or 1 wt % to 2.5 wt %, based on the total weight of the lubricating composition.

The diarylamine or alkylated diarylamine may be a phenyl-α-naphthylamine (PANA), an alkylated diphenylamine, or an alkylated phenylnapthylamine, or mixtures thereof. The alkylated diphenylamine may include di-nonylated diphenylamine, nonyl diphenylamine, octyl diphenylamine, di-octylated diphenylamine, di-decylated diphenylamine, decyl diphenylamine and mixtures thereof. In certain embodiments, the diphenylamine may include nonyl diphenylamine, dinonyl diphenylamine, octyl diphenylamine, dioctyl diphenylamine, or mixtures thereof. In certain embodiments, the alkylated diphenylamine may include nonyl diphenylamine, or dinonyl diphenylamine. The alkylated diarylamine may include octyl, dioctyl, nonyl, di-nonyl, decyl or di-decyl phenylnapthylamines.

The hindered phenol antioxidant may contain a secondary butyl and/or a tertiary butyl group as a sterically hindering group. The phenol group may be further substituted with a hydrocarbyl group (typically linear or branched alkyl) and/or a bridging group linking to a second aromatic group. Examples of suitable hindered phenol antioxidants include 2,6-di-tert-butylphenol, 4-methyl-2,6-di-tert-butylphenol, 4-ethyl-2,6-di-tert-butylphenol, 4-propyl-2,6-di-tert-butylphenol or 4-butyl-2,6-di-tert-butylphenol, or 4-dodecyl-2,6-di-tert-butylphenol. In certain embodiments, the hindered phenol antioxidant may be an ester and may include, e.g., Irganox™ L-135 from Ciba. A more detailed description of suitable ester-containing hindered phenol antioxidant chemistry may be found in U.S. Pat. No. 6,559,105.

Examples of molybdenum dithiocarbamates, which may be used as an antioxidant, include commercial materials sold under the trade names such as Molyvan 822®, Molyvan® A and Molyvan® 855 from R. T. Vanderbilt Co., Ltd., and Adeka Sakura-Lube™ S-100, S-165, S-600 and 525, or mixtures thereof.

In certain embodiments, the lubricating composition may further comprise a viscosity modifier as an additional additive. Viscosity modifiers are known in the art and may include hydrogenated styrene-butadiene rubbers, ethylene-propylene copolymers, ethylene copolymers with propylene and higher olefins, polymethacrylates, polyacrylates, hydrogenated styrene-isoprene polymers, hydrogenated diene polymers, polyalkyl styrenes, polyolefins, esters of maleic anhydride-olefin copolymers (such as those described in International Application WO 2010/014655), esters of maleic anhydride-styrene copolymers, or mixtures thereof. The viscosity modifier may include a block copolymer comprising (i) a vinyl aromatic monomer block, and (ii) a conjugated diene olefin monomer block (such as a hydrogenated styrene-butadiene copolymer or a hydrogenated styrene-isoprene copolymer), a polymethacrylate, an ethylene-alpha olefin copolymer, a hydrogenated star polymer comprising conjugated diene monomers such as butadiene or isoprene, or a star polymer of polymethacrylate, or mixtures thereof.

In certain embodiments, the viscosity modifier may be a dispersant viscosity modifier. The dispersant viscosity modifier may include functionalized polyolefins, for example, ethylene-propylene copolymers that have been functionalized with an acylating agent such as maleic anhydride and an amine.

In certain embodiments, the dispersant viscosity modifier comprises an olefin copolymer further functionalized with a dispersant amine group. The olefin copolymer may be an ethylene-propylene copolymer. The olefin copolymer may have a number average molecular weight of 5000 to 20,000, or 6000 to 18,000, or 7000 to 15,000. The olefin copolymer may have a shear stability index of 0 to 20, or 0 to 10, or 0 to 5 as measured by the Orbahn shear test (ASTM D6278).

The formation of a dispersant viscosity modifier is well known in the art. The dispersant viscosity modifier may include, for instance, those described in U.S. Pat. No. 7,790,661 column 2, line 48 to column 10, line 38.

In certain embodiments, the dispersant viscosity modifier may be prepared by grafting of an olefinic carboxylic acid acylating agent onto a polymer of 15 to 80 mole percent of ethylene, from 20 to 85 mole percent of C₃₋₁₀ α-monoolefin, and from 0 to 15 mole percent of non-conjugated diene or triene, said polymer, and further reacting said grafted polymer with an amine (typically an aromatic amine).

The dispersant viscosity modifier may include functionalized polyolefins, for example, ethylene-propylene copolymers that have been functionalized with an acylating agent such as maleic anhydride and an amine; polymethacrylates functionalized with an amine, or styrene-maleic anhydride copolymers reacted with an amine. Suitable amines may be aliphatic or aromatic amines and polyamines. Examples of suitable aromatic amines include nitroaniline, aminodiphenylamine (ADPA), hydrocarbylene coupled polyaromatic amines, and mixtures thereof. More detailed description of dispersant viscosity modifiers are provided in International Publication WO2006/015130 or U.S. Pat. Nos. 4,863,623; 6,107,257; 6,107,258; 6,117,825; and 7,790,661.

In certain embodiments, the dispersant viscosity modifier may include those described in U.S. Pat. No. 4,863,623 (see column 2, line 15 to column 3, line 52) or in International Publication WO2006/015130 (see page 2, paragraph [0008] and paragraphs [0065] to [0073]).

In certain embodiments, the lubricating compositions disclosed herein may further comprise a dispersant viscosity modifier, which may be present at 0 wt % to 5 wt %, or 0 wt % to 4 wt %, or 0.05 wt % to 2 wt %, or 0.2 wt % to 1.2 wt %, based on the total weight of the lubricating composition.

In certain embodiments, the lubricating composition may further comprise at least one friction modifier as an additional additive. In certain embodiments, the friction modifier may be chosen from long chain fatty acid derivatives of amines, long chain fatty esters, or derivatives of long chain fatty epoxides; fatty imidazolines; amine salts of alkylphosphoric acids; fatty alkyl tartrates; fatty alkyl tartrimides; fatty alkyl tartramides; fatty malic esters and imides, fatty (poly)glycolates; and fatty glycolamides. The friction modifier may be present in the lubricating composition in an amount of 0 wt % to 6 wt %, or 0.01 wt % to 4 wt %, or 0.05 wt % to 2 wt %, or 0.1 wt % to 2 wt %, based on the total weight of the lubricating composition. As used herein the term “fatty alkyl” or “fatty” in relation to friction modifiers means, in certain embodiments, a carbon chain having 10 to 22 carbon atoms, such as a straight carbon chain.

Examples of suitable friction modifiers include long chain fatty acid derivatives of amines, fatty esters, or fatty epoxides; fatty imidazolines such as condensation products of carboxylic acids and polyalkylene-polyamines; amine salts of alkylphosphoric acids; fatty alkyl tartrates; fatty alkyl tartrimides; fatty alkyl tartramides; fatty phosphonates; fatty phosphites; borated phospholipids, borated fatty epoxides; glycerol esters such as glycerol mono-oleate; borated glycerol esters; fatty amines; alkoxylated fatty amines; borated alkoxylated fatty amines; hydroxyl and polyhydroxy fatty amines including tertiary hydroxy fatty amines; hydroxy alkyl amides; metal salts of fatty acids; metal salts of alkyl salicylates; fatty oxazolines; fatty ethoxylated alcohols; condensation products of carboxylic acids and polyalkylene polyamines; or reaction products from fatty carboxylic acids with guanidine, aminoguanidine, urea, or thiourea and salts thereof.

Friction modifiers may also encompass materials such as sulfurized fatty compounds and olefins, molybdenum dialkyldithiophosphates, molybdenum dithiocarbamates, sunflower oil or soybean oil monoester of a polyol and an aliphatic carboxylic acid.

In certain embodiments, the friction modifier may be a long chain fatty acid ester. In certain embodiments, the long chain fatty acid ester may be a mono-ester and/or a triglyceride.

In certain embodiments, the lubricating composition may further comprise at least one antiwear agent as an additional additive. Examples of suitable antiwear agents include titanium compounds, tartaric acid derivatives such as tartrate esters, amides or tartrimides, malic acid derivatives, citric acid derivatives, glycolic acid derivatives, oil soluble amine salts of phosphorus compounds different from that of the invention, sulfurized olefins, metal dihydrocarbyldithiophosphates (such as zinc dialkyldithiophosphates), phosphites (such as dibutyl phosphite), phosphonates, thiocarbamate-containing compounds, such as thiocarbamate esters, thiocarbamate amides, thiocarbamic ethers, alkylene-coupled thiocarbamates, and bis(S-alkyldithiocarbamyl) disulfides.

The antiwear agent may include a tartrate or tartrimide as disclosed in International Publication WO 2006/044411 or Canadian Patent CA 1 183 125. The tartrate or tartrimide may contain alkyl-ester groups, where the sum of carbon atoms on the alkyl groups is at least 8. The antiwear agent may include a citrate as is disclosed in US Patent Application 20050198894.

Another class of additives includes oil-soluble titanium compounds as disclosed in U.S. Pat. No. 7,727,943 and US2006/0014651. The oil-soluble titanium compounds may function as antiwear agents, friction modifiers, antioxidants, deposit control additives, or more than one of these functions. The oil soluble titanium compound may be a titanium (IV) alkoxide. The titanium alkoxide is formed from a monohydric alcohol, a polyol or mixtures thereof. The monohydric alkoxides may have 2 to 16, or 3 to 10 carbon atoms. The titanium alkoxide may be titanium (IV) isopropoxide. The titanium alkoxide may be titanium (IV) 2-ethylhexoxide. The titanium compound may comprise the alkoxide of a vicinal 1,2-diol or polyol. The 1,2-vicinal diol may comprise a fatty acid mono-ester of glycerol, such as where the fatty acid is oleic acid. The oil soluble titanium compound may be a titanium carboxylate. The titanium (IV) carboxylate may be titanium neodecanoate.

In certain embodiments, the lubricating composition may further comprise at least one phosphorus-containing antiwear agent as an additional additive. The phosphorus-containing antiwear agent may be a zinc dialkyldithiophosphate, phosphite, phosphate, phosphonate, and ammonium phosphate salts, or mixtures thereof. Examples of zinc dithiophosphates include zinc isopropyl methylamyl dithiophosphate, zinc isopropyl isooctyl dithiophosphate, zinc di(cyclohexyl) dithiophosphate, zinc isobutyl 2-ethylhexyl dithiophosphate, zinc isopropyl 2-ethylhexyl dithiophosphate, zinc isobutyl isoamyl dithiophosphate, zinc isopropyl n-butyl dithiophosphate, and combinations thereof. Zinc dialkyldithiophosphate may be present in the lubricating composition in amount to provide 0.01 wt % to 0.1 wt % phosphorus to the lubricating composition, or to provide 0.015 wt % to 0.075 wt % phosphorus, or 0.02 wt % to 0.05 wt % phosphorus, to the lubricating composition. In certain embodiments, the lubricating composition is free or substantially free of a zinc dialkyldithiophosphate. The antiwear agent may be present in the lubricating composition in an amount of 0 wt % to 3 wt %, or 0.1 wt % to 1.5 wt %, or 0.5 wt % to 0.9 wt %, based on the total weight of the lubricating composition.

In certain embodiments, the lubricating composition may further comprise at least one ashless antiwear agent (e.g., present in an amount of 0.01 to 5 wt %, or 0.1 to 2 wt %, based on the total weight of the lubricating composition), such as an ashless antiwear agent represented by the formula:

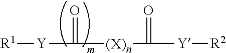

wherein
Y and Y′ are independently —O—, >NH, >NR³, or an imide group formed by taking together both Y and Y′ groups and forming a R¹—N<group between two >C═O groups;
X is independently —Z—O—Z′—, >CH₂, >CHR⁴, >CR⁴R⁵, >C(OH)(CO₂R²), >C(CO₂R²)₂, or >CHOR⁶;
Z and Z′ are independently >CH₂, >CHR⁴, >CR⁴R⁵, >C(OH)(CO₂R²), or >CHOR⁶;
n is 0 to 10, with the proviso that when n=1, X is not >CH₂, and when n=2, both X's are not >CH₂;
m is 0 or 1;
R¹ is independently hydrogen or a hydrocarbyl group, typically containing 1 to 150 carbon atoms, with the proviso that when R¹ is hydrogen, m is 0, and n is more than or equal to 1;
R² is a hydrocarbyl group, typically containing 1 to 150 carbon atoms;
R³, R⁴ and R⁵ are independently hydrocarbyl groups; and
R⁶ is hydrogen or a hydrocarbyl group, typically containing 1 to 150 carbon atoms.

In certain embodiments, the ashless antiwear agent may be a compound obtained/obtainable by a process comprising reacting a glycolic acid, a 2-halo-acetic acid, or a lactic acid, or an alkali or alkaline metal salt thereof, (such as glycolic acid or a 2-halo-acetic acid) with at least one member selected from the group consisting of an amine, an alcohol, and an amino alcohol. For example the compound may be represented by at least one of the following formulae:

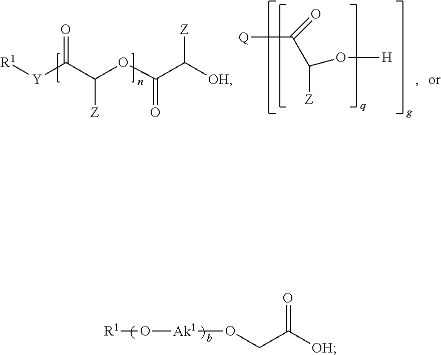

wherein
Y is independently oxygen or >NH or >NR¹;
R¹ is independently a hydrocarbyl group, typically containing 4 to 30, or 6 to 20, or 8 to 18 carbon atoms;
Z is hydrogen or methyl;
Q is the residue of a diol, triol or higher polyol, a diamine, triamine, or higher polyamine, or an aminoalcohol (e.g., Q is a diol, diamine or aminoalcohol)
g is 2 to 6, or 2 to 3, or 2;
g is 1 to 4, or 1 to 3 or 1 to 2;
n is 0 to 10, 0 to 6, 0 to 5, 1 to 4, or 1 to 3; and
Ak¹ is an alkylene group containing 1 to 5, or 2 to 4 or 2 to 3 (such as ethylene) carbon atoms; and
b is 1 to 10, or 2 to 8, or 4 to 6, or 4.

### ILLUSTRATIVE INDUSTRIAL APPLICATIONS

The compositions described herein may be useful as/in liquid fuel compositions, in oils of lubricating viscosity (e.g., lubricating compositions), and/or as lubricating compositions, such as in an internal combustion engine. The internal combustion engine may be a gasoline or diesel engine. Exemplary internal combustion engines include, but are not limited to, spark ignition and compression ignition engines; 2-stroke or 4-stroke cycles; liquid fuel supplied via direct injection, indirect injection, port injection and carburetor; homogeneous charge compression ignition engines; common rail and unit injector systems; light (e.g. passenger car) and heavy duty (e.g. commercial truck) engines; and engines fueled with hydrocarbon and non-hydrocarbon fuels and mixtures thereof. The engines may be part of integrated emissions systems incorporating such elements as; EGR systems; aftertreatment including three-way catalyst, oxidation catalyst, NOₓ absorbers and catalysts, catalyzed and non-catalyzed particulate traps optionally employing fuel-borne catalyst; variable valve timing; and injection timing and rate shaping.

In certain embodiments, the subject matter disclosed herein may be used with diesel engines having direct fuel injection systems wherein the fuel is injected directly into the engine's combustion chamber. The ignition pressures may be greater than 1000 bar and, in one embodiment, the ignition pressure may be greater than 1350 bar. Accordingly, the direct fuel injection system may be a high-pressure direct fuel injection system having ignition pressures greater than 1350 bar. Exemplary types of high-pressure direct fuel injection systems include, but are not limited to, unit direct injection (or “pump and nozzle”) systems, and common rail systems. In unit direct injection systems, the high-pressure fuel pump, fuel metering system, and fuel injector are combined into one apparatus. Common rail systems have a series of injectors connected to the same pressure accumulator, or rail. The rail in turn, is connected to a high-pressure fuel pump. The unit direct injection or common rail systems may further comprise an optional turbocharged or supercharged direct injection system.

In certain embodiments, the subject matter disclosed herein may be useful for providing at least equivalent, if not improved detergency (deposit reduction and/or prevention) performance in both the traditional and modern diesel engine compared to conventional additives. In addition, the technology may provide similar or improved water shedding (or demulsifying) performance compared to conventional compounds in both the traditional and modern diesel engine.

In yet another embodiment, a lubricating composition comprising the reaction product comprising a quaternary ammonium salt may be useful for lubricating an internal combustion engine (e.g., for crankcase lubrication).

Embodiments of the present technology may provide at least one of antiwear performance, friction modification (particularly for enhancing fuel economy), detergent performance (particularly deposit control or varnish control), or dispersancy (particularly soot control, sludge control, or corrosion control).

### EXAMPLES

The subject matter disclosed herein may be better understood with reference to the following examples, which are set forth merely to further illustrate the subject matter disclosed herein. The illustrative examples should not be construed as limiting the subject matter in any manner.

### Preparatory Material A

Polyisobutylene succinic anhydride (1113.6 g: 1.74 mol), which was prepared by reacting 550 Mₙ high vinylidene polyisobutylene and maleic anhydride using the radical functionalization process described herein (which produces the radically-functionalized PIBSA product), was heated to 70° C. and charged to a 2 L round bottomed flange flask fitted with stirrer, condenser with Dean-Stark trap, nitrogen inlet, subline addition pipe, and thermocouple/temperature control system. N,N-dimethylaminopropylamine (179.3 g: 1.74 mol) was then charged via subline addition pipe over 90 minutes, maintaining batch temperature below 120° C. The batch was then heated to 150° C. and held on temperature for 3 hours, collecting approximately 30 mL of water. The resulting product, comprising a non-quaternized radically functionalized succinimide detergent, was cooled and collected.

### Preparatory Material B

Polyisobutylene succinic anhydride (1899.7 g: 1.72 mol), which was prepared by reacting 1000 Mₙ high vinylidene polyisobutylene and maleic anhydride using the radical functionalization process described herein (which produces the radically-functionalized PIBSA product), was heated to 70° C. and charged to a 3 L round bottomed flange flask fitted with stirrer, condenser with Dean-Stark trap, nitrogen inlet, subline addition pipe, and thermocouple/temperature control system. N,N-dimethylaminopropylamine (176.0 g: 1.72 mol) was then charged via subline addition pipe over 60 minutes, maintaining batch temperature below 120° C. The batch was then heated to 150° C. and held on temperature for 3 hours, collecting approximately 30 mL of water. The resulting product, comprising a non-quaternized radically functionalized succinimide detergent, was cooled and collected.

### Preparatory Material C

Polyisobutylene succinic anhydride (700.3 g: 1.10 mol), which was prepared by reacting 550 Mₙ high vinylidene polyisobutylene and maleic anhydride using a conventional “Ene” reaction, was heated to 70° C. and charged to a 2 L round bottomed flange flask fitted with stirrer, condenser with Dean-Stark trap, nitrogen inlet, subline addition pipe, and thermocouple/temperature control system. N,N-dimethylaminopropylamine (112.3 g: 1.10 mol) was then charged via subline addition pipe over 90 minutes, maintaining batch temperature below 120° C. The batch was then heated to 150° C. and held on temperature for 3 hours, collecting approximately 30 mL of water. The resulting product, comprising a non-quaternized succinimide detergent, was cooled and collected.

### Preparatory Material D

Polyisobutylene succinic anhydride (2000 g: 1.81 mol), which was prepared by reacting 1000 Mₙ high vinylidene polyisobutylene and maleic anhydride using the conventional “Ene” reaction, was heated to 70° C. and charged to a 3 litre round bottomed flange flask fitted with stirrer, condenser with Dean-Stark trap, nitrogen inlet, subline addition pipe, and thermocouple/temperature control system. N—N-dimethylaminopropylamine (185.2 g: 1.81 mol) was then charged via subline addition pipe over 60 minutes, maintaining batch temperature below 120° C. The batch was then heated to 150° C. and held on temperature for 3 hours, collecting approx. 30 mL of water. The resulting product, comprising a non-quaternized succinimide detergent, was cooled and collected.

### Example 1

Preparatory Material A (848.8 g: 1.13 mol) was charged to a 2 L round bottomed flange flask fitted with stirrer, condenser with dry-ice/acetone trap, nitrogen inlet, and thermocouple/temperature control system. 2-ethylhexanol (255.6 g: 1.96 mol), acetic acid (68.1 g: 1 0.13 mol), and water (26.6 g: 1.46 mol) were added to the flask and the mixture was stirred with an overhead stirrer and heated to 70° C. Propylene oxide (139.7 g: 2.41 mol) was then added sub surface via a PTFE headed peristaltic pump over a period of ~2 hours. The batch was held at 70° C. for 4 hours. Heating was then switched off and the batch blown via subsurface dip-leg for 60 minutes, to remove residual propylene oxide. The resulting product, comprising a radically functionalized based quaternized succinimide detergent with an acetate counter ion, was cooled and collected.

### Example 2

Preparatory Material A (321.2 g: 0.44 mol) was charged to a 1 L round bottomed flange flask fitted with stirrer, condenser with dry-ice/acetone trap, nitrogen inlet, and thermocouple/temperature control system. 2-ethylhexanol (116.4 g: 0.90 mol), tall oil fatty acid (TOFA, 113.8 g: 0.44 mol), and water (8.7 g: 0.48 mol) were added to the flask and the mixture was stirred with an overhead stirrer and heated to 70° C. Propylene oxide (54.1 g: 0.93 mol) was then added sub surface via a PTFE headed peristaltic pump over a period of ~60 minutes. The batch was held at 70° C. for 4 hours. Heating was then switched off and the batch blown via subsurface dip-leg for 60 minutes, to remove residual propylene oxide. The resulting product, comprising a radically functionalized based quaternized succinimide detergent with TOFA counter ion, was cooled and collected.

### Example 3

Preparatory Material B (399.5 g: 0.34 mol) was charged to a 1 L round bottomed flange flask fitted with stirrer, condenser with dry-ice/acetone trap, nitrogen inlet, and thermocouple/temperature control system. 2-ethylhexanol (153.6 g: 1.18 mol), acetic acid (21.0 g: 0.35 mol), and water (4.0 g: 0.22 mol) were added to the flask and the mixture was stirred with an overhead stirrer and heated to 70° C. Propylene oxide (36.4 g: 0.63 mol) was then added sub surface via a syringe pump over a period of ~2 hours. The batch was held at 70° C. for 4 hours. Heating was then switched off and the batch blown via subsurface dip-leg for 60 minutes, to remove residual propylene oxide. The resulting product, comprising a radically functionalized based quaternized succinimide detergent with an acetate counter ion, was cooled and collected.

### Example 4

Preparatory Material C (952.6 g: 1.32 mol) was charged to a 2 L round bottomed flange flask fitted with stirrer, condenser with dry-ice/acetone trap, nitrogen inlet, and thermocouple/temperature control system. 2-ethylhexanol (305.9 g: 2.35 mol), acetic acid (83.8 g: 1.39 mol), and water (28.7 g: 1.58 mol) were added to the flask and the mixture was stirred with an overhead stirrer and heated to 70° C. Propylene oxide (154 g: 2.65 mol) was then added sub surface via a PTFE headed peristaltic pump over a period of ~2 hours. The batch was held at 70° C. for 90 minutes. Heating was then switched off and the batch blown via subsurface dip-leg for 60 minutes, to remove residual propylene oxide. The resulting product, comprising a quaternized succinimide detergent with an acetate counter ion, was cooled and collected.

### Example 5

Preparatory Material C (337.1 g: 0.45 mol) was charged to a 1 L round bottomed flange flask fitted with stirrer, condenser with Dry-Ice/acetone trap, nitrogen inlet, and thermocouple/temperature control system. 2-ethylhexanol (122.8 g: mol), TOFA (112 g: 0.45 mol), and water (10.1 g: 0.56 mol) were added to the flask and the mixture was stirred with an overhead stirrer and heated to 70° C. Propylene oxide (56.8 g: 0.98 mol) was then added sub surface via a PTFE headed peristaltic pump over a period of ~60 minutes. The batch was held at 70° C. for 4 hours. Heating was then switched off and the batch blown via subsurface dip-leg for 60 minutes, to remove residual propylene oxide. The resulting product, comprising a quaternized succinimide detergent with TOFA counter ion, was cooled and collected.

### Example 6

Preparatory Material D (726.2 g: 0.70 mol) was charged to a 2 L round bottomed flange flask fitted with stirrer, condenser with dry-ice/acetone trap, nitrogen inlet, and thermocouple/temperature control system. 2-ethylhexanol (306.3 g: 2.35 mol), acetic acid (42.6 g: 0.71 mol), and water (12.8 g: 0.71 mol) were added to the flask and the mixture was stirred with an overhead stirrer and heated to 70° C. Propylene oxide (89.7 g: 1.54 mol) was then added sub surface via a PTFE headed peristaltic pump over a period of ~2 hours. The batch was held at 70° C. for 90 minutes. Heating was then switched off and the batch blown via subsurface dip-leg for 60 minutes, to remove residual propylene oxide. The resulting product, comprising a quaternized succinimide detergent with an acetate counter ion, was cooled and collected.

### Solubility Testing

The Examples may be tested for solubility in fuel using the following test method: 0.1 wt % of the Example compound may be added to reference fuel RF79-07 (referred to as RF79) and slightly mixed. This solution may then be further diluted with reference fuel RF-79 to prepare 15 ppm (by weight) solutions. If the sample shows precipitation, the Example compound which was tested may not be suitable for use in a fuel.

### Demulsification Testing

The Demulsification Test (ASTM D1094-07, “Standard Test Method for Water Reaction of Aviation Fuels”) is performed to measure an Example's ability to demulsify fuel and water mixtures. The Example compound is added to room temperature fuel at various ppm by weight based on the total weight of the fuel.

The fuel (80 mL, RF79) including the Example compound is then added to a clean, 100 mL-graduated cylinder. A phosphate buffer solution with a pH of 7.0 (20 mL) is then added to the graduated cylinder and the cylinder is stoppered. The cylinder is shaken for 2 minutes at 2 to 3 strokes per second and placed on a flat surface. The volume of the aqueous layer, or water recovery, is then measured at several time points, such as at 3, 5, 7, 10, 15, and 30-minute intervals. Results are reported as an average of 2 measurements.

Example 1 through 6 were tested according to the Demulsification Test at the amounts (ppm by weight) shown in Table 1, with the amount of water recovered at each time interval reported in mL, as shown in Table 1.

TABLE 1

Time Interval (min)

| | 3 | 5 | 7 | 10 | 15 | 30 |
|---|---|---|---|---|---|---|
| Ex. 4, 60 ppm | 4 | 4 | 4 | 4 | 4.5 | 4.5 |
| Ex. 1, 60 ppm | 5 | 6 | 6 | 6.5 | 7 | 8 |
| Ex. 4, 30 ppm | 2.5 | 2.5 | 3 | 3.5 | 3.5 | 4 |
| Ex. 1, 30 ppm | 2 | 3 | 3 | 3.5 | 4.5 | 6.5 |
| Ex. 6, 15 ppm | 2 | 2.5 | 5 | 4 | 4 | 4 |
| Ex. 3, 15 ppm | 7 | 7.5 | 7.5 | 7.5 | 8 | 8.5 |
| Ex. 6, 5 ppm | 8.5 | 8.5 | 9 | 9.5 | 10 | 10 |
| Ex. 3, 5 ppm | 8 | 9.5 | 10 | 10 | 10 | 15 |
| Ex. 5, 15 ppm | 1 | 2 | 2 | 2 | 2 | 3 |
| Ex. 2, 15 ppm | 3 | 4.5 | 5 | 6 | 8.5 | 10 |

The Examples based on radically-functionalized PIBSA products generally demulsified the water/fuel mixtures faster, as compared with the Examples based on conventionally-made PIBSAs.

### Package Testing

A series of 4 packages were prepared as shown in Table 2, with the amounts shown as parts of each additive added to the package.

TABLE 2

| Component | Package 1 | Package 2 | Package 3 | Package 4 |
|---|---|---|---|---|
| Example 4 | 27.26 | — | — | — |
| Example 1 | — | 27.26 | — | — |
| Example 5 | — | — | 27.26 | — |
| Example 2 | — | — | — | 27.26 |
| Demulsifier | 1.60 | 1.60 | 1.60 | 1.60 |
| Solvent | 71.14 | 71.14 | 71.14 | 71.14 |

Each of Packages 1 through 4 were added to RF79 at the treat rates (reported in ppm by weight) to deliver quaternary ammonium salt levels (“Quat Level”, reported in ppm by weight) in each fuel as shown in Table 3.

TABLE 3

| | Package | Treat Rate | Quat Level |
|---|---|---|---|
| Example A | 1 | 48 | 13 |
| Example B | 2 | 48 | 13 |
| Example C | 1 | 117 | 32 |
| Example D | 2 | 117 | 32 |
| Example E | 3 | 117 | 32 |
| Example F | 4 | 117 | 32 |
| Example G | 1 | 250 | 68 |
| Example H | 2 | 250 | 68 |
| Example I | 3 | 250 | 68 |
| Example J | 4 | 250 | 68 |
| Example K | 1 | 338 | 92 |
| Example L | 2 | 338 | 92 |

The fuels of Examples A through L were tested according to the Demulsification Test, with the amount of water recovered at each time interval reported in mL, as shown in Table 4.

TABLE 4

| | Time Interval (min) | | | | | |
|---|---|---|---|---|---|---|
| | 3 | 5 | 7 | 10 | 15 | 30 |
| Example A | 0 | 0 | 0 | 1 | 7 | 20 |
| Example B | 0 | 3 | 9 | 14 | 20 | 20 |
| Example C | 2 | 2 | 3 | 3 | 5.5 | 16 |
| Example D | 0.5 | 7 | 10 | 19 | 20 | 20 |
| Example E | 0 | 0 | 0 | 1 | 3.5 | 15 |
| Example F | 0 | 2.5 | 7.5 | 18.5 | 20 | 20 |
| Example G | 9 | 12 | 12 | 13 | 13 | 17 |
| Example H | 8 | 13 | 17 | 20 | 20 | 20 |
| Example I | 7 | 8 | 9 | 10 | 11 | 12 |
| Example J | 12 | 15 | 18 | 20 | 20 | 20 |
| Example K | 0 | 0 | 0 | 0.5 | 0.5 | 0.5 |
| Example L | 3 | 9.5 | 14 | 20 | 20 | 20 |

### Preparatory Material E

550 Mₙ Polyisobutylene (1100 g: 2.00 mol) is charged to a 2 litre Buchi Polyclave vessel followed by citraconic anhydride (235.4 g; 2.10 mol). The vessel is then sealed and pressure tested to ensure no leaks. After pressure testing the vessel is purged 4 times with nitrogen, to remove oxygen from the system. The vessel is then pressurised to 1 barg before initiating agitation (350 rpm). The Huber oil circulation unit is then set to 222° C., internal, and heating is initiated. Once the batch temp reaches 216° C. it is deemed on temperature and held for 6 hours (pressure attain was ~60 psi). After 6 hours on temp the batch is cooled and sampled for IR to confirm reaction had gone to completion. The material made is methyl 550 Mₙ polyisobutylene succinic anhydride.

### Preparatory Material F

Material E (1000 g; 1.70 mol) is added to a 2 litre flange vessel with over-head stirrer, sub-surface addition tube, water condenser with Dean Stark apparatus, thermocouple and nitrogen inlet. The material is then heated to 90° C., stirring initiated at 200 rpm and nitrogen blanket also initiated. Once the batch reaches 90° C. dimethylaminopropylamine (173.7 g; 1.70 mol) is charged via the sub-surface addition tube, maintaining the batch temp below 120° C. The batch is then heated to 155° C., and water (30.6 g: 1.7 mol) collected in Dean Stark, and maintained at this temperature for 3 hours. The batch is then cooled back to 60° C. and decanted. The material made is methyl 550Mₙ polyisobutylene succinimide.

### Preparatory Material G

Material F (700 g; 1.04 mol) is added to a 1 litre flange vessel with over-head stirrer, peristaltic pump with sub-surface addition tube, acetone dry ice condenser, thermocouple and nitrogen inlet, followed by 2-ethylhexanol (205 g; 1.58 mol), acetic acid (62.9 g; 1.04 mol) and water (14 g; 0.50 mol). The material is then heated to 70° C., stirring initiated at 200 rpm and nitrogen blanket also initiated. Once the batch reached 70° C. propylene oxide (120.7 g; 2.08 mol) is charged via the peristaltic pump with sub-surface addition tube, maintaining the batch temp between 70-75° C. The batch is maintained at this temperature for 4 hours and then sampled for IR to ensure reaction is complete. The batch is then cooled back to 50° C. and blown with nitrogen sparge to remove excess propylene oxide before being decanted. The material made was methyl 550 Mₙ polyisobutylene succinimide quaternary ammonium salt.

Except in the Examples, or where otherwise explicitly indicated or required by context, all numerical quantities in this description specifying amounts of materials, reaction conditions, molecular weights, number of carbon atoms, and the like, are to be understood as modified by the word “about”. As used herein, the term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value. In other embodiments, the value is within a range of the explicitly-described value which would be understood by those of ordinary skill, based on the disclosures provided herein, to perform substantially similarly to compositions including the literal amounts described herein.

It is to be understood that the upper and lower amount, range, and ratio limits set forth herein may be independently combined, and that any amount within a disclosed range is contemplated to provide a minimum or maximum of a narrower range in alternative embodiments (with the proviso, of course, that the minimum amount of a range must be lower than the maximum amount of the same range). Similarly, the ranges and amounts for each element of the subject matter disclosed herein may be used together with ranges or amounts for any of the other elements.

While certain representative embodiments and details have been shown for the purpose of illustrating the subject matter disclosed herein, it will be apparent to those skilled in this art that various changes and modifications may be made therein without departing from the scope of the subject matter. In this regard, the scope of the invention is to be limited only by the following claims.

1. A process for producing a reaction product, the process comprising: reacting a hydrocarbyl polyisobutylene succinic anhydride with a nitrogen-containing compound to form a quaternizable compound; and reacting the quaternizable compound with a quaternizing agent to form the reaction product; wherein the reaction product comprises a di-hydrocarbyl succinic quaternary ammonium salt.

2. The process of claim 1, wherein the nitrogen-containing compound has: (a) a nitrogen atom capable of reacting with the hydrocarbyl polyisobutylene succinic anhydride to form an imide and/or an amide; and (b) at least one quaternizable amino group.

3. The process of claim 1, wherein the quaternizing agent is suitable for converting the quaternizable amino group to a quaternary nitrogen group.

4. The process of claim 1, wherein the quaternizing agent comprises at least one of a dialkyl sulfate, an alkyl halide, an alkyl alkanoate, an aryl alkanoate, a hydrocarbyl substituted carbonate, or a hydrocarbyl epoxide.

5. The process of claim 1, wherein the quaternizing agent comprises a hydrocarbyl epoxide in combination with an acid.

6. The process of claim 1, wherein the di-hydrocarbyl quaternary ammonium salt comprises at least one compound of the following general formulas I or II: 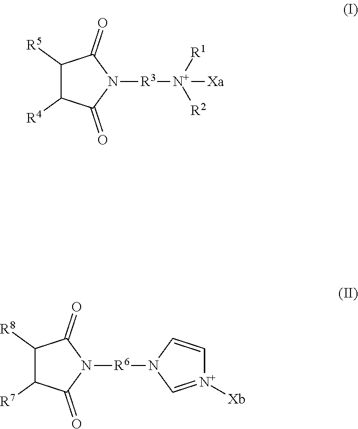
wherein, independently for each molecule of the compound:
each R¹ is a hydrocarbyl group of from 1 to 10 carbon atoms;
each R² is a hydrocarbyl group of from 1 to 10 carbon atoms;
each R³ is a hydrocarbylene group of from 1 to 20 carbon atoms;
each R⁴ is a hydrocarbyl group of from 20 to 180 carbon atoms;
each R⁵ is a hydrocarbyl group of from 1 to 10 carbon atoms;
each R⁶ is a hydrocarbylene group of from 1 to 20 carbon atoms;
each R⁷ is a hydrocarbyl group of from 20 to 180 carbon atoms;
each R⁸ is a hydrocarbyl group of from 1 to 10 carbon atoms;
each Xa is a group derived from the quaternizing agent; and
each Xb is a group derived from the quaternizing agent.

7. The process of claim 1, wherein the hydrocarbyl polyisobutylene succinic anhydride comprises at least one compound of the following general formula III: 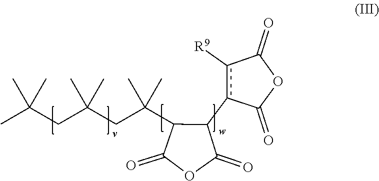
wherein, independently for each molecule:
R⁹ is a hydrocarbyl group of from 1 to 10 carbon atoms or is not present; wherein when R⁹ is not present, the dashed double bond represents a double bond, and when R⁹ is present, the dashed double bond represents a single bond;
v is an integer from 1 to 52; and
w is 0, 1 or 2.

8. The process of claim 7, wherein R⁹ is present and the dashed double bond represents a single bond.

9. The process of claim 8, wherein R⁹ is a methyl group, an ethyl group, or an isobutylene group.

10. The process of claim 8, wherein R⁹ is an isobutylene group.

11. A composition comprising the reaction product of the process of claim 1.

12. A fuel composition comprising a fuel and the composition of claim 11.

13. A lubricating composition comprising an oil of lubricating viscosity and the composition of claim 11.

14. A method of improving water shedding performance of a fuel composition, the method comprising incorporating the composition of claim 11 into the fuel composition.

15. A method of reducing and/or preventing injector deposits in an engine, the method comprising supplying the fuel composition of claim 12 to a fuel injector of the engine and operating the engine.

16. A method of lubricating the crankcase of an engine, the method comprising supplying the lubricating composition of claim 13 to the engine and operating the engine.

17. (canceled)

18. (canceled)

19. A composition comprising a di-hydrocarbyl quaternary ammonium salt according to at least one of the following general formulas I or II: 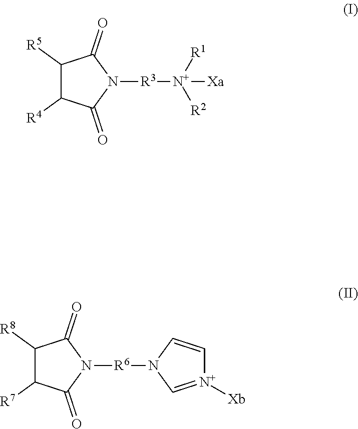
wherein, independently for each molecule of the compound:
each R¹ is a hydrocarbyl group of from 1 to 10 carbon atoms;
each R² is a hydrocarbyl group of from 1 to 10 carbon atoms;
each R³ is a hydrocarbylene group of from 1 to 20 carbon atoms;
each R⁴ is a hydrocarbyl group of from 20 to 180 carbon atoms;
each R⁵ is a hydrocarbylene group of from 1 to 10 carbon atoms;
each R⁶ is a hydrocarbylene group of from 1 to 20 carbon atoms;
each R⁷ is a hydrocarbyl group of from 20 to 180 carbon atoms;
each R⁸ is a hydrocarbylene group of from 1 to 10 carbon atoms;
each Xa is a group derived from the quaternizing agent; and
each Xb is a group derived from the quaternizing agent.

20. A fuel composition comprising a fuel and the composition of claim 19.

21. A lubricating composition comprising an oil of lubricating viscosity and the composition of claim 19.

22. A method of improving water shedding performance of a fuel composition, the method comprising incorporating the composition of claim 19 into the fuel composition.

23. A method of reducing and/or preventing injector deposits in an engine, the method comprising supplying the fuel composition of claim 20 to a fuel injector of the engine and operating the engine.

24. A method of lubricating the crankcase of an engine, the method comprising supplying the lubricating composition of claim 21 to the engine and operating the engine.

25. (canceled)

26. (canceled)
